(19) United States
(12) Patent Application Publication
(10) Pub. No.: US 2016/0354456 A1
(43) Pub. Date: Dec. 8, 2016

(54) METHODS FOR IDENTIFYING TUMOR-SPECIFIC POLYPEPTIDES

(71) Applicant: UNIVERSITY OF CONNECTICUT, Farmington, CT (US)

(72) Inventor: David Han, Farmington, CT (US)

(21) Appl. No.: 15/241,797

(22) Filed: Aug. 19, 2016

Related U.S. Application Data
(63) Continuation of application No. 14/478,907, filed on Sep. 5, 2014.
Application No. 14/478,907 is a division of application No. 13/744,953, filed on Jan. 18, 2013, now abandoned.
(60) Provisional application No. 61/588,105, filed on Jan. 18, 2012.

(51) Int. Cl. A61K 39/00; G01N 33/68

(52) U.S. Cl. CPC A61K 39/0011; G01N 33/6848

(57) ABSTRACT

The present invention provides methods for identifying tumor-specific polypeptides, polypeptides so identified, and methods for their use

### CROSS-REFERENCE

This application is a continuation of U.S. application Ser. No. 14/478907 filed Sep. 5, 2014, which is a divisional of U.S. application Ser. No. 13/744953 filed Jan. 18. 2013, which claims priority to U.S. Provisional Patent Application Ser. No. 61/588105 filed Jan. 18, 2012, each incorporated by reference herewith in their entirety.

### BACKGROUND

The last two decades of research and clinical practice have convincingly demonstrated that the immune system plays a critical surveillance role in detecting tumor-specific antigens and in eliminating cancer cells. Although this research clearly indicates the importance of the immune system in preventing and/or eradicating cancer, the molecular basis for cancer immunity is not comprehensively understood. Furthermore, even with the exponential growth of adaptive immunity strategies seen in recent years, clinical trials of cancer immunotherapy continue to show disappointingly low rates of objective response, particularly for invasive, vascularized cancer. Most cancer treatments still rely on broad-spectrum genotoxic chemotherapeutic agents with severe side effects. Patient- and cancer-specific targeted therapies are mostly in the early phases and very few if any showed wide-spread clinical success with complete eradication of disease. One critical unresolved issue relating to cancer immunogenicity is determining the total number and molecular identities of high-affinity antigens specific to cancer. A more extensive knowledge of tumor-specific antigens and the signaling pathways they impact may facilitate a deeper understanding of the molecular basis of antitumor immunity, why the immune system fails in cancer patients, and how it can be re-empowered to eliminate cancer cells with exquisite sensitivity and specificity.

Thus, new and effective strategies to detect and manage cancer effectively and comprehensively are critically needed.

### SUMMARY OF THE INVENTION

In a first aspect, the present invention provides methods for identifying tumor-specific polypeptides, comprising:

obtaining a tumor polypeptide set from a tumor sample;

identifying polypeptides present in the tumor sample by comparing the tumor polypeptide set with a reference polypeptide set;

obtaining known mutant polypeptides for each identified tumor polypeptide from a mutant polypeptide set; and

identifying tumor-specific polypeptides by combining the tumor polypeptide set and the known mutant polypeptides and removing wild-type polypeptides.

In one embodiment, the methods may further comprise obtaining mass spectra for one or more of the polypeptides, and identifying the one or more polypeptides by the mass spectra. In another embodiment, the methods may further comprise obtaining a sample mass spectra library of polypeptides from a tumor sample, and generating the tumor polypeptide set by converting the mass spectra library to a set of tumor polypeptide sequences. In a still further embodiment, the methods may further comprise obtaining a gene mutation set from the tumor sample; and generating the tumor polypeptide set by translating the DNA in the gene mutation set to amino acid sequences. In another embodiment, the methods may further comprise identifying tumor-specific polypeptides by identifying the polypeptides that are present in both the tumor polypeptide set and the known mutant polypeptides.

In various further embodiments, the methods may further comprise obtaining a tumor-specific mass spectra library from the tumor-specific polypeptides; comparing the sample mass spectra library and the tumor-specific mass spectra library; and identifying additional tumor-specific polypeptides by identifying polypeptides present in the sample mass spectra library and the tumor-specific mass spectra library. In this embodiment, the methods may further comprise obtaining the DNA sequences of the tumor-specific polypeptides from a reference database; identifying DNA sequences present in both the gene mutation set and the DNA sequences of the tumor-specific polypeptides; and identifying additional tumor-specific polypeptides by translating the shared sequences to amino acid sequences. In a further embodiment, the methods may comprise identifying tumor-specific polypeptides by identifying polypeptides that are present only in the tumor polypeptide set.

In a second aspect, the present invention provides methods for generating, a tumor polypeptide signature in a patient, comprising identifying polypeptides specific for a patient's tumor sample according to any embodiment or combination of embodiments of the first aspect of the invention.

In a third aspect, the present invention provides methods for selecting a treatment strategy in a patient, comprising:

generating a tumor polypeptide signature according to the second aspect of the invention;

obtaining the tumor polypeptide signature from one or more other patients who have been favorably treated;

comparing the patient's tumor polypeptide signature with the other patients' tumor polypeptide signatures;

determining the similarity of the signatures; and

selecting a treatment strategy that produced a favorable outcome in the other patient if the signatures are similar.

In one embodiment, the methods may further comprise determining the binding affinities for known tumor antigens of the polypeptides in the patient's signature; determining the binding affinities for known tumor antigens of the polypeptides in the signature of one or more other patients; comparing the polypeptides that display high binding affinities for tumor antigens with the polypeptides that display high binding affinities in the other patient; determining the similarity of the polypeptides with high binding affinities; selecting a treatment strategy that produced a favorable outcome in the other patient if the polypeptides with high binding affinities are similar. In a further embodiment, the known tumor antigen is an HLA receptor.

In a fourth aspect, the present invention provides isolated polypeptides comprising or consisting of one or more of the amino acid sequences according to any one of SEQ ID NO:1-23;

these polypeptides can be used, for example, as vaccines or in methods to generate antibodies and induce an immune response. In a fifth aspect, the present invention provides isolated nucleic acids comprising or consisting of a sequence that encodes a polypeptide according to any one of SEQ ID NO: 1-23, in a sixth aspect, the present invention provides compositions comprising or consisting of two or more of the polypeptides of the fourth aspect of the invention (which can be linked, such as when used as a vaccine), or two or more of the nucleic acids according to the fifth aspect of the invention. In a seventh aspect, the present invention provides binding molecules, including but not limited to antibodies, that selectively bind to at least one of the polypeptides identified as SEQ. ID. Nos. 1-23, and pharmaceutical compositions thereof. In one embodiment, the binding molecule can be combined, with/conjugated to a therapeutic agent for use, for example, in targeting therapeutic agents to a tumor. In one embodiment, the binding molecule can be combined with/conjugated to a detectable label for use, for example, in detectably labeling a tumor or diagnosing cancer in a subject. In one embodiment, the binding molecule such as an antibody against a mutated protein present in the blood or other body fluid (including but not limited to serum, urine, saliva, sweat, breast milk, feces, etc.) of cancer patients can be detected by using so called “peptide microarrays”, in which mutant peptides are immobilized on a solid support or in which mutant peptides labeled with fluorescence or radioactive tracers are used to detect the presence of mutant peptide binding antibodies for diagnosis of cancer.

In an eighth aspect, the present invention provides methods for increasing a patient's immune response to tumor cells, comprising administering one or more of the polypeptides identified as SEQ. ID. No. 1-23 to a patient. In one embodiment, the polypeptides can be administered prior to traditional cancer immunotherapy to enhance efficacy of the immunotherapy. In one embodiment, selection of mutant peptides for anti-cancer vaccines and immunotherapy can be accomplished by using mutant peptide microarrays, in which known available mutations identified from cancer genome sequencing projects can be used to select for specific mutant peptides that invoke strong antibody response in a cancer patient.

In a ninth aspect, the present invention provides arrays comprising a polypeptide set, the set consisting of one or more tumor-specific polypeptides identified by the method according to any embodiment or combination of embodiments of the first aspect of the invention.

In a tenth aspect, the present invention provides methods of generating antigen-HLA receptor complexes, comprising;

identifying tumor-specific polypeptides according any embodiment or combination of embodiments of the first aspect of the invention;

selecting tumor-specific polypeptides that bind to one or more HLA receptors;

obtaining recombinant tumor-specific polypeptides; and

conjugating the recombinant tumor-specific polypeptides with one or more HLA receptors.

In one embodiment, the method may further comprise labeling the recombinant tumor specific polypeptides with a detectable label.

In an eleventh aspect, the present invention provides methods for treating cancer comprising

obtaining a sample from a cancer patient;

sorting cells in the patient sample with one or more of the antigen-HLA receptor complexes of arty embodiment or combination of embodiments of the tenth aspect of the invention;

identifying cancer-specific T-cells in the sample,

growing the cancer-specific T-cells in cell culture; and

administering the cancer-specific I cells to the cancer patient.

In a twelfth aspect, the present invention provides methods for generating a DNA vaccine comprising;

identifying tumor-specific polypeptides according to any embodiment or combination of embodiments of the first aspect of the invention;

identifying the antigenic regions of the tumor-specific polypeptides;

obtaining nucleotide sequences that encode for a peptide that targets the antigenic regions of the tumor-specific polypeptides; and

preparing the DNA sequences as a vector.

In a thirteenth aspect, the present invention provides DNA vaccines comprising a nucleotide sequence encoding a peptide that targets the antigenic regions of a tat tic polypeptide or any embodiment or combination of embodiments of the invention.

### DESCRIPTION OF THE FIGURES

FIG. 1. (A) An approach to enrich tumor epithelial cells from the breast cancers is shown. H&E pictures of pre- and postcore images of a breast tumor is shown, (B) A Coomassie stained gel containing protein extracts from twelve breast cancer samples is shown. The lines demarcate 15 gel slices excised for protein identification.

FIG. 2. Steps involved in the process of mutant peptide identification are outlined. Major steps include creation of cancer-specific database from the compiled list of high-confidence proteins; addition of missense mutations; addition of frame-shift mutations from exonic regions: SEQUEST search of mutant database; detection of mutant peptides.

FIG. 3. Predicting mutant gene saturation in pancreatic, colorectal, breast, and glioblastoma cancers. A running total of identified mutant genes (x-axis) is plotted against the corresponding number of unique mutant genes (y-axis) as each patient is added. (A) Two assumptions are explored in approximating the graph of unique mutant genes versus total mutant genes. The circles represent the 24 samples included in the analysis. (B) The estimated number of samples necessary to reach saturation was then determined by dividing total mutant genes by the average number of mutant genes per sample.

FIG. 4. Exemplary depiction of a spectra identifier 108 configured to communicate, via network 106, with mass spectrometer 102 and client devices 104a, 104.

FIG. 5. Exemplary flowchart of user interface module 201 configured to send and/or receive data to and/or from user input devices such as a keyboard, a keypad, a touch screen, a computer mouse, a track ball, a joystick, a camera, a voice recognition module, and/or other similar devices.

### DETAILED DESCRIPTION OF THE INVENTION

All embodiments disclosed herein can be combined unless the context clearly dictates otherwise. Unless defined otherwise, all terms are defined as understood one of ordinary skill in the art.

As used herein. “obtaining” can be any method of acquiring a data set indicated. For example, a tumor polypeptide set can be obtained in several ways as is known in the art. “Obtaining” a data set of polypeptides includes but is not limited to polypeptide extraction, mass spectrometry identification of a sample and conversion to polypeptide sequences, and retrieving the polypeptides from a previously-derived reference database.

As used herein, “reference database” or “reference polypeptide set” is defined as any database that contains information on DNA sequences, amino acid sequences, or both DNA and amino acid sequences in a preferred embodiment, the reference database or reference polypeptide set also has information on mutations in DNA or amino acid sequences. In other embodiments, the reference database or reference polypeptide set contains mass spectra information on amino acid sequences in the database. Non-limiting examples of a reference database include PubMed GenBank, Uniprot FASTA Release 15.9 and UniprotKB XVL Release 15.9. In certain embodiments, the DNA or protein databases are stored on a computing device as described herein.

The “gene mutation set”, as used herein, as defined as a set of genes from a tumor sample which contain mutations. In some embodiments, this set is generated by comparing the polypeptide sequence from a sample with a wild-type sequence. This set can be generated from any source, including but not limited to a reference database, gene array, or from direct sequencing. In certain embodiments, the whole genome of the sample is sequenced, and the full genome is translated to amino acid sequence.

As used herein, “similar” or “similarity” is defined as a patient signature sharing expression of one or more polypeptides with another patient signature. In some embodiments, a patient signature is considered similar to another if one or more tumor-specific polypeptides or genes are shared between the signatures. In other embodiments, 10 or more polypeptides or genes are shared. In other embodiments, 2, 4, 6, 10, 23, 20, 50, 100, 200, 500, 1000, 5000, or 10000 polypeptides or genes are shared.

As used herein, “tumor cell antigen” is defined as any antigen expressed by a tumor cell. In a preferred embodiment, the tumor cell antigen is expressed on the outside of the cell or is secreted.

As used herein, “binding affinity” is defined as the ability of one molecule to bind to another molecule. When defining binding affinities as “high”, “low”, or any other qualitative definition, any set of accepted differential binding properties can be used. For example, the IEDB web site (www.iedb.org) defines <50 mM as high, 50-500 as intermediate, and >500 as low affinity.

“Selectively binds” as used herein refers to a binding reaction that is determinative of the presence of the protein in a heterogeneous population of proteins and other biologics.

“Targeting” as used herein, means directing the entity to which it is attached (e.g., therapeutic agent or marker) to a target cell, for example to a specific type of tumor cell. Alternatively, “targeting” can also mean preferentially activated at a target tissue, for example a tumor.

“Conjugated” as used herein, means joined. The binding molecule can be conjugated to the agent using any known method, including both covalently or noncovalently joining one molecule to another.

The word “label” when used herein refers to a detectable compound or composition which is conjugated directly or indirectly to the binding molecule. The label may be detectable by itself (e.g. radioisotope labels or fluorescent labels) or, in the case of an enzymatic label, may catalyze chemical alteration of a substrate compound or composition which is detectable. Many detectable labels are well known in the art.

The invention discloses an integrated genomics and proteomics approach termed oncoproteomics in which targeted proteomic screens for detection of genome-wide mutations from cancer are implemented. The invention further identifies cancer-specific genomic mutations at the protein level to determine whether mutations identified in exonic DNA can be detected through proteomic analysis.

To search for cancer-specific mutant proteins, the inventors have utilized proteomic datasets from cancer cells and tissues generated from the laboratory. Any type of human tissue can be used as a sample. In a preferred embodiment, the sample is a tumor sample. In one embodiment, tumor polypeptides are extracted from a tissue sample, as is known in the art, and disclosed in the Examples.

In one aspect, the invention discloses a method for identifying tumor-specific polypeptides, comprising obtaining a tumor polypeptide set from a tumor sample, identifying polypeptides present in the tumor sample by comparing the tumor polypeptide set with a reference polypeptide set, obtaining known mutant polypeptides for each identified tumor polypeptide from a mutant polypeptide set, and identifying tumor-specific polypeptides by combining the tumor polypeptide set and the known mutant polypeptides and removing wild-type polypeptides. Identifying tumor-specific polypeptides according to the method can be applied to all types of cancer. In certain embodiments, the tumor sample is derived from human tissues. Non-limiting examples include breast cancers, pancreatic cancers, liver cancer, skin cancers, leukemia, and melanoma. In other embodiments, the sample is from a cancer cell line.

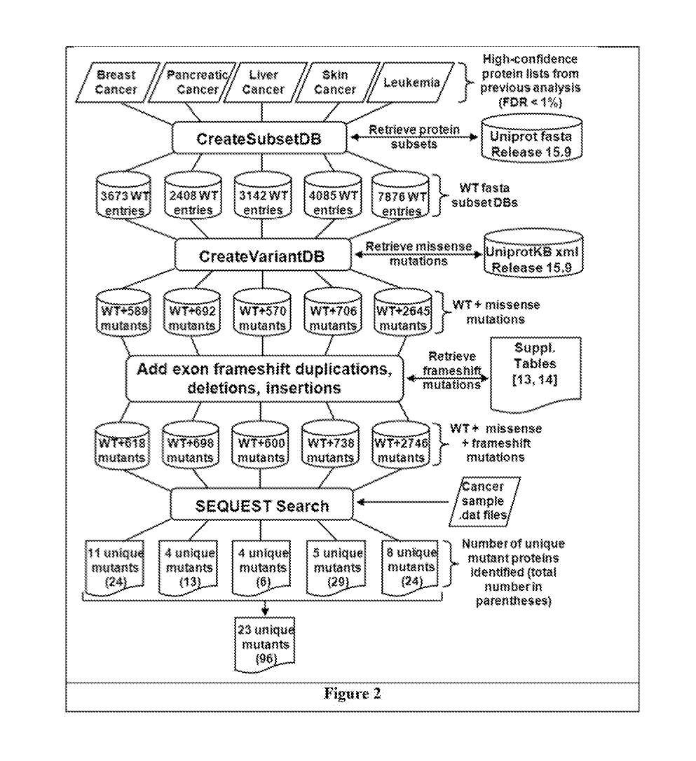

All types of mutant polypeptides are obtained, including missense mutations, frameshift deletions, duplications, and insertions, and any other known mutation. In certain embodiments, the retrieval of mutations are carried out automatically via computer program, such as Java code. At times, additional mutations must be added manually when they are not contained in the reference database of choice, in other embodiments, the genomic variant that matched the reported mutation in the correct position and for the correct number of nucleotides was found, appropriately modified, and translated into its mutant protein counterpart. This mutation can then be added to the other mutant polypeptides. Creation of mutant databases is summarized in the first three steps of FIG. 2.

Once the mutant databases are created, experimentally-generated peptide MS/MS spectra can be re-searched against their respective mutant database to identify other possible cancer-specific mutant peptides (see FIG. 2). In one embodiment, the method further comprises obtaining mass spectra from one or more of the polypeptides, and identifying the one or more polypeptide by the mass spectra. The mass spectra of the invention can be obtained, in any way, including generating the mass spectra from an extracted polypeptide sample, and theoretically using an algorithm. One example of such an algorithm is the SEQUEST algorithm, which allows protein identification from un-interpreted MS/MS spectra. In a preferred embodiment, the tumor cell samples are analyzed using the 1D-GeLC-MS/MS-based protein identification strategy. From one cancer sample, approximately 300,000 MS/MS spectra (sequencing attempts) were generated typically. Each of the spectra has a potential to be identified as a unique peptide.

In another embodiment, the method further comprises obtaining a sample mass spectra library of polypeptides from a tumor sample and generating the tumor polypeptide set by convening the mass spectra library to polypeptide sequences. In this way, the amino acid sequence of the polypeptide can be identified from the mass spectra. In this embodiment, the mass spectra is generated directly from the tumor sample. The generated mass spectra can then be convened to polypeptide sequence. This facilitates identification of the full-length protein affiliated with the extracted polypeptide.

In another embodiment, the method further comprises obtaining a tumor-specific mass spectra library from the tumor-specific polypeptides, and comparing the sample mass spectra library and the tumor-specific mass spectra library, and identifying additional tumor-specific polypeptides by identifying polypeptides present in the sample mass spectra library and the tumor-specific mass spectra library. Mass spectra will not always be readily generated from a protein extract from a tumor sample, because some of the polypeptides are in low quantity or produce poor signal. In this embodiment, a cumulative mutant dataset is generated. This new cumulative mutant dataset can be used to re-analyze the tumor mass spectra and identify the polypeptides that could not be identified in the first pass analysis. In other embodiments, the method further comprises comprising identifying tumor-specific polypeptides by identifying, polypeptides that are present only in the tumor polypeptide set. The mass spectra generated from the tumor sample extraction analysis can be stored and used for future studies.

Tumor-specific polypeptides can also be generated using a genomic approach. DNA sequencing has become more common and is readily available to patients for sequencing of individual patient genomes. The DNA sequence of a patient is becoming a more useful tool for diagnostics. In one embodiment, the method for identifying tumor-specific polypeptides further comprises obtaining a gene mutation set from the tumor sample and generating the tumor polypeptide set by translating the DNA in the gene mutation set to amino acid sequences. In certain embodiments, the DNA sequence can be compared to a reference DNA sequence, and differences identified as the source of potential tumor-specific polypeptides. In certain embodiments, genomic data can be translated theoretically and compared to known amino acid mutations. The amino acid sequences of the sample can be compared to a wild type reference database to determine which polypeptides are mutated when compared to the wild type amino acid sequences.

In another embodiment, the method further comprises identifying tumor-specific polypeptides by identifying the polypeptides that are present in both the tumor polypeptide set and the known mutant polypeptides. In this embodiment, the method seeks to capture the polypeptides that are specific to the particular tumor sample. In certain embodiments, the specific polypeptides are also specific to the patient.

In another embodiment, the method further comprises obtaining the DNA sequences of the tumor-specific polypeptides from a reference database, identifying DNA sequences present in both the gene mutation set and the DNA sequences of the tumor-specific polypeptides, and identifying additional tumor-specific polypeptides by translating the shared sequences to amino acids sequences. In this embodiment, the DNA sequences of the tumor-specific polypeptides are obtained using a reference database. These DNA sequences can be compared to the sequences in the gene mutation set, which will generate additional tumor-specific polypeptides which may be useful in any of the applications described in the invention.

The invention discloses a large-scale shotgun proteomic analysis which can efficiently identify patient-specific mutant proteins directly from human tumor tissue samples.

In another aspect, the invention discloses a method for generating, a tumor polypeptide signature in a patient, comprising identifying polypeptides specific for a patient's tumor sample according to the described methods. This tumor polypeptide signature in a patient will contain a set of all of the tumor-specific polypeptides that have been identified for that particular patient's tumor sample. This signature can have many uses, including but not limited to cancer diagnosis, prognosis, predictions on response to therapy, and cancer treatment choices. Correlations between patients and their expression of certain tumor-specific polypeptides provides essential data which will allow predictions on other patients who share this polypeptide expression signature.

The tumor polypeptide signature of a patient can be compared to the signature of other patients, and cancer treatment can be optimized based on these comparisons. In another aspect, the invention discloses a method for selecting a treatment strategy in a patient, comprising generating a tumor polypeptide signature according to the methods of the invention, obtaining the tumor polypeptide signature from one or more other patients, comparing the patient's tumor polypeptide signature with the other patients tumor polypeptide signatures, determining the similarity of the signatures, selecting a treatment strategy that produced a favorable outcome in the other patient if the signatures are similar.

In one embodiment, the method for selecting a treatment strategy in a patient further comprises determining the binding affinities for known tumor antigens of the polypeptides in the patient's signature, determining the binding affinities for known tumor antigens of the polypeptides in the signature of one or more other patients, comparing the polypeptides that display high binding affinities for tumor antigens with the polypeptides that display high binding affinities in the other patient, determining the similarity of the polypeptides with high binding affinities, and selecting a treatment strategy that produced a favorable outcome in the other patient if the polypeptides with high binding affinities are similar. In certain embodiments, the known tumor antigen is an HLA receptor.

Tumor-specific polypeptides are identified according to the method of the invention. In another aspect, the invention discloses a polypeptide comprising or consisting of one or more of the amino acid sequences according to SEQ. ID. Nos. 1-23. The amino acid sequences of one exemplary set of tumor-specific polypeptides are shown in Table 1. In other embodiments, the polypeptide comprises or consists of a mutated amino acid sequence of the following proteins: Fragile X mental retardation syndrome-related protein 1; Spectrin alpha chain, brain; NADH dehydrogenase [ubiquinone] 1 alpha subcomplex subunit 2; Fibronectin; Cyclin-dependent kinase inhibitor 2A, isoforms 1/2/3; GIP-binding protein Rheb; Fatty acid-binding protein, adipocyte; Drebrin; Histone H4; Double-stranded RNA-specific adenosine deaminase; Myosin-Ib; 3-oxoacyl-[acyl-carrier-protein] synthase, mitochondrial; Titin; CAP-Gly domain-containing linker protein; Mitotic checkpoint serine/threonine-protein kinase BUB1 beta; Rho guanine nucleotide exchange factor 1; Serine-protein kinase ATM; Myeloperoxidase; Xanthine dehydrogenase/oxidase; DNA-dependent protein kinase catalytic subunit; and/or Eukaryotic initiation factor 4A-II. Any mutant in the wild-type sequences of these proteins (SEQ. ID. Nos. 24-46; Table 2) can be identified by the methods of the invention. In one embodiment, the polypeptide comprises or consists of one or more of the amino acid sequences according to SEQ. ID. Nos, 2, 5, 9, 11, or 20. In another aspect, the invention discloses a composition, comprising or consisting of two or more polypeptides selected from SEQ. ID. Nos. 1-23. In certain embodiments, the two or more polypeptides are linked. The polypeptides can be linked by any number of ways as is known in the art, including but not limited to via a covalent bond, via electrostatic interactions, via hydrophobic interactions, or a combination thereof. In another embodiment, the polypeptides are linked via as carrier macromolecule or via as cross-linking agent.

In another embodiment, the polypeptide comprises or consists of a breast cancer tumor-specific polypeptide. In certain embodiments, the polypeptide comprises or consists of SEQ. ID, Nos. 4, 6-7, 10, 12-14, 16-17, 20, or 23.

In another embodiment, the polypeptide comprises or consists of a skin cancer tumor-specific polypeptide. In certain embodiments, the polypeptide comprises or consists of SEQ. ID. Nos. 5, 18, or 21.

In another embodiment, the polypeptide comprises or consists of a liver cancer tumor-specific polypeptide. In certain embodiments, the polypeptide comprises or consists of SEQ. ID, Nos. 3, 9, 11, 15, or 19.

In another embodiment, the polypeptide comprises or consists of a leukemia tumor-specific polypeptide. In certain embodiments, the polypeptide comprises or consists of SEQ. ID. Nos. 1-2, 8, or 22.

In another embodiment, the polypeptide comprises or consists of one or more of the tumor-specific polypeptides that bind tumor specific antigens with higher affinity than the wild-type counterpart polypeptides. In one embodiment, the tumor specific antigen is HLA. In another embodiment, the polypeptide is a mutant of IF4A2. In another embodiment, the polypeptide comprises or consists of any of the sequences listed on Table 3.

In another aspect, the invention discloses an isolated nucleic acid comprising or consisting of a sequence that encodes one or more of the polypeptides identified as SEQ. ID. Nos. 1-23.

Molecules that bind to the tumor-specific polypeptides identified by the invention are useful in several applications, including but not limited to imaging, diagnostics, and targeted treatment. Any use of molecules that bind to the tumor-specific polypeptides identified by the invention is contemplated.

In another aspect, the invention discloses a binding molecule, which selectively binds to at least one of the polypeptides identified as SEQ. ID. Nos. 1-23. In certain embodiments, this means that the molecule binds only one tumor-specific polypeptide and shows little or no binding to other polypeptides. In a particular embodiment, the molecule binds only the tumor-specific polypeptide and shows little or no binding to the corresponding wild-type version of the tumor-specific polypeptide.

Tumor-specific polypeptides will be selected for generating monoclonal antibodies for early detection, risk stratification, and for testing therapeutic modalities. In one embodiment, the binding molecule comprises an antibody. In a certain embodiment, the antibody is an isolated monoclonal antibody. In another embodiment, the antibody binds at least one of the polypeptides identified as SEQ. ID. No. 2, 5, 9, 11, or 20. In another embodiment, the isolated antibody is fully human. In a further embodiment, the invention describes a pharmaceutical composition comprising a pharmaceutically acceptable carrier and a therapeutically effective amount of the antibody. In another embodiment, the array comprises or consists of a breast cancer tumor-specific polypeptide. In certain embodiments, the polypeptide comprises or consists of SEQ. ID. Nos. 4, 6-7, 10, 12-14, 16-17, 20, or 23. In another embodiment, the array comprises or consists of a skin cancer tumor-specific polypeptide. In certain embodiments, the polypeptide comprises or consists of SEQ. ID. Nos. 5, 18, or 21. In another embodiment, the array comprises or consists of a liver cancer tumor-specific polypeptide. In certain embodiments, the polypeptide comprises or consists of SEQ. ID. Nos. 3, 9, 11, 15, or 19. In another embodiment, the array comprises or consists of a leukemia tumor-specific polypeptide. In certain embodiments, the polypeptide comprises or consists of SEQ. ID. Nos. 1-2. 8, or 22.

In another aspect, the invention describes a method for creating an antibody, the method comprising administering one or more polypeptides identified as SEQ. ID, No. 1-23 to an animal to induce an immune response. Monoclonal antibodies ma be made using the hybridoma method first described by Kohler et al., Nature, 256:495 (1975), or may be made by recombinant DNA methods (U.S. Pat. No. 4,816,567). The antibodies of the present invention can be made by any known method. Methods for creating an antibody are well known in the art.

In another aspect, the invention describes a vaccine comprising one or more polypeptides identified as SEQ. ID. No 1-23. In one embodiment, the vaccine selectively binds to a tumor antigen with high affinity. In one embodiment, the vaccine comprises one or more polypeptides identified as SEQ. ID. No. 2, 5, 9, 11, or 20. In one embodiment, the one or more polypeptides of the vaccine are linked. Any arrangement of polypeptides can be used according to the invention. A number of studies have shown that long peptides can elicit a more potent in:MIMIC response than a single epitope, even a highly immunogenic epttope. The invention describes a vaccine using a long peptide derived from the linkage of multiple tumor-specific polypeptides exhibiting high-affinity to a range of tumor antigens. In one embodiment, the tumor antigen is FILA receptor.

In another aspect, the invention describes a method for generating a DNA vaccine. Methods for generating DNA vaccines are well known in the art. In one embodiment, the method comprises identifying tumor-specific polypeptides according to the methods of the invention, identifying the antigenic regions of the tumor-specific polypeptides, obtaining nucleotide sequences that encode for a peptide that targets the antigenic regions of the tumor-specific polypeptides, and inserting the DNA sequences into a vector.

In another embodiment, the invention describes a DNA vaccine. In certain embodiments, the DNA vaccine comprises a nucleotide sequence encoding a peptide that targets the antigenic regions of a tumor-specific polypeptide. In certain embodiments, the tumor-specific polypeptide is identified using the methods of the invention. These DNA vaccines can be administered to patients as a treatment for cancer. In another embodiment, the tumor-specific polypeptide binds FILA. In yet another embodiment, the tumor-specific polypeptide binds HLA with high affinity.

In another aspect, the invention describes a method for increasing a patient's immune response to tumor cells, the method comprising administering the polypeptides identified as SEQ. ID. No. 1-23, or other patient-specific mutated polypeptides as determined by genomic or proteomic sequencing of a patient's tumor and normal cells or tissue. In one embodiment the method comprises administering any of the tumor-specific polypeptides identified using the described methods to a patient. In one embodiment, patient-specific mutant polypeptides are used to generate a peptide microarray to test cancer patient's sera or other fluid, to identify which mutant peptides invoke strong immune response. In one embodiment, the presence of antibodies against mutant peptides in patient's blood can be identified by the peptide microarrays and peptides that show strong immune response can be used as anti-cancer vaccine reagents. In one embodiment, the polypeptides of the method are administered prior to traditional cancer immunotherapy to enhance efficacy. In one embodiment, the method describes a combined therapy, which administers tumor-specific polypeptides first to boost cancer immunity, followed by treatment using mutant-epitope specific monoclonal antibodies to kill patient specific cancer cells.

In another aspect, the invention describes a composition for targeting therapeutic agents to a tumor, comprising the described tumor-specific polypeptide binding molecule, a therapeutic agent, and wherein the binding molecule is conjugated to the therapeutic agent. This targeting composition has a multitude of uses according to the invention. In certain embodiments, tumor-specific polypeptides are selected that are secreted in the serum. In other embodiments, tumor-specific polypeptides are selected that are expressed on the surface of tumor cells.

In another aspect, the invention describes a method for targeting therapeutic agents to a tumor, comprising administering the targeting composition to a patient with a tumor. In one embodiment, the therapeutic agent is administered in a pharmaceutically acceptable amount to kill cancer cells. Any therapeutic agent can be used. In some embodiments, the therapeutic agent is a cytotoxic agent such as a chemotherapeutic agent, a growth inhibitory agent, a toxin (e.g., an enzymatically active toxin of bacterial, fungal, plant, or animal origin, or fragments thereof), or a radioactive isotope (i.e., a radioconjugate). In one embodiment, the method describes a combined therapy, which administers tumor-specific polypeptides first to boost cancer immunity, followed by treatment using mutant-epitope specific monoclonal antibodies to kill patient specific cancer cells.

In another aspect, the invention describes a composition for detecting tumors, which comprises the described tumor-specific polypeptide binding molecule, a detectable label; and wherein the binding molecule is conjugated to the detectable label. This detecting composition has a multitude of uses according to the invention. Detectable labels are well known in the art, as are methods of attaching them to binding molecules. The label may be detectable by itself (e.g. radioisotope labels or fluorescent labels) or, in the case of an enzymatic label, may catalyze chemical alteration of a substrate compound or composition which is detectable. Many detectable labels are well known in the art.

In another aspect, the invention describes a method of targeting a detectable label to a tumor, comprising administering the detecting composition to a patient. Label detection methods are well known in the art. In certain embodiments, the label is detected using immunohistochemistry or immunofluorescence.

In another embodiment, the invention describes a method for cancer detection, comprising targeting a detectable label to a tumor, and assaying the quantity of detectable label. In another embodiment, the method further comprises determining whether the quantity of label detected is an indicator of cancer.

In another aspect, the invention describes an array comprising a polypeptide set, the set consisting of one or more tumor-specific polypeptides identified by the methods of the invention. In certain embodiments, the array consists of 2-10,000 polypeptides. In other embodiments, the array consists of 2, 4, 6, 10, 23, 20, 50, 100, 200, 500, 1000, 5000, or 10000 polypeptides. In another embodiment, the array comprises or consists of a breast cancer tumor-specific polypeptide. In certain embodiments, the polypeptide comprises or consists of SEQ. ID. Nos. 4, 6-7, 10, 12-14, 16-17, 20, or 23. In another embodiment, the array comprises or consists of a skin cancer tumor-specific polypeptide. In certain embodiments, the polypeptide comprises or consists of SEQ. ID. Nos. 5, 18, or 21. In another embodiment, the array comprises or consists of a liver cancer tumor-specific polypeptide. In certain embodiments, the polypeptide comprises or consists of SEQ. ID. Nos. 3, 9, 11, 15, or 19. In another embodiment, the array comprises or consists of a leukemia tumor-specific polypeptide. In certain embodiments, the polypeptide comprises or consists of SEQ. ID. Nos. 1-2, 8, or 22.

In another embodiment, the invention describes a method of generating antigen-HLA receptor complexes. In one embodiment, the antigen-HLA receptor complex are generated by identifying tumor-specific polypeptides according to the listed methods, selecting tumor-specific polypeptides that bind to one or more HLA receptors, obtaining recombinant tumor-specific polypeptides, and linking the recombinant tumor-specific polypeptides with one or more HLA receptors. These antigen-HLA receptor complexes can be used to identify and sort cancer-specific T cells in a patient. The cancer-specific T-cells can be administered to a cancer patient to enhance T-cell mediated killing of cancer cells. Sorting of cancer-specific T cells, growing these cells in cell culture, and infusion or administration of a sufficient number of these T cells to the patient are well known in the art.

In one specific embodiment, the tumor-specific polypeptides that bind to one or more HLA receptors are selected using T2 stabilization assays. T2 stabilization assays are well known in the art. Briefly, the T2 stabilization assay is based upon the ability of peptides to stabilize the MHC class I complex on the surface of the T2 cell line. The T2 cells are incubated with a specific peptide, the stabilized MHC class I complex is detected using a pan-HLA class I antibody, and analyzed typically using flow cytometry. Binding is assessed in relation to a non-binding negative control peptide.

Recombinant tumor-specific polypeptides can be obtained by any methods as is well known in the art, including expression from a nucleotide sequence associated with the polypeptide. The recombinant tumor-specific polypeptides can also be obtained, for example, by producing the polypeptide synthetically. In certain embodiments, the recombinant tumor-specific polypeptides are labeled with a detectable label. In other embodiments, the antigen-HLA receptor complex is in multimeric form, including but not limited to a tetramer.

### Example Computing Device and Environment.

The steps of the methods as disclosed can in some aspects be performed using a computing device. For example, results of a comparison between one or more input spectra generated by a mass spectrometer or similar device (e.g., PIMS spectra) and one or more stored spectra (e.g., spectra stored as in a database) can be carried out in an automated fashion using a computing device acting as a “spectra identifier.”

Upon completion, content related the results of the comparison can be generated by the spectra identifier. For example, the content can include graphs, images, alphanumeric, and/or video content preferably displayed to a user via a graphical user interface on either the spectra identifier or a client device.

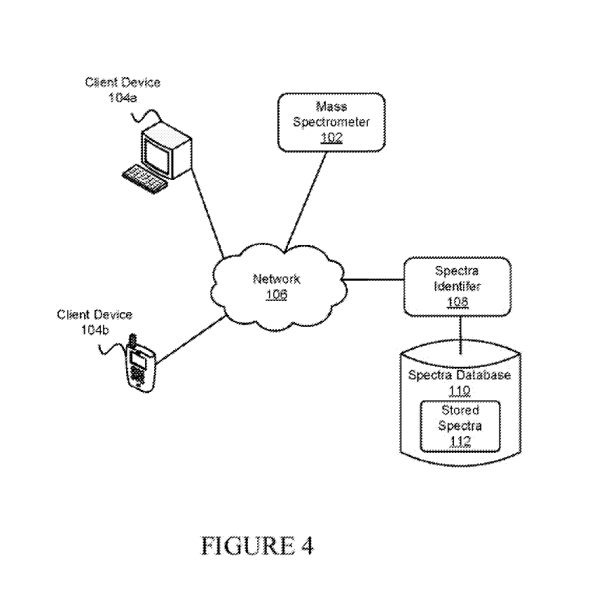

As an example embodiment, FIG. 4 shows spectra identifier 108 configured to communicate, via network 106, with mass spectrometer 102 and client devices 104a, 104b Network 106 may correspond to a LAN, a wide area network (WAN), a corporate intranet, the public Internet, or any other type of network configured to provide a communications path between networked computing devices. The network 106 may also correspond to a combination of one or more LANs, WANs, corporate intranets, and/or the public Internet.

Client devices 104a and 104b (or any additional client devices) may be any sort of computing device, such as an ordinary laptop computer, desktop computer, network terminal, wireless communication device (e.g., a cell phone or smart phone), and so on. In some embodiments, client devices 104a and 104b can be dedicated to research, but it other embodiments, client devices 104a and 104b can be used as general purpose computers that are configured to perform a number of tasks and need not be dedicated to research. In still other embodiments, the functionality of spectra identifier 108 and/or spectra database 110 can be incorporated in a client device, such as client device 104a and/or 104b. In even other embodiments, the functionality of spectra identifier 108 and/or spectra database 110 can be incorporated into mass spectrometer 102.

Spectra identifier 108 can be configured to receive input spectra from mass spectrometer 102 and/or client device(s) 104a and/or 104b via network 106. In some embodiments, spectra identifier can be configured to directly receive input spectra via data input directly to spectra identifier 108, hard-wired connection(s) to mass spectrometer 102 and/or client device(s) 104a and/or 104(b), accessing storage media configured to store input spectra (e.g., spectra database 110, flash media, compact disc, floppy disk, magnetic tape), and/or any other technique to directly provide input spectra to spectra identifier 108.

Spectra identifier 108 can be configured to generate results of spectra identification by comparing one or more input spectra to stored spectra 112. For example, stored spectra 112 can be known precursor on mass spectrometry spectra. As shown in FIG. 4, stored spectra 112 can reside in spectra database 110. When performing spectra identification, spectra identifier 108 can access and/or query spectra database 110 to retrieve part or all of stored spectra 112. In some embodiments, spectra identifier 108 can perform the comparison task directly; while in other embodiments, part or all of the spectra identification task can be performed by spectra database 110, perhaps by executing one or more query language commands upon stored spectra 112.

While FIG. 4 shows spectra identifier 108 and spectra database 110 directly connected, in other embodiments, spectra identifier 108 can include the functionality of spectra database 110, including storing stored spectra 112. In still other embodiments, spectra identifier 108 and spectra database 110 can be connected via network 106.

Upon identifying the input spectra, spectra identifier 108 can be configured to provide content at least related to results of spectra identification, as requested by client devices 104a and/or 104b. The content related to results of spectra identification can include, but is not limited to, web pages, hypertext, scripts, binary data such as compiled software, images, audio, and/or video. The content can include compressed and/or uncompressed content. The content can, be encrypted and/or unencrypted. Other types of content are possible as well.

A computing device (e.g., system) can be configured to perform one or more steps of the disclosed methods. In accordance with an example embodiment, the computing device performs the functions of mass spectrometer 102, client device 104a, 104b, network 106, spectra identifier 108, spectra database 110, and/or stored spectra 112. The computing device may include a user interface module, a network-communication interface module, one or more processors, and data storage, all of which may be linked together via a system bus, network, or other connection mechanism.

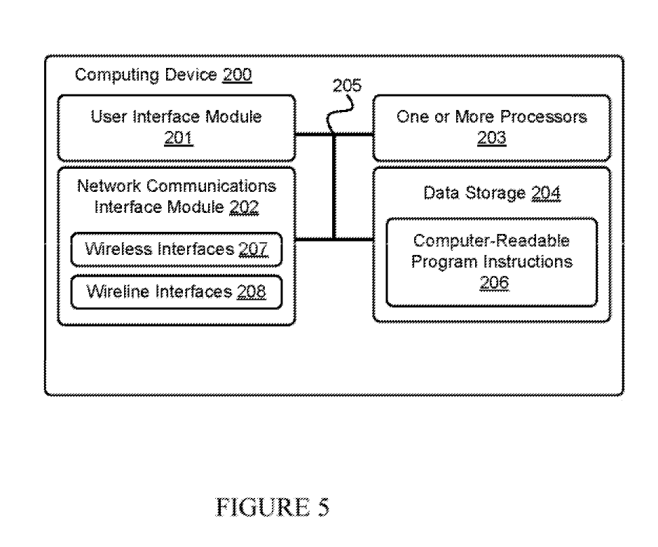

The computing device user can operate an interface to send data to and/or receive data from external user input/output devices. For example, as shown in FIG. 5, a user interface module 201 can be configured to send and/or receive data to and/or from user input devices such as a keyboard, a keypad, a touch screen, a computer mouse, a track ball, a joystick, a camera, a voice recognition module, and/or other similar devices. User interface modules can also be configured to provide output to user display devices, such as one or more cathode ray tubes (CRT), liquid crystal displays (LCD), light emitting diodes (LEDs), displays using digital light processing (DLP) technology, printers, light bulbs, and/or other similar devices, either now known or later developed. User interface modules can also be configured to generate audible output(s), such as a speaker, speaker jack, audio output port, audio output device, earphones, and/or other similar devices. The user interface as well as other computer device components can he connected to a network, as shown in FIG. 5.

Computing processors 203 can include one or more general purpose processors and/or one or more special purpose processors (e.g., digital signal processors, application specific integrated circuits, etc.). Processors can be configured to execute computer-readable program instructions contained in storage and/or other instructions as described herein.

Data storage 204 can include one or more computer-readable storage media that can be read and/or accessed by at least one or more processors 203. The one or more computer-readable storage media can include volatile and/or non-volatile storage components, such as optical, magnetic, organic or other memory or disc storage, which can be integrated in whole or in part with at least one of processors. In some embodiments, data storage can be implemented using a single physical device (e,g., one optical, magnetic, organic or other memory or disc storage unit), while in other embodiments, data storage can be implemented using two or more physical devices. Data storage can include computer-readable program instructions and perhaps additional data. For example, in some embodiments, data storage can store part or all of a spectra database and/or stored spectra, such as spectra database 110 and/or stored spectra 112, respectively. In some embodiments, data storage can additionally include storage required to perform at least part of the herein-described methods and techniques and/or at least part of the functionality of the herein-described devices and networks,

In some embodiments, spectra identifier 108 and spectra database 110 can be a single computing device residing in a single computing center. In other embodiments, spectra identifier 108 and/or spectra database 110 can include multiple computing devices in a single computing center, or even multiple computing devices located in multiple computing centers located in diverse geographic locations. For example, FIG. 4 depicts each of spectra identifier 108 and spectra database 110 residing in different physical locations.

In some embodiments, data and services at spectra identifier 108 and spectra database 110 can be encoded as computer readable information stored in tangible computer readable media (or computer readable storage media) and accessible b client devices 104a and 104b, and/or other computing devices. In some embodiments, data at spectra identifier 108 and/or spectra database 110 can be stored on a single disk drive or other tangible storage media, or can be implemented on multiple disk drives or other tangible storage media located at one or more diverse geographic locations.

### EXAMPLES

### Tumor Sample Preparation.

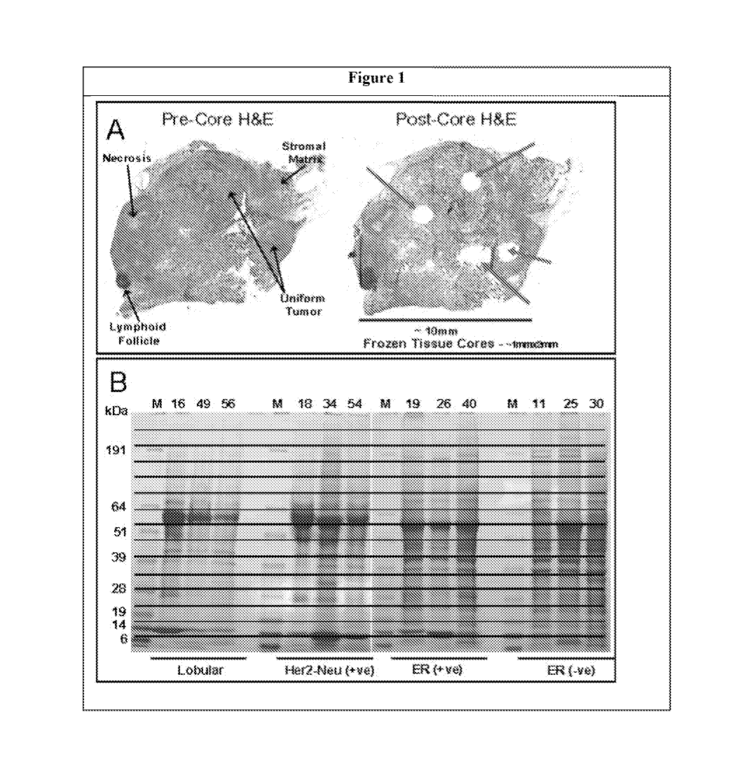

Among many tumor samples, the inventors selected twelve patient's tumor samples based on their estrogen receptor (ER), progesterone receptor (PR), and Her2/Neu expression. Frozen sections were prepared from these twelve samples, stained with Hematoxylin & Eosin, cancer-rich regions located, and cored (FIG. 1A). Proteins from cored tumor samples were then extracted and separated using SDS-PAGE, protein bands from each sample were excised into fifteen gel slices and placed into separate microfuge tubes (FIG. 1B), and tumor-cell derived proteins were in-gel digested into peptides by using trypsin. Once cancer-derived proteins were completely digested, the peptides were extracted from each gel band using a standard peptide extraction buffer (50% Acetonitrile and 5% Formic Acid), The extraction procedure was repeated for four times. Extracted peptides from each gel slice were pooled, loaded onto a C18-reversed phase micro-capillary column using a LC-Packing autosampler, Cancer-derived peptide mixtures from each gel slice were separated using well-established LC-MS/MS procedure as previously described 4. Currently we have the capacity to analyze analyzed one patient's sample per day using available Finnigan LTQ Ion trap mass spectrometers.

### Cancer Data Sets.

Data utilized in this investigation came from previous proteomic analyses of multiple cancer tissues and cell cultures, both published and unpublished, including highly enriched tumor cell samples from two pancreatic cancer patients, one hepatocellular carcinoma patient, twelve breast cancer patients, one melanoma patient and one Merkel cell carcinoma patient. Samples from one lymphocytic leukemia cell line, one melanocyte cell line and five melanoma cell lines were also included in the study. Data sets were all convened to a common format before combining lists to obtain unions. The conversion was done by matching previously identified peptides to entries in the UniProt Knowledgebase Release 15.9 (13 Oct. 2009) fasta-format database. UPSP entries were positioned above UPTR entries in the database, and the first match was retrieved as the converted id. The converted protein ids formed the basis of the mutant databases. The following is a brief description of samples representing each cancer type:

Pancreatic cancer: Highly enriched turner cells and adjacent normal cells from two cancer patients (44T/N and 69T/N), subfractionated and analyzed by LC-MS/MS as previously reported, made up the pancreatic data utilized in this study. A total of 2408 unique proteins were identified in these combined samples.

Liver cancer: Highly enriched tumor cells and adjacent normal cells from one hepatocellular carcinoma patient (55T/N) provided the hepatocellular data utilized in this study. The proteomic methods used to analyze the hepatocellular sample are the same as those described for the pancreatic cancer samples. The hepatocellular data has not yet been published, but the manuscript is currently in submission. The sample comprises 3142 unique proteins,

Breast Cancer: Data from LC-MS/MS analyses of 12 breast cancer samples were utilized in this study. Six samples (three ER+ and three ER−) were previously reported, and six samples (three Lobular and three Her2-Neu) are from unpublished work. All samples were prepared and analyzed. The combined samples represent 3243 identified proteins.

Melanoma/Merkel Cell Carcinoma: One melanoma sample and one Merkel cell carcinoma were derived from the analysis of formalin-fixed paraffin-embedded (FFPE) tissue blocks, prepared as previously described. Additionally, unpublished data from one melanocyte and five melanoma cell lines was used in this study. Standard LC-MS/MS techniques were used for data analysis. Altogether 4085 protein identifications were made from these samples.

Leukemia: Leukemia is represented by a sample from the human Jurkat T leukemic cell line. This sample has been exhaustively studied by replicate analyses, fractionation, enrichment and depletion techniques. 7876 unique proteins (Release 15.9, 13 Oct. 2009) have been identified in our lab from this human Jurkat T leukemic cell line.

### Mutant Database Creation.

RAW files from previous proteomic analyses were converted to .dat files and re-searched with SEQUEST against mutant databases to identify cancer-specific somatic mutations. For this purpose, five mutant databases, representing each of the five major cancer types investigated in this study, were created from the Uniprot 20091019 trembl and sprot dbs (UniprotKB Release 15.9, 10-13-2009). Within each cancer type, data sets were converted to UniProt 10-13-2009 accession ids and then combined to obtain a union of all identified proteins. Amino acid sequences for these wild type entries were obtained from a local copy of the Uniprot human fasta database (downloaded from ftp.expasy.org). Known missense mutations associated with these wild type entries were retrieved from the UniprotKB xml database, searching feature type ‘sequence variant’ for keywords cancer, carcinoma, melanoma, glioma and tumor. Missense mutations which were identified in our samples were verified to be somatic, cancer-specific mutations by a search of the supporting literature.

Frameshift deletions, duplications and insertions from published tables for protein-coding regions were then added to the mutant database. For each frameshift mutation, the exact genomic variant which matched the reported mutation in the correct position and for the correct number of nucleotides was found, appropriately modified, and translated into its mutant protein counterpart. Specifically, cDNA isoforms were obtained from web siteexpasy.ch/tools/blast using tblastn, corresponding as sequences were checked against the Uniprot version to find the matching isoform, the DNA sequence was modified in accordance with the frameshift mutation, and the www.expasy.ch/tools/dna.html translate tool was used to obtain a putative protein sequence from the mutated DNA.

Gene Saturation Prediction.

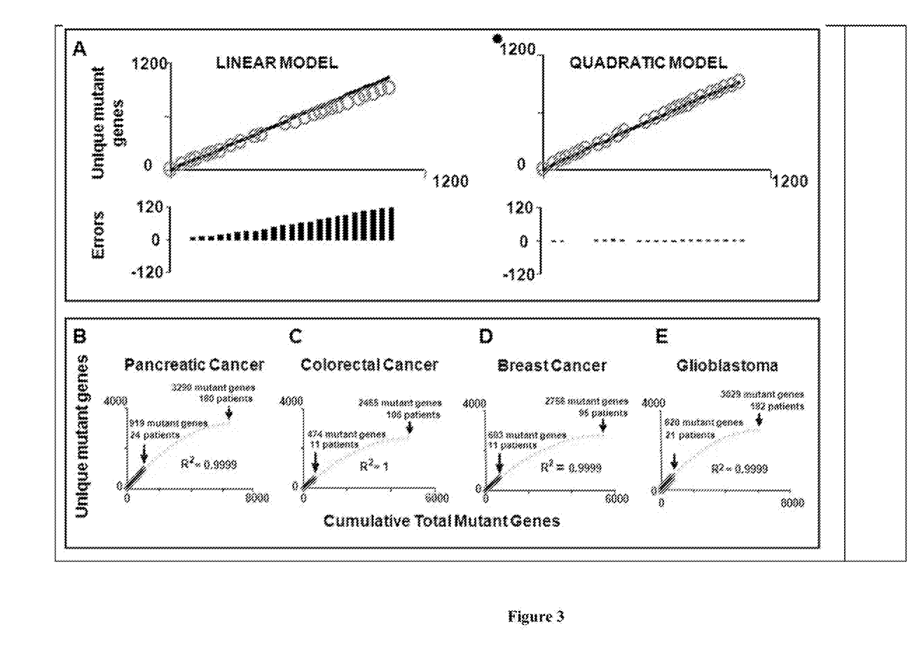

To obtain rough estimates of the number of samples necessary to identify all mutant genes for each cancer type (FIG. 3 the cumulative number of unique mutant genes (y) was plotted against cumulative total mutant genes (x) as each sample was added to the graph. Genes containing missense mutations, plus deletions, duplications and insertions in the coding region were included in the investigation. Three data models were considered a theoretical linear model in which no mutant gene overlap was expected between subsequent samples; a best-fit linear model, in which some chance overlap might occur, but no saturation is expected; and a best-fit quadratic model, in which overlap gradually increases and results in eventual saturation. The quadratic model fit the data best for three of the four cancer types, based on a comparison of the sums of squared errors. Thus, a quadratic equation was fined to each graph in an excel spreadsheet, and saturation was assumed to occur at the point where the slope of the tangent to the graph was equal to 0. X and y values at saturation were computed, and average mutant genes per sample was also calculated. The estimated number of samples necessary to reach saturation was then determined by dividing total mutant genes by the average number of mutant genes per sample.

### Binding Affinity Prediction.

The artificial neural net (ANN) prediction method available at www.iedb.org was used to predict peptide binding affinities to MHC class I molecules. IC50 is the binding affinity measure utilized by the ANN tool. IC50 is the half-maximal inhibitory concentration, measuring the effectiveness of a compound in inhibiting biological or biochemical function. Thus, a lower score corresponds to a higher affinity. The IEDB web site (www.iedb.org) defines <50 mM as high, 50-500 as intermediate, and >500 as low affinity. All available alleles and lengths of the 23 mutant peptides identified in this study (shown in Table 1) were searched against a human database, and predictions with IC50<500 were saved. As an example, a comparison of wild type versus mutant affinities is shown for IF4A2 (Q14240) in Table 3.

TABLE 1
Mutant peptides identified by protomic analysis

| SEQ. | | | | | | Number | | |
|---|---|---|---|---|---|---|---|---|
| ID. | Uniprot | Protein | Mutant | Mutation | Amino | of in- | Mutation | Tissue |
| No. | ID | name | Peptide | (codon) | acid | stances | type | type |
| 1 | P51114 | Fragile X mental | R.EDLMGLTIGTJGSMI | 697G>A | A233T | 1 | missense | leukemia |
| | | retardation syndrome- | QQARK.V | | | | | |
| | | related protein 1 | | | | | | |
| 2 | Q13813 | Spectrin alpha chain, | K.HEAFETNFTYVHK.D | 5752G>A | D1918N | 13 | missense | leukemia |
| | | brain | | | | | | |
| 3 | | NAHDH dehydrogenase | K.ANPNLPHLIR.E | I48G>A | D50N | 1 | missense | liver |
| | | (ubiquinone) 1 alpha | | | | | | |
| | | subcomplex subunit 2 | | | | | | |
| 4 | P02751 | Fibronectin | R.VNVIPVNLPGEHGQR.L | 2818G>A | D940N | 2 | missense | breast |
| 5 | P42771 | Cyclin-dependent | R.RPIQVMM@GSARVAA | c.182_207 | na | 23 | frameshift | skin |
| | | kinase inhibitor 2A, | GLR.R | delAGCG | | | | |
| | | isoform 1/2/3 | | GCTGCTGC | | | | |
| | | | | TCCACGGCG | | | | |
| | | | | CGGAG | | | | |
| 6 | Q15382 | GTP-binding protein | K.ALAKWSNAAFLESS | c.415G>A | E139K | 3 | missense | breast |
| | | Rheb | AK.E | | | | | |
| 7 | P15090 | Fatty acid-binding | K.LVSSENFDDYM# | c.69a>C | E23D | 1 | missense | breast |
| | | protein, adipocyte | KDVGVGFATR.K | | | | | |
| 8 | Q16643 | Drebin | K.SESEVFKAAAILAQR | c.832G>A | E278K | 1 | missense | leukemia |
| | | | PDMPR.E | | | | | |
| 9 | P622805 | Histone H4 | R.GVLKVFLQNVIR.D | c.190G>G | E64Q | 8 | missense | liver |
| 10 | P55268 | Double-stranded RNA- | K.AERMGFTYYTPVTGAS | C2417A>T | E806V | 1 | missense | breast |
| | | specific adenosine | LR.R | | | | | |
| | | deaminase | | | | | | |
| 11 | O43795 | Myosin-Ib | K.ALYPSSVGQFFQGAULK | c.2905G>A | F969K | 7 | missense | liver |
| | | | INK.N | | | | | |
| 12 | Q9NWU1 | 3-oxoacly-[acyl- | R.GSDEGQFNFQNIVSK.S | c.316T>A | F1061 | 2 | missense | breast |
| | | carrier-protein] | | | | | | |
| | | synthase, | | | | | | |
| | | mitochondrial | | | | | | |
| 13 | QSWZ42 | Titin | K.FLFNTFTVLAGEDLK.V | na | L23079F | 6 | missense | breast |
| 14 | P30622 | CAP-Gly domain- | K.LFEERSVLNNQLLE | c.3606G>A | M12131 | 1 | missense | breast |
| | | containing linker | IK.K | | | | | |
| | | protein 1 | | | | | | |
| 15 | O60566 | Mitotic checkpoint | K.EGGALSEATSLEGDEWE | c.44T>C | M15T | 2 | missense | liver |
| | | serine/threonine- | LSK.F | | | | | |
| | | protein kinase BUB1 | | | | | | |
| | | beta | | | | | | |
| 16 | Q92888 | Rho guanine | K.RLM#GVTPWEQFLAQFL | c.493A>G | M165V | 1 | missense | breast |
| | | nucleotide | AQLEAWYGR.D | | | | | |
| | | exchange factor 1 | | | | | | |
| 17 | QSWZ42 | Titin | K.KVDLIQDLPR.V | c.36642G>A | R18881K | 5 | missense | breast |
| 18 | Q13315 | Serine-protein | R>YTVKVQQELKLDFLA | c.7328G>A | R2443Q | 1 | missense | skin |
| | | kinase ATM | LR.A | | | | | |
| 19 | P05164 | Mycloperoxidase | R.LYQEAQKIVGA< | c.1340G>A | R447Q | 2 | missense | liver |
| | | | @VQRTYR.D | | | | | |
| 20 | P47989 | Xanthine | K.MLGVPAMRIVVGVK>R | c.2371C>G | R791G | 8 | missense | breast |
| | | dehydrogenase/ | | | | | | |
| | | oxidase | | | | | | |
| 21 | P78527 | DNA-dependent | K.QLFNSLFSGILK.E | c.8429G>A | S2810N | 1 | missense | skin |
| | | protein kinase | | | | | | |
| | | catatytic subunit | | | | | | |
| 22 | Q13813 | Spectrin alpha | R.RQDLEDSLQAQQYFA | c.711C>G | S904C | 3 | missense | leukemia |
| | | chain, brain | DANEACWM@R.E | | | | | |
| 23 | Q14240 | Eukaryotic | K.MFLLDEADEMLSR.G | c.541G>C | V181L | 3 | missense | breast |
| | | initiation | | | | | | |
| | | factor 4A-H | | | | | | |

TABLE 2
Wile Type Amino Acid Sequences of Whole Proteins Identified

| SEQ ID | Protein (gene) | Amino Acid Sequence |
|---|---|---|
| 24 | Fragile X mental retardation | MAELTVEVRGSNGAFYKGFIKFVHEDSLTVVFENNWQPERQVPFNEVRLPPPPDIKKEIS |
| | syndrome-related protein 1 | EGDEVEVYSRANDQEPCGWWLAKVRMMKGEFYVIEYAACDATYNEIVTFERLRPVNQNKT |
| | (FXR1) | VKKNTFFKCTVDVPEDLREACANENAHKDFKKAVGACRIFYHPETTQLMILSASEATVKR |
| | | VNILSDMHLRSIRTKLMLMSRNEEATKHLECTKQLAAAFHEEFVVREDLMGLAIGTHGSN |
| | | IQQARKVPGVTAIELDEDTGTFRIYGESADAVKKARGFLEFVEDFIQVPRNLVGKVIGKN |
| | | GKVIQEIVDKSGVVRVRIEGDNENKLPREDGMVPFVFVGTKESIGNVQVLLEYHIAYLKE |
| | | VEQLRMERLQIDEQLRQIGSRSYSGRGRGRRGPNYTSGYGTNSELSNPSETESERKDELS |
| | | DWSLAGEDDRDSRHQRDSRRRPGGRGRSVSGGRGRGGPRGGKSSISSVLKDPDSNPYSLL |
| | | DNTESDQTADTDASESHHSTNRRRRSRRRRTDEDAVLMDGMTESDTASVNENGLVTVADY |
| | | ISRAESQSRQRNLPRETLAKNKKEMAKDVIEEHGPSEKAINGPTSASGDDISKLQRTPGE |
| | | EKINTLKEENTQEAAVLNGVS |
| 25 | Spectrin alpha chain, brain | MDPSGVKVLETAEDIQERRQQVLDRYHFFKELSTLRRQKLEDSYRFQFFQRDAEELEKWI |
| | (SPTAN1) | QEKLQIASDENYKDPTNLQGKLQKHQAFEAEVQANSGAIVKLDETGNLMISEGHFASETI |
| | | RTRLMELHRQWELLLEKMREKGIKLLQAQKLVQYLRECEDVMDWINKDEAIVTSEELGQD |
| | | LEHVEVLQKKFEEFQTDMAAHEERVNENVQFAAKLIQEQHPEEELIKTKQDEVNAAWQRL |
| | | KGLALQRQGKLFGAAEVQRFNRDVDETISWIKEKEQLMASDDFGRDLASVQALLRKHEGL |
| | | ERKLAALEDKVKALCAEADRLQQSHRPSATQIQVKREELITNWEQIRTLAAERHARLNDS |
| | | YRLQRFLADFRDLTSWVTEMKALINADELASDVAGAEALLDRHQEHKGEIDAHEDSFKSA |
| | | DESGQALLAAGHYASDEVREKLTVLSEERAALLELWELRRQQYEQCMDLQLFYRDTEQVD |
| | | NWMSKQEAFLLNEDLGDSLDSVEALLKKHEDFEDSLSAQEEKITALDEFATKLIQNNHYA |
| | | MEDVATRRDALLSRRNALHERAMRRRAQLADSFHLQQFFRDSDELKSWVNEKMKTATDEA |
| | | YKDPSNLQGKVQKHQAFEAELSANQSRIDALEKAGQKLIDVNHYAKDEVAARMNEVISLW |
| | | KKLLEATELKGIKLREANQQQQFNRNVEDIELWLYEVEGHLASDDYGKDLTNVQNLQKKH |
| | | ALLEADVAAHQDRIDGITIQARQFQDAGHFDAENIKKKQEALVARYEALKEPMVARKQKL |
| | | ADSLRLQQLFRDVEDEETWIREKEPIAASTNRGKDLIGVQNLLKKHQALQAEIAGHEPRI |
| | | KAVTQKGNAMVEEGHFAAEDVKAKLHELNQKWEALKAKASQRRQDLEDSLQAQQYFADAN |
| | | EAESWMREKEPIVGSTDYGKDEDSAEALLKKHEALMSDLSAYGSSIQALREQAQSCRQQV |
| | | APTDDETGKELVLALYDYQEKSPREVTMKKGDILTLLSNTNKDWWKVEVNDRQGFVPAAY |
| | | VKKLDPAQSASRENLLEEQGSIALRQEQIDNQTRITKEAGSVSLRMKQVEELYHSLLELG |
| | | EKRKGMLEKSCKKFMLFREANELQQWINEKEAALTSEEVGADLEQVEVLQKKFDDFQKDL |
| | | KANESRLKDINKVAEDLESEGLMAEEVQAVQQQEVYGMMPRDETDSKTASPWKSARLMVH |
| | | TVATFNSILELNEWWRSLQQLAEERSQLLGSAHEVQRFHRDADETKEWIEEKNQALNTDN |
| | | YGHDLASVQALQRKHEGFERDLAALGDKVNSLGETAERLIQSHPESAEDLQEKCTELNQA |
| | | WSSLGKRADQRKAKLGDSHDLQRFLSDFRDLMSWINGIRGLVSSDELAKDVTGAEALLER |
| | | HQEHRTEIDARAGTFQAFEQFGQQLLAHGHYASPEIKQKLDILDQERADLEKAWVQRRMM |
| | | LDQCLELQLFHRDCEQAENWMAAREAFLNTEDKGDSLDSVEALIKKHEDFDKAINVQEEK |
| | | IAALQAFADQLIAAGHYAKGDISSRRNEVLDRWRRLKAQMIEKRSKLGESQTLQQFSRDV |
| | | DEIEAWISEKLQTASDESYKDPTNIQSKHQKHQAFEAELHANADRIRGVIDMGNSLIERG |
| | | ACAGSEDAVKARLAALADQWQFLVQKSAEKSQKLKEANKQQNFNTGIKDFDFWLSEVEAL |
| | | LASEDYGKDLASVNNLLKKHQLLEADISAHEDRLKDLNSQADSLMTSSAFDTSQVKDKRD |
| | | TINGRFQKIKSMAASRRAKLNESHRLHQFFRDMDDEESWIKEKKLLVGSEDYGRDLTGVQ |
| | | NLRKKHKRLEAELAAHEPAIQGVLDTFKKLSDDNTIGKEEIQQRLAQFVEHWKELKQLAA |
| | | ARGQRLEESLEYQQFVANVEEEEAWINEKMTLVASEDYGDTLAAIQGLLKKHEAFETDFT |
| | | VHKDRVNDVCTNGQDLIKKNNHHEENISSKMKGLNGKVSDLEKAAAQRKAKLDENSAFLQ |
| | | FNWKADVVESWIGEKENSLKTDDYGRDLSSVQTLLTKQETFDAGLQAFQQEGIANITALK |
| | | DQLLAAKHVQSKAIEARHASLMKRWSQLLANSAARKKKLLEAQSHRFKVEDLFLTFAKKA |
| | | SAFNSWFENAEEDLTDPVRCNSLEEIKALREAHDAFRSSLSSAQADFNQLAELDRQIKSF |
| | | RVASNPYTWFTMEALEETWRNKQKIIKERELELQKEQRRQEENKDLRQEFAQHANAFHQW |
| | | IQETRTYLLDGSCMVEESGTLESQLEATKRKHQEIRAMRSQLKKIEDLGAAMEEALILDN |
| | | KYTEHSTVGLAQQWDQLDQLGMRMQHNLEQQIQARNTTGVTEEALKEFSMMFKHFDKDKS |
| | | GRLNHQEFKSCLRSLGYDLPMVEEGEPDPEFEAILDTVDPNRDGHVSLQEYMAFMISRET |
| | | ENVKSSEEIESAFRALSSEGKPYVTKEELYQNLTREQADYCNSHMKPYVDGKGRELPTAF |
| | | DYVEFTRSLFVN |
| 26 | NADH dehydrogenase | MAAAAASRGVGAKLGLREIRIHLCQRSPGSQGVRDFIEKRYVELKKANPDLPILIRECSD |
| | [ubiquinone] 1 alpha | VQPKLWARYAFGQETNVPLNNFSADQVTRALENVLSGKA |
| | subcomplex subunit 2 | |
| | (NDUFA2) | |
| 27 | Fibronectin (FN1) | MLRGPGPGLLLLAVQCLGTAVPSTFASKSKRQAQQMVQPQSPVAVSQSKPGCYDNGKHYQ |
| | | INQQWERTYLGNALVCTCYGGSRGFNCESKPEAEETCFDKYFGNTYRVGDTYERPDKSMI |
| | | WDCTCIGAGRGRISCTIANRCHEGGQSYKIGDTWRRPHETGGYMLECVCLGNGKGEWTCK |
| | | PIAEKCFDHAAGTSYVVGETWEKPYQGWMMVDCTCLGEGSGRITCTSRNRCNDQDTRTSY |
| | | RIGDTWSKKDNRGNLLQCICTGNGRGEWKCERHTSVQTTSSGSGPFTDVRAAVYQPQPHP |
| | | QPPPYGHCVTDSGVVYSVGMQWLKTQGNKQMLCTCLGNGVSCQETAVTQTYGGNSNGEPC |
| | | VLPFTYNGRTFYSCTTEGRQDGHLWCSTTSNYEQDQKYSFCTDHTVLVQTRGGNSNGALC |
| | | HFPFLYNNHHYTDCTSEGRRDNMKWCGTTQNYDADQKFGFCPMAAHEEICTTNEGVMYRI |
| | | GDQWDKQHDMGHMMRCTCVGNGRGEWTCIAYSQLRDQCIVDDITYNVNDTFHKRHEEGHM |
| | | LNCTCFGQGRGRWKCDPVDQCQDSETGTGYQIGDSWEKYVHGVRYQCYCYGRGIGEWHCQ |
| | | PLQTYPSSSGPVEVFITETPSQPNSHPIQWNAPQPSHISKYILRWRPKNSVGRWKEATIP |
| | | GHLNSYTIKGLKPGVVYEGQLISIQQYGHQEVTRFDFTTTSTSTPVTSNTVTGETTPFSP |
| | | LVATSESVTEITASSFVVSWVSASDTVSGFRVEYELSEEGDEPQYLDLPSTATSVNIPDL |
| | | LPGRKYIVNVYQISEDGEQSLILSTSQTTAPDAPPDTTVDQVDDTSIVVRWSRPQAPITG |
| | | YRIVYSPSVEGSSTELNLPETANSVTLSDLQPGVQYNITIYAVEENQESTPVVIQQETTG |
| | | TPRSDTVPSPRDLQFVEVTDVKVTIMWTPPESAVTGYRVDVIPVNLPGEHGQRLPISRNT |
| | | FAEVTGLSPGVTYYFKVFAVSHGRESKPLTAQQTTKLDAPTNLQFNVETDSTVLVRWTPP |
| | | RAQITGYRLTVGLTRRGQPRQYNVGPSVSKYPLRNLQPASEYTVSLVAIKGNQESPKATG |
| | | VFTTLQPGSSIPPYNTEVTETTIVITWTPAPKIGFKLGVRPSQGGEAPREVTSDSGSIVV |
| | | SGLTPGVEYVYTIQVLRDGQERDAPIVNKVVTPLSPPTNLHLEANPDTGVLTVSWERSTT |
| | | PDITGYRITTTPTNGQQGNSLEEVVHADQSSCTFDNLSPGLEYNVSVYTVKDDKESVPIS |
| | | DTIIPAVPPPTDLRFTNIGPDTMRVTWAPPPSIDLTNFLVRYSPVKNEEDVAELSISPSD |
| | | NAVVLTNLLPGTEYVVSVSSVYEQHESTPLRGRQKTGLDSPTGIDFSDITANSFTVHWIA |
| | | PRATITGYRIRHIIPEHFSGRPEDRVPHSRNSITLTNLTPGTEYVVSIVALNGREESPLL |
| | | IGQQSTVSDVPRDLEVVAATPTSLLISWDAPAVTVRYYRITYGETGGNSPVQEFTVPGSK |
| | | STATISGLKPGVDYTITVYAVTGRGDSPASSKPISINYRTEIDKPSQMQVTDVQDNSISV |
| | | KWLPSSSPVTGYRVTTTPKNGPGPTKTKTAGPDQTEMTIEGLQPTVEYVVSVYAQNPSGE |
| | | SQPLVQTAVTNIDRPKGLAFTDVDVDSIKIAWESPQGQVSRYRVTYSSPEDGIHELFPAP |
| | | DGEEDTAELQGLRPGSEYTVSVVALHDDMESQPLIGTQSTAIPAPTDLKFTQVTPTSLSA |
| | | QWTPPNVQLTGYRVRVTPKEKTGPMKEINLAPDSSSVVVSGLMVATKYEVSVYALKDTLT |
| | | SRPAQGVVTTLENVSPPRRARVTDATETTITISWRTKTETITGFQVDAVPANGQTPIQRT |
| | | IKPDVRSYTITGLQPGTDYKIYLYTLNDNARSSPVVIDASTAIDAPSNLRFLATTPNSLL |
| | | VSWQPPRARITGYIIKYEKPGSPPREVVPRPRPGVTEATITGLEPGTEYTIVYIALKNNQ |
| | | KSEPLIGRKKTDELPQLVTLPHPNLHGPEILDVPSTVQKTPFVTHPGYDTGNGIQLPGTS |
| | | GQQPSVGQQMIFEEHGFRRTTPPTTATPIRHRPRPYPPNVGEEIQIGHIPREDVDYHLYP |
| | | HGPGLNPNASTGQEALSQTTISWAPFQDTSEYIISCHPVGTDEEPLQFRVPGTSTSATLT |
| | | GLTRGATYNVIVEALKDQQRHKVREEVVTVGNSVNEGLNQPTDDSCFDPYTVSHYAVGDE |
| | | WERMSESGFKLLCQCLGFGSGHFRCDSSRWCHDNGVNYKIGELWDRQGENGQMMSCTCLG |
| | | NGKGEFKCDPHEATCYDDGKTYHVGEQWQKEYLGAICSCTCFGGQRGWRCDNCRRPGGEP |
| | | SPEGTTGQSYNQYSQRYHQRTNTNVNCPIECFMPLDVQADREDSRE |
| 28 | Cyclin-dependent kinase | MEPAAGSSMEPSADWLATAAARGRVEEVRALLEAGALPNAPNSYGRRPIQVMMMGSARVA |
| | inhibitor 2A, isoforms 1/2/3 | ELLLLHGAEPNCADPATLTRPHGDAAREGFLDTLVVLHRAGARLDVRDAWGRLPVDLAEE |
| | (CDKN2A) | LGHRDVARYLRAAAGGTRGSNHARIDAAEGPSDIPD |
| 29 | GTP-binding protein Rheb | MPQSKSRKIAILGYRSVGKSSLTIQFVEGQFVDSYDPTIENTFTKLITVNGQEYHLQLVD |
| | (RHEB) | TAGQDEYSIFPQTYSIDINGYILVYSVTSIKSFEVIKVIHGKLLDMVGKVQIPIMLVGNK |
| | | KDLHMERVISYEEGKALAESWNAAFLESSAKENQTAVDVFRRIILEAEKMDGAASQGKSS |
| | | CSVM |
| 30 | Fatty acid-binding protein, | MCDAFVGTWKLVSSENFDDYMKEVGVGFATRKVAGMAKPNMIISVNGDVITIKSESTFKN |
| | adipocyte (FABP4) | TEISFILGQEFDEVTADDRKVKSTITLDGGVLVHVQKWDGKSTTIKRKREDDKLVVECVM |
| | | KGVTSTRVYERA |
| 31 | Drebrin (DBN1) | MAGVSFSGHRLELLAAYEVIREESAADWALYTYEDGSDDLKLAASGEGGLQELSGHFEN |
| | | QKVMYGFCSVKDSQAALPKYVLINWVGEDVPDARKCACASHVAKVAEFFQGVDVIVNASS |
| | | VEDIDAGAIGQRLSNGLARLSSPVLHRLRLREDENAEPVGTTYQKTDAAVEMKRINREQF |
| | | WEQAKKEEELRKEEERKKALDERLRFEQERMEQERQEQEERERRYREREQQIEEHRRKQQ |
| | | TLEAEEAKRRLKEQSIFGDHRDEEEETHMKKSESEVEEAAAIIAQRPDNPREFFKQQERV |
| | | ASASAGSCDVPSPFNHRPGSHLDSHRRMAPTPIPTRSPSDSSTASTPVAEQIERALDEVT |
| | | SSQPPPLPPPPPPAQETQEPSPILDSEETRAAAPQAWAGPMEEPPQAQAPPRGPGSPAED |
| | | LMFMESAEQAVLAAPVEPATADATEIHDAADTIETDTATADTTVANNVPPAATSLIDLWP |
| | | GNGEGASTLQGEPRAPTPPSGTEVTLAEVPLLDEVAPEPLLPAGEGCATLLNFDELPEPP |
| | | ATFCDPEEVEGESLAAPQTPTLPSALEELEQEQEPEPHLLTNGETTQKEGTQASEGYFSQ |
| | | SQEEEFAQSEELCAKAPPPVFYNKPPEIDITCWDADPVEEEEGFEGGD |
| 32 | Histone H4 (HIST1H4) | MSGRGKGGKGLGKGGAKRHRKVLRDNIQGITKPAIRRLARRGGVKRISGLIYEETRGVLK |
| | | VFLENVIRDAVTYTEHAKRKTVTAMDVVYALKRQGRTLYGFGG |
| 33 | Double-stranded RNA- | MNPRQGYSLSGYYTHPFQGYEHRQLRYQQPGPGSSPSSFLLKQIEFLKGQLPEAPVIGQK |
| | specific adenosine deaminase | TPSLPPSLPGLRPRFPVLLASSTRGRQVDIRGVPRGVHLRSQGLQRGFQHPSPRGRSLPQ |
| | (ADAR) | RGVDCLSSHFQELSIYQDQEQRILKFLEELGEGKATTAHKLSGKLGTPKKEINRVLYSLA |
| | | KKGKLQKEAGTPPLWKIAVSTQAWNQHSGVVRPDGHSQGAPNSDPSLEPEDRNSTSVSED |
| | | LLEPFIAVSAQAWNQHSGVVRPDSHSQGSPNSDPGLEPEDSNSTSALEDPLEFLDMAEIK |
| | | EKICDYLFNVSDSSALNLAKNIGLTKARDINAVLIDMERQGDVYRQGTTPPIWHLTDKKR |
| | | ERMQIKRNTNSVPETAPAAIPETKRNAEFLTCNIPTSNASNNMVTTEKVENGQEPVIKLE |
| | | NRQEARPEPARLKPPVHYNGPSKAGYVDFENGQWATDDIPDDLNSIRAAPGEFRAIMEMP |
| | | SFYSHGLPRCSPYKKLTECQLKNPISGLLEYAQFASQTCEFNMIEQSGPPHEPRFKFQVV |
| | | INGREFPPAEAGSKKVAKQDAAMKAMTILLEEAKAKDSGKSEESSHYSTEKESEKTAESQ |
| | | TPTPSATSFFSGKSPVTTLLECMHKLGNSCEFRLLSKEGPAHEPHFQYCVAVGAQTFPSV |
| | | SAPSKKVAKQMAAEEAMKALHGEATNSMASDNQPEGMISESLDNLESMMPNKVRKIGELV |
| | | RYLNTNPVGGLELYARSHGFAAEFKLVDQSGPPHEPKFVYQAKVGGRWFPAVCAHSKKQG |
| | | KQEAADAALRVLIGENEKAERMGFTEVTPVTGASLRRTMLLLSRSPEAQPKTLPLTGSTF |
| | | HDQIAMLSHRCFNTLTNSFQPSLLGRKILAAIIMKKDSEDMGVVVSLGTGNRCVKGDSLS |
| | | LKGETVNDCHAEIISRRGFIRFLYSELMKYNSQTAKDSIFEPAKGGEKLQIKKTVSFHLY |
| | | ISTAPCGDGALFDKSCSDRAMESTESRHYPVFENPKQGKLRTKVENGEGTIPVESSDIVP |
| | | TWDGIRLGERLRTMSCSDKILRWNVLGLQGALLTHFLQPIYLKSVTLGYLFSQGHLTRAI |
| | | CCRVTRDGSAFEDGLRHPFIVNHPKVGRVSIYDSKRQSGKTKETSVNWCLADGYDLEILD |
| | | GTRGTVDGPRNELSRVSKKNIFLLFKKLCSFRYRRDLLRLSYGEAKKAARDYETAKNYFK |
| | | KGLKDMGYGNWISKPQEEKNFYLCPV |
| 34 | Myosin-Ib (MYO1B) | MAKMEVKTSLLDNMIGVGDMVLLEPLNEETFINNLKKRFDHSDIYTYIGSVVISVNPYRS |
| | | LPIYSPEKVEEYRNRNFYELSPHIFALSDEAYRSLRDQDKDQCILITGESGAGKTEASKL |
| | | VMSYVAAVCGKGAEVNQVKEQLLQSNPVLEAFGNAKTVRNDNSSRFGKYMDIEFDFKGDP |
| | | LGGVISNYLLEKSRVVKQPRGERNFHVFYQLLSGASEELLNKLKLERDFSRYNYLSLDSA |
| | | KVNGVDDAANFRTVRNAMQIVGFMDHEAESVLAVVAAVLKLGNIEFKPESRVNGLDESKI |
| | | KDKNELKEICELTGIDQSVLERAFSFRTVEAKQEKVSTTLNVAQAYYARDALAKNLYSRL |
| | | FSWLVNRINESIKAQTKVRKKVMGVLDIYGFEIFEDNSFEQFIINYCNEKLQQIFIELTL |
| | | KEEQEEYIREDIEWTHIDYFNNAIICDLIENNTNGILAMLDEECLRPGTVTDETFLEKLN |
| | | QVCATHQHFESRMSKCSRFLNDTSLPHSCFRIQHYAGKVLYQVEGFVDKNNDLLYRDLSQ |
| | | AMWKASHALIKSLFPEGNPAKINLKRPPTAGSQFKASVATLMKNLQTKNPNYIRCIKPND |
| | | KKAAHIFNEALVCHQIRYLGLLENVRVRRAGYAFRQAYEPCLERYKMLCKQTWPHWKGPA |
| | | RSGVEVLFNELEIPVEEYSFGRSKIFIRNPRTLFKLEDLRKQRLEDLATLIQKIYRGWKC |
| | | RTHFLLMKKSQIVIAAWYRRYAQQKRYQQTKSSALVIQSYIRGWKARKILRELKHQKRCK |
| | | EAVTTIAAYWHGTQARRELRRLKEEARNKHAIAVIWAYWLGSKARRELKRLKEEARRKHA |
| | | VAVIWAYWLGLKVRREYRKFFRANAGKKIYEFTLQRIVQKYFLEMKNKMPSLSPIDKNWP |
| | | SRPYLFLDSTHKELKRIFHLWRCKKYRDQFTDQQKLIYEEKLEASELFKDKKALYPSSVG |
| | | QPFQGAYLEINKNPKYKKLKDAIEEKIIIAEVVNKINRANGKSTSRIFLLTNNNLLLADQ |
| | | KSGQIKSEVPLVDVTKVSMSSQNDGFFAFHLKEGSEAASKGDFLFSSDHLIEMATKLYRT |
| | | TLSQTKQKLNIEISDEFLVQFRQDKVDVKFIQGNQKNGSVPTCKRKNNRLLEVAVP |
| 35 | 3-oxoacyl-[acyl-carrier- | MSNCLQNFLKITSTRLLCSRLQQLRSKRKFFGTVPISRLHRRVVITGIGLVTPLGYGTII |
| | protein] synthase, | LVWDRLIGGESGIVSLVGEEYKSIPCSVAAYVPRGSDEGQFNEQNKVSKSDIKSMSSPTI |
| | mitochondrial (OXSM) | MAIGAAELAMKDSGWHPQSEADQVATGVAIGMGMIPLEVVSETALNFQTKGYNKVSPFFV |
| | | PKILVNMAAGQVSIRYKLKGPNHAVSTACTTGAHAVGDSFRFIAHGDADVMVAGGTDSCI |
| | | SPLSLAGFSRARALSTNSDPKLACRPFHPKRDGFVMGEGAAVLVLEEYEHAVQRRARIYA |
| | | EVLGYGLSGDAGHITAPDPEGEGALRCMAAALKDAGVQPEEISYINAHATSTPLDGAAEN |
| | | KAIKHLFKDHAYALAVSSTKGATGHLLGAAGAVEAAFTTLACYYQKLPPTLNLDCSEPEF |
| | | DLNYVPLKAQEWKTEKRFIGLTNSFGFGGTNATLCIAGL |
| 36 | Titin (TTN) | MTTQAPTFTQPLQSVVVLEGSTATFEAHISGFPVPEVSWFRDGQVISTSLPGVQISFSK |
| | | GRAKLTIPAVTKANSGRYSLKATNGSGQATSTAELLVKAETAPPNFVQRLQSMTVRQGSQ |
| | | VRLQVRVTGIPTPVVKFYRDGAEIQSSLDRQISQEDDLYSLLIAEAYPEDSGTYSVNATN |
| | | SVGRATSTAELLVQGEEEVPAKKTKTIVSTAQISESRQTRIEKKIEAHFDARSIATVEMV |
| | | IDGAAGQQLPHKTPHRIPPKPKSRSPTPPSIAAKAQLARQQSPSPIRHSPSPVRHVRAPT |
| | | PSPVRSVSPAARISTSPIRSVRSPLLMRKTQASTVATGPEVPPPWKQEGYVASSSEAEMR |
| | | ETTLTTSTQIRTEERWEGRYGVQEQVTISGAAGAAASVSASASYAAEAVATGAKEVKQDA |
| | | DKSAAVATVVAAVDMARVREPVISAVEQTAQRTTTTAVHIQPAQEQVRKEAEKTAVTKVV |
| | | VAADKAKEQELKSRTKEVITTKQEQMHVTHEQIRKETEKTFVPKVVISAAKAKEQETRIS |
| | | EEITKKQKQVTQEAIRQETEITAASMVVVATAKSTKLETVPGAQEETTTQQDQMHLSYEK |
| | | IMKETRKTVVPKVIVATPKVKEQDLVSRGREGITTKREQVQITQEKMRKEAEKTALSTIA |
| | | VATAKAKEQETILRTRETMATRQEQIQVTHGKVDVGKKAEAVATVVAAVDQARVREPREP |
| | | GHLEESYAQQTTLEYGYKERISAAKVAEPPQRPASEPHVVPKAVKPRVIQAPSETHIKTT |
| | | DQKGMHISSQIKKTTDLTTERLVHVDKRPRTASPHFTVSKISVPKTEHGYEASIAGSAIA |
| | | TLQKELSATSSAQKITKSVKAPTVKPSETRVRAEPTPLPQFPFADTPDTYKSEAGVEVKK |
| | | EVGVSITGTTVREERFEVLHGREAKVTETARVPAPVEIPVTPPTLVSGLKNVTVIEGESV |
| | | TLECHISGYPSPTVTWYREDYQIESSIDFQITFQSGIARLMIREAFAEDSGRFTCSAVNE |
| | | AGTVSTSCYLAVQVSEEFEKETTAVTEKFTTEEKRFVESRDVVMTDTSLTEEQAGPGEPA |
| | | APYFITKPVVQKLVEGGSVVFGCQVGGNPKPHVYWKKSGVPLTTGYRYKVSYNKQTGECK |
| | | LVISMTFADDAGEYTIVVRNKHGETSASASLLEEADYELLMKSQQEMLYQTQVTAFVQEP |
| | | KVGETAPGFVYSEYEKEYEKEQALIRKKMAKDTVVVTRYVEDQEFHISSFEERLIKEIEY |
| | | RIIKTTLEELLEEDGEEKMAVDISESEAVESGFDLRIKNYRILEGMGVTFHCKMSGYPLP |
| | | KIAWYKDGKRIKHGERYQMDFLQDGRASLRIPVVLPEDEGIYTAFASNIKGNAICSGKLY |
| | | VEPAAPLGAPTYIPTLEPVSRIRSLSPRSVSRSPIRMSPARMSPARMSPARMSPARMSPG |
| | | RRLEETDESQLERLYKPVFVLKPVSFKCLEGQTARFDLKVVGRPMPETFWFHDGQQIVND |
| | | YTHKVVIKEDGTQSLIIVPATPSDSGEWTVVAQNRAGRSSISVILTVEAVEHQVKPMFVE |
| | | KLKNVNIKEGSQLEMKVRATGNPNPDIVWLKNSDIIVPHKYPKIRIEGTKGEAALKIDST |
| | | VSQDSAQYTATAINKAGRDTTRCKVNVEVERAEFPEERKLIIPRGTYRAKEIAAPELEPL |
| | | HLRYGQEQWEEGDLYDKEKQQKPFFKKKLTSLRLKRFGPAHFECRLTPIDGPTMVVEWLH |
| | | DGKPLEAANRLRMINEFGYCDLDYGVAYSRDSGIITCRATNKYGTDHTSATLIVKDEKSL |
| | | VEESQLPEGRKGLQRIEELERMAHEGALTGVTTDQKEKQKPDIVLYPEPVRVLEGETARF |
| | | RCRVTGYPQPKVNWYLNGQLIRKSKRFRVRYDGIHYLDIVDCKSYDTGEVKVTAENPEGV |
| | | IEHKVKLEIQQREDFRSVLRRAPEPRPEFHVHEPGKLQFEVQKVDRPVDTTETKEVVKLK |
| | | RAERITHELVPEESEELRSKFKRRTEEGYYEAITAVELKSRKKDESYEELLRKTKDELLH |
| | | WTKELTEEEKKALAEEGKITITPFKPDKIELSPSMEAPKIFERIQSQTVGQGSDAHFRVR |
| | | VVGKPDPECEWYKNGVKIERSDRIYWYWPEDNVCELVIRDVTAEDSASIMVKAINIAGET |
| | | SSHAFLLVQAKQLITFTQELQDVVAKEKDTMATFECETSEPFVKVKWYKDGMEVHEGDKY |
| | | RMHSDRKVHFLSILTIDTSDAEDYSCVLVEDENVKTTAKLIVEGAVVEFVKELQDIEVPE |
| | | SYSGELECIVSPENIEGKWYHNDVELKSNGKYTITSRRGRQNLTVKDVTKEDQGEYSPVI |
| | | DGKKTTCKLKMKPRPIAILQGLSDQKVCEDGIVQLEVKVSLESVEGVWMKDGQEVQPSDR |
| | | VHIVIDKQSHMLLIEDMTKEDAGNYSFTIPALGSLTSGRVSVYSVDVITPLKDVNVIEGT |
| | | KAVLECKVSVPDVTSVKWYLNDEQIKPDDRVQAIVKGTKQRLVINRTHASDEGPYKLIVG |
| | | RVETNCNLSVEKIKIIRGLRDLTCTETQNVVFEVELSHSGIDVLWNFKDKEIKPSSKYKI |
| | | EAHGKIYKLTVLNMMKDDEGKYTFYAGENITSGKLTVAGGAISKPLTDQTVAESQEAVFE |
| | | CEVANPDSKGEWLRDGKHLPLTNNIRSESDGHKRRLIIAATKLDDIGEYTTKVATSKTSA |
| | | KLKVEAVKIKKTLKNLTVTETQDAVFTVELTHPNVKGVQWIKNGVVLESNEKYAISVKGT |
| | | IYSLRIKNCAIVDESVYGFRLGRLGASARLHVETVKIIKKPKDVTALENATVAFEVSVSH |
| | | DTVPVKWFHKNVEIKPSDKHRLVSERKVHKLMLQNISPSDAGEYTAVVGQLECKAKLFVE |
| | | TLHITKTMKNIEVPETKTASFECEVSHFNVPSMWLKNGVEIEMSEKFKIVVQGKLHQLII |
| | | MNTSTEDSAEYTFVCGNDQVSATLTVTPIMITSMLKDINAEEKDTITFEVTVNYEGISYK |
| | | WLKNGVEIKSTDKCQMRTKKLTHSLNIRNVHFGDAADYTFVAGKATSTATLVYEARHIEF |
| | | RKHIKDIKVLEKKRAMFECEVSEPDITVQWMKDDQELQITDRIKIQKEKYVHRLLIPSTR |
| | | MSDAGKYTVVAGGNVSTAKLFVEGRDVRIRSIKKEVQVIEKQRAVVEFEVNEDDVDAHWY |
| | | KDGIEINFQVQERHKYVVERRIHRMFISETRQSDAGEYTFVAGRNRSSVTLYVNAPEPPQ |
| | | VLQELQPVTVQSGKPARKCAVISGRPQPKISWYKEEQLLSTGFKCKFLHDGQEYTLLLIE |
| | | AFPEDAAVYTCEAKNDYGVATTSASLSVEVPEVVSPDQEMPVYPPAIITPLQDTVTSEGQ |
| | | PARFQCRVSGTDLKVSWYSKDKKIKPSRFFRMTQFEDTYQLEIAEAYPEDEGTYTFVASN |
| | | AVGQVSSTANLSLEAPESILHERIEQEIEMEMKEFSSSFLSAEEEGLHSAELQLSKINET |
| | | LELLSESPVYSTKFDSEKEGTGPIFIKEVSNADISMGDVATLSVTVIGIPKPKIQWFFNG |
| | | VLLTPSADYKFVFDGDDHSLIILFTKLEDEGEYTCMASNDYGKTICSAYLKINSKGEGHK |
| | | DTETESAVAKSLEKLGGPCPPHFLKELKPIRCAQGLPAIFEYTVVGEPAPTVTWFKENKQ |
| | | LCTSVYYTIIHNPNGSGTFIVNDPQREDSGLYICKAENMLGESTCAAELLVLLEDTDMTD |
| | | TPCKAKSTPEAPEDFPQTPLKGPAVEALDSEQEIATFVKDTILKAALITEENQQLSYEHI |
| | | AKANELSSQLPLGAQELQSILEQDKLTPESTREFLCINGSIHFQPLKEPSPNLQLQIVQS |
| | | QKTFSKEGILMPEEPETQAVLSDTEKIFPSAMSIEQINSLTVEPLKTLLAEPEGNYPQSS |
| | | IEPPMHSYLTSVAEEVLSPKEKTVSDTNREQRVTLQKQEAQSALILSQSLAEGHVESLQS |
| | | PDVMISQVNYEPLVPSEHSCTEGGKILIESANPLENAGQDSAVRIEEGKSLRFPLALEEK |
| | | QVLLKEEHSDNVVMPPDQIIESKREPVAIKKVQEVQGRDLLSKESLLSGIPEEQRLNLKI |
| | | QICRALQAAVASEQPGLFSEWLRNIEKVEVEAVNITQEPRHIMCMYLVTSAKSVTEEVTI |
| | | IIEDVDPQMANLKMELRDALCAIIYEEIDILTAEGPRIQQGAKTSLQEEMDSFSGSQKVE |
| | | PITEPEVESKYLISTEEVSYFNVQSRVKYLDATPVTKGVASAVVSDEKQDESLKPSEEKE |
| | | ESSSESGTEEVATVKIQEAEGGLIKEDGPMIHTPLVDTVSEEGDIVHLTTSITNAKEVNW |
| | | YFENKLVPSDEKFKCLQDQNTYTLVIDKVNTEDHQGEYVCEALNDSGKTATSAKLTVVKR |
| | | AAPVIKRKIEPLEVALGHLAKFTCEIQSAPNVRFQWFKAGREIYESDKCSIRSSKYISSL |
| | | EILRTQVVDCGEYTCKASNEYGSVSCTATLTVTEAYPPTFLSRPKSLTTFVGKAAKFICT |
| | | VTGTPVIETIWQKDGAALSPSPNWKISDAENKHILELSNLTIQDRGVYSCKASNKFGADI |
| | | CQAELIIIDKPHFILELEPVQSAINKKVHLECQVDEDRKVTVTWSKDGQKLPPGKDYKIC |
| | | FEDKIATLEIPLAKLKDSGTYVCTASNEAGSSSCSATVTVREPPSFVKKVDPSYLMLPGE |
| | | SARLHCKLKGSPVIQVTWFKNNKELSESNTVRMYFVNSEAILDITDVKVEDSGSYSCEAV |
| | | NDVGSDSCSTEIVIKEPPSFIKTLEPADIVRGTNALLQCEVSGTGPFEISWFKDKKQIRS |
| | | SKKYRLFSQKSLVCLEIFSFNSADVGEYECVVANEVGKCGCMATHLLKEPPTFVKKVDDL |
| | | IALGGQTVTLQAAVRGSEPISVTWMKGQEVIREDGKIKMSFSNGVAVLIIPDVQISFGGK |
| | | YTCLAENEAGSQTSVGELIVKEPAKIIERAELIQVTAGDPATLEYTVAGTPELKPKWYKD |
| | | GRPLVASKKYRISFKNNVAQLKFYSAELHDSGQYTFEISNEVGSSSCETTFTVLDRDIAP |
| | | FFTKPLRNVDSVVNGTCRLDCKIAGSLPMRVSWFKDGKEIAASDRYRIAFVEGTASLEII |
| | | RVDMNDAGNFTCRATNSVGSKDSSGALIVQEPPSFVTKPGSKDVLPGSAVCLDSTFQGST |
| | | PLTIRWFKGNKELVSGGSCYITKEALESSLELYLVKTSDSGTYTCKVSNVAGGVECSANL |
| | | FVKEPATFVEKLEPSQLLKKGDATQLACKVTGTPPIKITWFANDREIKESSKHRMSFVES |
| | | TAVLRLTDVGIEDSGEYMCEAQNEAGSDHCSSIVIVKESPYFTKEFKPIEVLKEYDVMLL |
| | | AEVAGTPPFEITWFKDNTILRSGRKYKTFIQDHLVSLQILKFVAADAGEYQCRVTNEVGS |
| | | SICSARVTLREPPSFIKKIESTSSLRGGTAAFQATLKGSLPTIVTWLKDSDEITEDDNIR |
| | | MTFENNVASLYLSGIEVKHDGKYVCQAKNDAGIQRCSALLSVKEPATITEEAVSIDVTQG |
| | | DPATLQVKFSGTKEITAKWFKDGQELTLGSKYKISVTDTVSILKIISTEKKDSGEYTFEV |
| | | QNDVGRSSCKARINVLDLIIPPSFTKKLKKMDSIKGSFIDLECIVAGSHPISIQWFKDDQ |
| | | EISASEKYKFSFHDNTAFLEISQLEGTDSGTYTCSATNKAGHNQCSGHLTVKEPPYFVEK |
| | | PQSQDVNPNTRVQLKALVGGTAPMTIKWFKDNKELHSGAARSVWKDDTSTSLELFAAKAT |
| | | DSGTYICQLSNDVGTATSKATLFVKEPPQFIKKPSPVLVLRNGQSTTFECQITGTPKIRV |
| | | SWYLDGNEITAIQKHGISFIDGLATFQISGARVENSGTYVCEARNDAGTASCSIELKVKE |
| | | PPTFIRELKPVEVVKYSDVELECEVTGTPPFEVTWLKNNREIRSSKKYTLTDRVSVFNLH |
| | | ITKCDPSDTGEYQCIVSNEGGSCSCSTRVALKEPPSFIKKIENTTTVLKSSATFQSTVAG |
| | | SPPISITWLKDDQILDEDDNVYISFVDSVATLQIRSVDNGHSGRYTCQAKNESGVERCYA |
| | | FLLVQEPAQIVEKAKSVDVTEKDPMTLECVVAGTPELKVKWLKDGKQIVPSRYFSMSFEN |
| | | NVASFRIQSMVKQDSGQYTFKVENDFGSSSCDAYLRVLDQNIPPSFTKKLTKMDKVLGSS |
| | | IHMECKVSGSLPISAQWFKDGKEISTSAKYRLVCHERSVSLEVNNLELEDTANYTCKVSN |
| | | VAGDDACSGILTVKEPPSFLVKPGRQQAIPDSTVEFKAILKGTPPFKIKWFKDDVELVSG |
| | | PKCFIGLEGSTSFLNLYSVDASKTGQYTCHVTNDVGSDSCTTMLLVTEPPKFVKKLEASK |
| | | IVKAGDSSRLECKIAGSPEIRVVWFRNEHELPASDKYRMTFIDSVAVIQMNNLSTEDSGD |
| | | FICEAQNPAGSTSCSTKVIVKEPPVFSSFPPIVETLKNAEYSLECELSGTPPFEVVWYKD |
| | | KRQLRSSKKYKIASKNFHTSIHILNVDTSDIGEYHCKAQNEVGSDTCVCTVKLKEPPRFV |
| | | SKLNSLTVVAGEPAELQASIEGAQPIFVQWLKEKEEVIRESENIRITFVENVATLQFAKA |
| | | EPANAGKYICQIKKDGGMEENMATLMVLEPAVIVEKAGPMTVTVGETCTLECKVAGTPEL |
| | | SVEWYKDGKLLTSSQKHKFSFYNKISSLRILSVERQDAGTYTFQVQNNVGKSSCTAVVDV |
| | | SDRAVPPSFTRRLKNTGGVLGASCILECKVAGSSPISVAWFHEKTKIVSGAKYQTTFSDN |
| | | VCTLQLNSLDSSDMGNYTCVAANVAGSDECRAVLTVQEPPSFVKEPEPLEVLPGKNVTFT |
| | | SVIRGTPPFKVNWFRGARELVKGDRCNIYFEDTVAELELFNIDISQSGEYTCVVSNNAGQ |
| | | ASCTTRLFVKEPAAFLKRLSDHSVEPGKSIILESTYTGTLPISVTWKKDGFNITTSEKCN |
| | | IVTTEKTCILEILNSTKRDAGQYSCEIENEAGRDVCGALVSTLEPPYFVTELEPLEAAVG |
| | | DSVSLQCQVAGTPEITVSWYKGDTKLRPTPEYRTYFTNNVATLVFNKVNINDSGEYTCKA |
| | | ENSIGTASSKTVFRIQERQLPPSFARQLKDIEQTVGLPVTLTCRLNGSAPIQVCWYRDGV |
| | | LLRDDENLQTSFVDNVATLKILQTDLSHSGQYSCSASNPLGTASSSARLTAREPKKSPFF |
| | | DIKPVSIDVIAGESADFECHVTGAQPMRITWSKDNKEIRPGGNYTITCVGNTPHLRILKV |
| | | GKGDSGQYTCQATNDVGKDMCSAQLSVKEPPKFVKKLEASKVAKQGESIQLECKISGSPE |
| | | IKVSWFRNDSELHESWKYNMSFINSVALLTINEASAEDSGDYICEAHNGVGDASCSTALT |
| | | VKAPPVFTQKPSPVGALKGSDVILQCEISGTPPFEVVWVKDRKQVRNSKKFKITSKHFDT |
| | | SLHILNLEASDVGEYHCKATNEVGSDTCSCSVKFKEPPRFVKKLSDTSTLIGDAVELRAI |
| | | VEGFQPISVVWLKDRGEVIRESENTRISFIDNIATLQLGSPEASNSGKYICQIKNDAGMR |
| | | ECSAVLTVLEPARIIEKPEPMTVTTGNPFALECVVTGTPELSAKWFKDGRELSADSKHHI |
| | | TFINKVASLKIPCAEMSDKGLYSFEVKNSVGKSNCTVSVHVSDRIVPPSFIRKLKDVNAI |
| | | LGASVVLECRVSGSAPISCGWFQDGNEIVSGPKCQSSFSENVCTLNLSLLEPSDTGIYTC |
| | | VAANVAGSDECSAVLTVQEPPSFEQTPDSVEVLPGMSLTFTSVIRGTPPFKVKWFKGSRE |
| | | LVPGESCNISLEDFVTELELFEVQPLESGDYSCLVTNDAGSASCTTHLFVKEPATFVKRL |
| | | ADFSVETSGPIVLEATYTGTPPISVSWIKDEYLISQSERCSITMTEKSTILEILESTIED |
| | | YAQYSCLIENEAGQDICEALVSVLEPPYFIEPLEHVEAVIGEPATLQCKVDGTPEIRISW |
| | | YKEHTKLRSAPAYKMQFKNNVASLVINKVDHSDVGEYSCKADNSVGAVASSAVLVIKERK |
| | | LPPFFARKLKDVHETLGFPVAFECRINGSEPLQVSWYKDGVLLKDDANLQTSFVHNVATL |
| | | QILQTDQSHIGQYNCSASNPLGTASSSAKLILSEHEVPPFFDLKPVSVDLALGESGTFKC |
| | | HVTGTAPIKITWAKDNREIRPGGNYKMTLVENTATLTVLKVGKGDAGQYTCYASNIAGKD |
| | | SCSAHLGVQEPPRFIKKLEPSRIVKQDEFTRYECKIGGSPEIKVLWYKDETEIQESSKFR |
| | | MSFVDSVAVLEMHNLSVEDSGDYTCEAHNAAGSASSSTSLKVKEPPIFRKKPHPIETLKG |
| | | ADVHLECELQGTPPFHVSWYKDKRELRSGKKYKIMSENFLTSIHILNVDAADIGEYQCKA |
| | | TNDVGSDTCVGSIALKAPPRFVKKLSDISTVVGKEVQLQTTIEGAEPISVVWFKDKGEIV |
| | | RESDNIWISYSENIATLQFSRVEPANAGKYTCQIKNDAGMQECFATLSVLEPATIVEKPR |
| | | SIKVTTGDTCTLECTVAGTPELSTKWFKDGKELTSDNKYKISFFNKVSGLKIINVAPSDS |
| | | GVYSFEVQNPVGKDSCTASLQVSDRTVPPSFTRKLKETNGLSGSSVVMECKVYGSPPISV |
| | | SWFHEGNEISSGRKYQTTLTDNTCALTVNMLEESDSGDYTCIATNMAGSDECSAPLTVRE |
| | | PPSFVQKPDPMDVLTGTNVTFTSIVKGTPPFSVSWFKGSSELVPGDRCNVSLEDSVAELE |
| | | LFDVDTSQSGEYTCIVSNEAGKASCTTHLYIKAPAKFVKRLNDYSIEKGKPLILEGTFTG |
| | | TPPISVTWKKNGINVTPSQRCNITTTEKSAILEIPSSTVEDAGQYNCYIENASGKDSCSA |
| | | QILILEPPYFVKQLEPVKVSVGDSASLQCQLAGTPEIGVSWYKGDTKLRPTTTYKMHFRN |
| | | NVATLVFNQVDINDSGEYICKAENSVGEVSASTFLTVQEQKLPPSFSRQLRDVQETVGLP |
| | | VVFDCAISGSEPISVSWYKDGKPLKDSPNVQTSFLDNTATLNIFKTDRSLAGQYSCTATN |
| | | PIGSASSSARLILTEGKNPPFFDIRLAPVDAVVGESADFECHVTGTQPIKVSWAKDSREI |
| | | RSGGKYQISYLENSAHLTVLKVDKGDSGQYTCYAVNEVGKDSCTAQLNIKERLIPPSFTK |
| | | RLSETVEETEGNSFKLEGRVAGSQPITVAWYKNNIEIQPTSNCEITFKNNTLVLQVRKAG |
| | | MNDAGLYTCKVSNDAGSALCTSSIVIKEPKKPPVFDQHLTPVTVSEGEYVQLSCHVQGSE |
| | | PIRIQWLKAGREIKPSDRCSFSFASGTAVLELRDVAKADSGDYVCKASNVAGSDTTKSKV |
| | | TIKDKPAVAPATKKAAVDGRLFFVSEPQSIRVVEKTTATFIAKVGGDPIPNVKWTKGKWR |
| | | QLNQGGRVFIHQKGDEAKLEIRDTTKTDSGLYRCVAFNEHGEIESNVNLQVDERKKQEKI |
| | | EGDLRAMLKKTPILKKGAGEEEEIDIMELLKNVDPKEYEKYARMYDITDFRGLLQAFELL |
| | | KQSQEEETHRLEIEEIERSERDEKEFEELVSFIQQRLSQTEPVTLIKDIENQTVLKDNDA |
| | | VFEIDIKINYPEIKLSWYKGTEKLEPSDKFEISIDGDRHTLRVKNCQLKDQGNYRLVCGP |
| | | HIASAKLTVIEPAWERKLQDVTLKEGQTCTMTCQFSVPNVKSEWFRNGRILKPQGRHKTE |
| | | VEHKVHKLTIADVRAEDQGQYTCKYEDLETSAELRIEAEPIQFTKRIQNIVVSEHQSATF |
| | | ECEVSFDDAIVTWYKGPTELTESQKYNFRNDGRCHYMTIHNVTPDDEGVYSVIARLEPRG |
| | | EARSTAELYLTTKEIKLELKPPDIPDSRVPIPTMPIRAVPPEEIPPVVAPPIPLLLPTPE |
| | | EKKPPPKRIEVTKKAVKKDAKKVVAKPKEMTPREEIVKKPPPPTTLIPAKAPEIIDVSSK |
| | | AEEVKIMTITRKKEVQKEKEAVYEKKQAVHKEKRVFIESFEEPYDELEVEPYTEPFEQPY |
| | | YEEPDEDYEEIKVEAKKEVHEEWEEDFEEGQEYYEREEGYDEGEEEWEEAYQEREVIQVQ |
| | | KEVYEESHERKVPAKVPEKKAPPPPKVIKKPVIEKIEKTSRRMEEEKVQVTKVPEVSKKI |
| | | VPQKPSRTPVQEEVIEVKVPAVHTKKMVISEEKMFFASHTEEEVSVTVPEVQKEIVTEEK |
| | | IHVAVSKRVEPPPKVPELPEKPAPEEVAPVPIPKKVEPPAPKVPEVPKKPVPEEKKPVPV |
| | | PKKEPAAPPKVPEVPKKPVPEEKIPVPVAKKKEAPPAKVPEVQKRVVTEEKITIVTQREE |
| | | SPPPAVPEIPKKKVPEERKPVPRKEEEVPPPPKVPALPKKPVPEEKVAVPVPVAKKAPPP |
| | | RAEVSKKTVVEEKRFVAEEKLSFAVPQRVEVTRHEVSAEEEWSYSEEEEGVSISVYREEE |
| | | REEEEEAEVTEYEVMEEPEEYVVEEKLHIISKRVEAEPAEVTERQEKKIVLKPKIPAKIE |
| | | EPPPAKVPEAPKKIVPEKKVPAPVPKKEKVPPPKVPEEPKKPVPEKKVPPKVIKMEEPLP |
| | | AKVTERHMQITQEEKVLVAVTKKEAPPKARVPEEPKRAVPEEKVLKLKPKREEEPPAKVT |
| | | EFRKRVVKEEKVSIEAPKREPQPIKEVTIMEEKERAYTLEEEAVSVQREEEYEEYEEYDY |
| | | KEFEEYEPTEEYDQYEEYEEREYERYEEHEEYITEPEKPIPVKPVPEEPVPTKPKAPPAK |
| | | VLKKAVPEEKVPVPIPKKLKPPPPKVPEEPKKVFEEKIRISITKREKEQVTEPAAKVPMK |
| | | PKRVVAEEKVPVPRKEVAPPVRVPEVPKELEPEEVAFEEEVVTHVEEYLVEEEEEYUIEE |
| | | EEFITEEEVVPVIPVKVPEVPRKPVPEEKKPVPVPKKKEAPPAKVPEVPKKPEEKVPVLI |
| | | PKKEKPPPAKVPEVPKKPVPEEKVPVPVPKKVEAPPAKVPEVPKKPVPEKKVPVPAPKKV |
| | | EAPPAKVPEVPKKLIPEEKKPTPVPKKVEAPPPKVPKKREPVPVPVALPQEEEVLFEEEI |
| | | VPEEEVLPEEEEVLPEEEEVLPEEEEVLPEEEEIPPEEEEVPPEEEYVPEEEEFVPEEEV |
| | | LPEVKPKVPVPAPVPEIKKKVTEKKVVIPKKEEAPPAKVPEVPKKVEEKRIILPKEEEVL |
| | | PVEVTEEPEEEPISEEEIPEEPPSIEEVEEVAPPRVPEVIKKAVPEAPTPVPKKVEAPPA |
| | | KVSKKIPEEKVPVPVQKKEAPPAKVPEVPKKVPEKKVLVPKKEAVPPAKGRTVLEEKVSV |
| | | AFRQEVVVKERLELEVVEAEVEEIPEEEEFHEVEEYFEEGEFHEVEEFIKLEQHRVEEEH |
| | | RVEKVHRVIEVFEAEEVEVFEKPKAPPKGPEISEKIIPPKKPPTKVVPRKEPPAKVPEVP |
| | | KKIVVEEKVRVPEEPRVPPTKVPDVLPPKEVVPEKKVPVPPAKKPEAPPPKVPEAPKEVV |
| | | PEKKVPVPPPKKPEVPPTKVPEVPKAAVPEKKVPEAIPPKPESPPPEVPEAPKEVVPEKK |
| | | VPAAPPKKPEVTPVKVPEAPKEVVPEKKVPVPPPKKPEVPPTKVPEVPKVAVPEKKVPEA |
| | | IPPKPESPPPEVFEEPEEVALEEPPAEVVEEPEPAAPPQVTVPPKKPVPEKKAPAVVAKK |
| | | PELPPVKVPEVPKEVVPEKKVPLVVPKKPEAPPAKVPEVPKEVVPEKKVAVPKKPEVPPA |
| | | KVPEVPKKPVLEEKPAVPVPERAESPPPEVYEEPEEIAPEEEIAPEEEKPVPVAEEEEPE |
| | | VPPPAVPEEPKKIIPEKKVPVIKKPEAPPPKEPEPEKVIEKPKLKPRPPPPPPAPPKEDV |
| | | KEKIFQLKAIPKKICVPEKPQVPEKVELTPLKVPGGEKKVRKLLPERKPEPKEEWLKSVL |
| | | RKRPEEEEPKVEPKKLEKVKKPAVPEPPPPKPVEEVEVPTVTKRERKIPEPTKVPEIKPA |
| | | IPLPAPEPKPKPEAEVKTIKPPPVEPEPTPIAAPVTVPVVGKKAEAKAPKEEAAKPKGPI |
| | | KGVPKKTPSPIEAERRKLRPGSGGEKPPDEAPFTYQLKAVPLKFVKEIKDIILTESEFVG |
| | | SSAIFECLVSPSTAITTWMKDGSNIRESPKHRFIADGKDRKLHIIDVQLSDAGEYTCVLR |
| | | LGNKEKTSTAKLVVEELPVRFVKTLEEEVTVVKGQPLYLSCELNKERDVVWRKDGKIVVE |
| | | KPGRIVPGVIGLMRALTINDADDTDAGTYTVTVENANNLECSSCVKVVEVIRDWLVKPIR |
| | | DQHVKPKGTAIFACDIAKDTPNIKWFKGYDEIPAEPNDKTEILRDGNHLYLKIKNAMPED |
| | | IAEYAVEIEGKRYPAKLTLGEREVELLKPIEDVTIYEKESASFDAEISEADIPGQWKLKG |
| | | ELLRPSPTCEIKAEGGKRFLTLRKVKLDQAGEVLYQALNAITTAILTVKEIELDFAVPLK |
| | | DVTVPERRQARFECVLTREANVIWSKGPDIIKSSDKFDIIADGKKHILVINDSQFDDEGV |
| | | YTAEVEGKKTSARLFVTGIRLKFMSPLEDQTVKEGETATFVCELSHEKMHVVWFKNDAKL |
| | | HTSRTVLISSEGKTHKLEMKEVTLDDISQIKAQVKELSSTAQLKVLEADPYFTVKLHDKT |
| | | AVEKDEITLKCEVSKDVPVKWFKDGEEIVPSPKYSIKADGLRRILKIKKADLKDKGEYVC |
| | | DCGTDKTKANVTVEARLIKVEKPLYGVEVFVGETAHFEIELSEPDVHGQWKLKGQPLTAS |
| | | PDCEIIEDGKKHILILHNCQLGMTGEVSFQAANAKSAANLKVKELPLIFITPLSDVKVFE |
| | | KDEAKFECEVSREPKTFRWLKGTQEITGDDRFELIKDGTKHSMVIKSAAFEDEAKYMFEA |
| | | EDKHTSGKLIIEGIRLKFLTPLKDVTAKEKESAVFTVELSHDNIRVKWFKNDQRLHTTRS |
| | | VSMQDEGKTHSITFK0LSIDDTSQIRVEAMGMSSEAKLTVLEGDPYFTGKLQDYTGVEKD |
| | | EVILQCEISKADAPVKWFKDGKEIKPSKNAVIKADGKKRMLILKKALKSDIGQYTCDCGT |
| | | DKTSGKLDIEDREIKLVRPLHSVEVMETETARFETEISEDDIHANWKLKGEALLQTPDCE |
| | | IKEEGKIHSLVLHNCRLDQTGGVDFQAANVKSSAHLRVKPRVIGLLRPLKDVTVTAGETA |
| | | TFDCELSYEDIPVEWYLKGKKLEPSDKVVPRSEGKVHTLTLRDVKLEDAGEVQLTAKDFK |
| | | THANLFVKEPPVEFTKPLEDQTVEEGATAVLECEVSRENAKVKWFKNGTEILKSKKYEIV |
| | | ADGRVRKLVIHDCTPEDIKTYTCDAKDFKTSCNLNVVPPHVEFLRPLTDLQVREKEMARF |
| | | ECELSRENAKVKWFKDGAEIKKGKKYDIISKGAVRILVINKCLLDDEAEYSCEVRTARTS |
| | | GMLTVLEEEAVFTKNLANIEVSETDTIKLVCEVSKPGAEVIWYKGDEEIIETGRYEILTE |
| | | GRKRILVIQNAHLEDAGNYNCRLPSSRTDGKVKVHELAAEFISKPQNLEILEGEKAEFVC |
| | | SISKESFPVQWKRDDKTLESGDKYDVIADGKKRVLVVKDATLQDMGTYVVMVGAARAAAH |
| | | LTVIEKLRIVVPLKDTRVKEQQHVVFNCEVNTEGAKAKWFRNEEAIFDSSKYIILQKDLV |
| | | YTLRIRDAHLDDQANYNVSLTNHRGENVKSAANLIVEEEDLRIVEPLKDIETMEKKSVTF |
| | | WCKVNRLNVTLKWTKNGEEVPFDNRVSYRVDKYKHMLTIKDCGFPDEGEYIVTAGQDKSV |
| | | AELLIIEAPTEFVEHLEDQTVTEFDDAVFSCQLSREKANVKWYRNGREIKEGKKYKFEKD |
| | | GSIHRLIIKDCRLDDECEYACGVEDRKSRARLFVEEIKVEIIRPPQDILEAPGADVVFLA |
| | | ELNKDKVEVQWLRNNMWVQGDKHQMMSECGKIHRLQICDIKPRDQGEYRFIAKDKEARAK |
| | | LELAAAPKIKTADQDLVVDVGKPLTNWVPYDAYPKAEAEWFKENEPLSTKTIDTTAEQTS |
| | | FRILEAKKGDKGRYKIVLQNKHGKAEGFINLKVIDVPGPVRNLEVTETFDGEVSLAWEEP |
| | | LTDGGSKIIGYVVERRDIKRKTWVLATDRAESCEFTVTGLQKGGVEYLFRVSARNRVGTG |
| | | EPVETDNPVEARSKYDVPGPPLNVTITDVNRFGVSLTWEPPEYDGGAEITNYVIELRDKT |
| | | SIRWDTAMTVRAEDLSATVTDVVEGQEYSFRVRAQNRIGVGKPSAATPFVKVADPIERPS |
| | | PPVNLTSSDQTQSSVQLKWEPPLKDGGSPILGYIIERCEEGKDNWIRCNMKLVPELTYKV |
| | | TGLEKGNKYLYRVSAENKAGVSDPSEILGPLTADDAFVEPTMDLSAFKDGLEVIVPNPIT |
| | | ILVPSTGYPRPTATWCFGDKVLETGDRVKMKTLSAYAELVISPSERSDKGIYTLKLENRV |
| | | KTISGEIDVNVIARPSAPKELKFGDITKDSVHLTWEPPDDDGGSPLTGYVVEKREVSRKT |
| | | WTKVMDFVTDLEFTVPDLVQGKEYLFKVCARNKCGPGEPAYVDEPVNMSTPATVPDPPEN |
| | | VKWRDRTANSIFLTWDPPKNDGGSRIKGYIVERCPRGSDKWVACGEPVAETKMEVTGLEE |
| | | GKWYAYRVKALNRQGASKPSRPTEEIQAVDTQEAPEIFLDVKLLAGLTVKAGTKIELPAT |
| | | VTGKPEPKITWTKADMILKQDKRITIENVPKKSTVTIVDSKRSDTGTYIIEAVNVCGRAT |
| | | AVVEVNVLDKPGPPAAFDITDVTNESCLLTWNPPRDDGGSKITNYVVERRATDSEVWHKL |
| | | SSTVKDTNFKATKLIPNKEYIFRVAAENMYGVGEPVQASPITAKYQFDPPGPPTRLEPSD |
| | | ITKDAVTLTWCEPDDDGGSPITGYWVERLDPDTDKWVRCNKMPVKDTTYRVKGLTNKKKY |
| | | RFRVLAENLAGPGKPSKSTEPILIKDPIDPPWPPGKPTVKDVGKTSVKLNWTKPEHDGGA |
| | | KIESYVIEMLXTGTDEWVRVAEGVPTTQHLLPGLMEGQEYSFRVRAVNKAGESEPSEPSD |
| | | PVLCREKLYPPSPPRWLEVINITKNTADLKWTVPEKDGGSPITNYIVEKRDVRRKGWQTV |
| | | DTTVKDTKCTVTPLTEGSLYVFRVAAENAIGQSDYTEIEDSVLAKDTFTTPGPPYALAVV |
| | | DVTKRHVDLKWEPPKNDGGRPIQRYVIEKKERLGTRWVKAGKTAGPDCNFRVTDVIEGTE |
| | | VQFQVRAENEAGVGHPSEPTEILSIEDPTSPPSPPLDLHVTDAGRKHIAIAWKPPEKNGG |
| | | SPIIGYHVEMCPVGTEKWMRVNSRPIKDLKFKVEEGVVPDKEYVLRVRAVNAIGVSEPSE |
| | | ISENVVAKDPDCKPTIDLETHDIIVIEGEKLSIPVPFRAVPVPTVSWHKDGKEVKASDRL |
| | | TMKNDHISAHLEVPKSVRADAGIYTITLENKLGSATASINVKVIGLPGPCKDIKASDITK |
| | | SSCKLTWEPPEFDGGTPILHYVLERREAGRRTVIPVMSGENKLSWTVKDLIPNGEYFFRV |
| | | KAVNKVGGGEYIELKNPVIAQDPKQPPOPPVDVEVHNPTAEAMTITWKPPLYDGGSKIMG |
| | | YIIEKIAKGEERWKRCNEHLVPILTYTAKGLEEGKEYQFRVRAENAAGISEPSRATPPTK |
| | | AVDPIDAPKVILRTSLEVKRGDEIALDASISGSPYPTITWIKDENVIVPEEIKKRAAPLV |
| | | RRRKGEVQEEEPFVLPLTQRLSIDNSKKGESQLRVRDSLRPDHGLYMIKVENDHGIAKAP |
| | | CTVSVLDTPGPPINFVFEDIRKTSVLCKWEPPLDDGGSEIINYTLEKKDKTKPDSEWIVV |
| | | TSTLRHCKYSVTKLIEGKEYLFRVRAENRFGPGPPCVSKPLVAKDPFGPPDAPDKPIVED |
| | | VTSNSMLVKWNEPKDNGSPILGYWLEKREVNSTHWSRVNKSLLNALKANVDGLLEGLTYV |
| | | FRVCAENAAGPGKFSPPSDPKTAHDPISPPGPPIPRVTDTSSTTIELEWEPPAFNGGGEI |
| | | VGYFVDKQLVGTNEWSRCTEKMIKVRQYTVKEIREGADYKLRVSAVNAAGEGPPGETQPV |
| | | TVAEPQEPPAVELDVSVKGGIQIMAGKTLRIPAVVTGRPVPTKVWTKEEGELDKDRVVID |
| | | NVGTKSELIIKDALRKDHGRYVITATNSCGSKFAAARVEVFDVPGPVLDLKPVVFNRKMC |
| | | LLNWSDPEDDGGSEITGFIIERKDAKMMTWRQPIETERSKCDITGLLEGQEYKFRVIAKN |
| | | KFGCGPPVEIGPILAVDPLGPPTSPERLTYTERTKSTITLDWKEPRSNGGSPIQGYIIEK |
| | | RRHDKPDFERVNKRLCPTTSFLVENLDEHQMYEFRVKAVNEIGESEPSLPLNVVIQDDEV |
| | | PPTIKLRLSVRGDTIKVKAGEPVHIPADVTGLPMPKIEWSKNETVIEKPTDALQITKEEV |
| | | SRSEAKTELSIPKAVREDKGTYTVTASNRLGSVFRNVHVEVYDRPSPPRNLAVTDIKAES |
| | | CYLTWDAPLDNGGSEITHYVIDKRDASRKKAEWEEVTNTAVEKRYGIWKLIPNGQYEFRV |
| | | RAVNKYGISOECKSDKVVIQDPYRLPGPPGKPKVLARTKGSMLVSWTPPLDNGGSPITGY |
| | | WLEKREEGSPYWSRVSRAPITKVGLKGVEFNVPRLLEGVKYQFRAMAINAAGIGPPSEPS |
| | | DPEVAGDPIFPPGPPSCPEVKDKTKSSISLGWKPPAKDGGSPIKGYIVEMQEEGTTDWKR |
| | | VNEPDKLITTCECVVPNLKELRKYRFRVKAVNEAGESEPSDTTGEIPATDIQEEPEVFID |
| | | IGAQDCLVCKAGSQIRIPAVIKGRPTPKSSWEFDGKAKKAMKDGVHDIPEDAQLETAENS |
| | | SVIIIPECKRSHTGKYSITAKNKAGQKTANCRVKVMDVPGPPKDLKVSDITRGSCRLSWK |
| | | MPDDDGGDRIKGYVIEKRTIDGKAWTKVNPDCGSTTFVVPDLLSEQQYFFRVRAENRFGI |
| | | GPPVETIQRTTARDPIYPPDPPIKLKIGLITKNTVHLSWKPPKNDGGSPVTHYIVECLAW |
| | | DPTGTKKEAWRQCNKRDVEELQFTVEDLVEGGEYEFRVKAVNAAGVSKPSATVGPCDCQR |
| | | PDMPPSIDLKEFMEVEEGTNVNIVAKIKGVPFPTLTWFKAPPKKPDNKEPVLYDTHVNKL |
| | | VVDDTCTLVIPQSRRSDTGLYTITAVNNLGTASKEMRLNVLGRPGPPVGPIKFESVSADQ |
| | | MTLSWFPPKDDGGSKITNYVIEKREANRKTNWHVSSEPKECTYTIPKLLEGHEYVFRIMA |
| | | QNKYGIGEPLDSEPETARNLFSVPGAPDKPTVSSVTRNSMTVNWEEPEYDGGSPXTGYWL |
| | | EMKDTTSKRWKRVNRDPIKAMTLGVSYKVTGLIEGSDYQFRVYAINAAGVGPASLPSDPA |
| | | TARDPIAPPGPPFPKVTDWTKSSADLEWSPPLKDGGSKVTGYIVEYKEEGKEEWEKGKDK |
| | | EVRGTKLVVTGLKEGAFYKFRVSAVNIAGIGEPGEVTDVIEMKDRLVSPDLQLDASVRDR |
| | | IVWAGGVIRIIAYVSGKPPPTVTWNMNERTLPQEATIETTAISSSMVIKNCQRSIIQGVY |
| | | SLLAKNEAGERKKTIIVDVLDVPGPVGTPFLAHNLTNESCKLTWFSPEDDGGSPITNYVI |
| | | EKRESDRRAWTPVTYTVTRQNATVQGLIQGKAYFFRIAAENSIGMGPFVETSEALVIREP |
| | | ITVPERPEDLEVKEVTKNTVTLTWNPPKYDGGSEIINYVLESRLIGTEKFHKVTNDNLLS |
| | | RKYTVKGLKEGDTYEYRVSAVNIVGQGKPSFCTKPITCKDELAPPTLHLDFRDKLTIRVG |
| | | EAFALTGRYSGKPKPKVSWFKDEADVLEDDRTHIKTTPATLALEKIKAKRSDSGKYCVVV |
| | | ENSTGSRKGFCQVNVVDRPGPPVGPVSFDEVTKDYMVISWKPPLDDGGSKITNYIIEKKE |
| | | VGKDVWMPVTSASAKTTCKVSKLLEGKDYIFRIHAENLYGISDPLVSDSMKAKDRFRVPD |
| | | APDQPIVTEVTKDSALVTWNKPHDGGKPITNYILEKRETMSKRWARVTKDPIHPYTKFRV |
| | | PDLLEGCQYEFRVSAENEIGIGDPSPPSKPVFAKDPIAKPSPPVNPEAIDTTCNSVDLTW |
| | | QPPRHDGGSKILGYIVEYQKVGDEEWRRANHTPESCPETKYKVTGLRDGQTYKFRVLAVN |
| | | AAGESDPAHVPEPVLVKDRLEPPELILDANMAREQHIKVGDTLRLSAIIKGVPFPKVTWK |
| | | KEDRDAPTKARIDVTPVGSKLEIRNAAHEDGGIYSLTVENPAGSKTVSVKVLVLDKPGPP |
| | | RDLEVSEIRKDSCYLTWKEPLDDGGSVITNYVVERRDVASAQWSPLSATSKKKSHFAKHL |
| | | NEGNQYLFRVAAENQYGRGPFVETPKPIKALDPLHPPGPPKDLHHVDVDKTEVSLVWNKP |
| | | DRDGGSPITGYLVEYQEEGTQDWIKFKTVTNLECVVTGLQQGKTYRFRVKAENIVGLGLP |
| | | DTTIPIECQEKLVPPSVELDVKLIEGLVVKAGTTVRFPAIIRGVPVPTAKWTTDGSEIKT |
| | | DEHYTVETDNFSSVLTIKNCLRRDTGEYQITVSNAAGSKTVAVHLTVLDVPGPPTGPINI |
| | | LDVTPEHMTISWQPPKDDGGSPVINYIVEKQDTRKDTWGVVSSGSSKTKLKIPHLQKGCE |
| | | YVFRVRAENKIGVGPPLDSTPTVAKHKFSPPSPPGKPVVTDITENAATVSWTLPKSDGGS |
| | | PITGYYMERREVTGKWVRVNKTPIADLKFRVTGLYEGNTYEFRVFAENLAGLSKPSPSSD |
| | | PIKACRPIKPPGPPINPKLKDKSRETADLVWTKPLSDGGSPILGYVVECQKPGTAQWNRI |
| | | NKDELIRQCAFRVPGLIEGNEYRFRIKAANIVGEGEPRELAESVIAKDILHPPEVELDVT |
| | | CRDVITVRVGQTIRILARVKGRPEPDITWTKEGKVLVREKRVDLIQDLPRVELQIKEAVR |
| | | ADHGKYIISAKNSSGHAQGSAIVNVLDRPGPCQNLKVTNVTKENCTISWENPLDNGGSEI |
| | | TNFIVEYRKPNQKGWSIVASDVTKRLIKANLLANNEYYFRVCAENKVGVGPTIETKTPIL |
| | | AINPIDRPGEPENLHIADKGKTFVYLKWRRPDYDGGSPNLSYHVERRLKGSDDWERVHKG |
| | | SIKETHYMVDRCVENQIYEFRVQTKNEGGESDWVKTEEVVYKEDLQKPVLDLKLSGVLTV |
| | | KAGDTIRLEAGVRGKPFPEVAWTKDKDATDLTRSPRVKIDTRADSSKFSLTKAKRSDGGK |
| | | YVVTATNTAGSFVAYATVNVLDKPGPVRNLKIVDVSSDRCTVCWDPPEDDGGCEIQNYIL |
| | | EKCETKRMVWSTYSATVLTPGTTVTRLIEGNEYIFRVRAENKIGTGPPTESKPVIAKTKY |
| | | DKPGRPDPPEVTKVSKEEMTVVWNPPEYDGGKSITGYFLEKKEKHSTRWVPVNKSAIPER |
| | | RMKVQNLLPDHEYQFRVKAENEIGIGEPSLPSRPVVAKDPIEPPGPPTNFRVVDTTKHSI |
| | | TLGWGKPVYDGGAPIIGYVVEMRPKIADASPDEGWKRCNAAAQLVRKEFTVTSLDENQEY |
| | | EFRVCAQNQVGIGRPAELKEAIKPKEILEPPEIDLDASMRKLVIVRAGCPIRLFAIVRGR |
| | | PAPKVTWRKVGIDNVVRKGQVDLVDTMAFLVIPNSTRDDSGKYSLTLVNPAGEKAVFVNV |
| | | RVLOTPGPVSDLKVSDVTKTSCHVSWAPPENDGOSQVTHYIVEKREADKKTWSTVTPEVK |
| | | KTSFHVTNLVPGNEYYFRVTAVNEYGPGVPTDVPKPVLASDPLSEPDPPRKLEVTEMTKN |
| | | SATLAWLPPLRDGGAKIDGYITSYREEEQPADRWTEYSVVKDLSLVVTGLKEGKKYKFRV |
| | | AARNAVGVSLPREAEGVYEAKEQLLPPKILMPEQITIKAGKKLRIEAHVYGKPHPTCKWK |
| | | KGEDEVVTSSHLAVHKADSSSILIIKDVTRKDSGYYSLTAENSSGTDTQKIKVVVMDAPG |
| | | PPQPPFDISDIDADACSLSWHIPLEDGGSNITNYIVEKCDVSRGDWVTALASVTKTSCRV |
| | | GKLIPGQEYIFRVRAENRFGISEPLTSPKMVAQFPFGVPSEPKNARVTKVNKDCIFVAWD |
| | | RPDSDGGSPIIGYLIERKERNSLLWVKANDTLVRSTEYPCAGLVEGLEYSFRIYALMKAG |
| | | SSPPSKPTEYVTARMPVDPPGKPEVIDVTKSTVSLIWARPKHDGGSKIIGYFVEACKLPG |
| | | DKWVRCNTAPHQIPQEEYTATGLEEKAQYQFRAIARTAVNISPPSEPSDPVTILAENVPP |
| | | RIDLSVAMKSLLTVKAGTNVCLDATVFGKPMPTVSWKKDGTLLKPAEGIKMAMQRNLCTL |
| | | ELFSVNRKDSGDYTITAENSSGSKSATIKLKVLDKPGPPASVKINKMYSDRAMLSWEPPL |
| | | EDGGSEITNYIVDKRETSRPNWAQVSAIVPITSCSVEKLIEGHEYQFRICAENKYGVGDP |
| | | VFTEPAIAKNPYDPPGRCDPPVISNITKDHMTVSWKPPADDGGSPITGYLLEKRETQAVN |
| | | WTKVNRKPIIERTLKATGLQEGTEYEFRVTAINKAGPGKPSDASKAAYARDPQYPPAPPA |
| | | FPKVYDTTRSSVSLSWGKPAYDGGSPIIGYLVEVKRADSDNWVRCNLPQNLQKTRFEVTG |
| | | LMEDTQYQFRVYAVNKIGYSDPSDVPDKHYPKDILIPPEGELDADLRKTLILRAGVTMRL |
| | | YVPVKGRPPPKITWSKPNVNLRDRIGLDJKSTDFDTFLRCENVNKYDAGKYILTLENSCG |
| | | KKEYTIVVKVLDTPGPPVNVTVKEISKDSAYVTWEPPIIDGGSPIINYVVQKRDAERKSW |
| | | STVTTECSKTSFRVANLEEGKSYFFRVFAENEYGIGDPGETRDAVKASQTPGPVVDLKVR |
| | | SVSKSSCSIGWKKPHSDGGSRIIGYVVDFLTEENKWQRVMKSLSLQYSAKDLTEGKEYTF |
| | | RVSAENENGEGTPSEITVVARDDVVAPDLDLKGLPDLCYLAKENSNFRLKIPIKGKPAPS |
| | | VSWKKGEDPLATDTRVSVESSAVNTTLIVYDCQKSDAGKYTITLKNVAGTKEGTISIKVV |
| | | GKPGIPTGPIKFDEVTAEAMTLKWAPPKDDGGSEITNYILEKRDSVNNKWVTCASAVQKT |
| | | TFRVTRLHEGMEYTFRVSAENKYGVGEGLKSEPIVARHPFDVPDAPPPPNIVDVRHDSVS |
| | | LTWTDPKKTGGSPITGYHLEFKERNSLLWKRANKTPIRMRDFKVTGLTEGLEYEFRVMAI |
| | | NLAGVGKPSLPSEPVVALDPIDPPGKPEVINITRNSVTLIWTEPKYDGGHKLTGYIVEKR |
| | | DLPSKSWMKANHVNVPECAFTVTDLVEGGKYEFRIRAKNTAGAISAPSESTETIICKDEY |
| | | EAPTIVLDPTIKDGLTIKAGDTIVLNAISILGKPLPKSSWSKAGKDIRPSDITQITSTPT |
| | | SSMLTIKYATRKDAGEYTITATNPFGTXVEHVKVTVLDVPGPPGPVEISNVSAEKATLTW |
| | | TPPLEDGGSPIKSYILEKRETSRLLWTVVSEDIQSCRHVATKLIQGNEYIFRVSAVNHYG |
| | | KGEPVQSEPVKMVDRFGPPGPPEKPEVSNVTKNTATVSWKRPVDDGGSEITGYHVERREK |
| | | KSLRWVRAIKTPVSDLRCKVTGLQEGSTYEFRVSAENRAGIGPPSEASDSVLMKDAAYPP |
| | | GPPSNPHVTDTTKKSASLAWGKPHYDGGLEITGYVVEHQKVGDEAWIKDTTGTALRITQF |
| | | VVPDLQTKEKYNFRISAINDAGVGEPAVIPDVEIVEREMAPDFELDAELRRTLVVRAGLS |
| | | IRIFVPIKGRPAPEVTWTKDNINLKNRANIENTESFTLLIIPECNRYDTGKFVMTIENPA |
| | | GKKSGFVNVRVLDTPGPVLNLRPTDITXDSVTLHWDLPLIDGGSRITNYIVEKREATRKS |
| | | YSTATTKCHKCTYKVTGLSEGCEYFFRVMAENEYGIGEPTETTEPVKASEAPSPPDSLNI |
| | | MDITKSTVSLAWPKPKHDGGSKITGYVIEAQRKGSDQWTHITTVKGLECVVRNLTEGEEY |
| | | TFQVMAVNSAGRSAPRESRPVIVKEQTMLPELDLRGIYQKLVIAKAGDNIKVEIPVLGRP |
| | | KPTVTWKKGDQILKQTQRVNFETTATSTILNINECVRSDSGPYPLTARNIVGEVGDVITI |
| | | QVHDIPGPPTGPIKFDEVSSDFVTTSWDPPENDGGVPISNYVVEMRQTDSTTWVELATTV |
| | | IRTTYKATRLTTGLEYQFRVKAQNRYGVGPGITSACIVANYPFKVPGPPGTPQVTAVTKD |
| | | SMTISWHEPLSDGGSPILGYHVERKERNGILWQTVSKALVPGNIFKSSGLTDGIAYEFRV |
| | | IAENMAGKSKPSKPSEPMLALDPIDPPGKPVPLNITRHTVTLKWAKPEYTGGFKITSYIV |
| | | EKRDLPNGRWLKANFSNILENEFTVSGLTEDAAYEFRVIAKNAAGAISPPSEPSDAITCR |
| | | DDVEAPKIKVDVKFKDTVILKAGEAFRLEADVSGRPPPTMEWSKDGKELEGTAKLEIKIA |
| | | DFSTNLVNKDSTRRDSGAYTLTATNPGGFAKHIFNVKVLDRPGPPEGPLAVTEVTSEKCV |
| | | LSWFPPLDDGGAKIDHYIVQKRETSRLAWTNVASEVQVTKLKVTKLLKGNEYIFRVMAVN |
| | | KYGVGEPLESEPVLAVNPYGPPDPPKNPEVTTITKDSMVVCWGHPDSDGGSEIINYIVER |
| | | RDKAGQRWIKCNKKTLTDLRYKVSGLTEGHEYEFRIMAENAAGISAPSPTSPFYKACDTV |
| | | FKPGPPGNPRVLDTSRSSISIAWNKPIYDGGSEITGYMVEIALPEEDEWQIVTPPAGLKA |
| | | TSYTITGLTENQEYKIRIYAMNSEGLGEPALVPGTPKAEDRMLPPEIELDADLRKVVTIR |
| | | ACCTLRLFVPIKGRPAPEVKWARDHGESLDKASIESTSSYTLLIVGNVNRFDSGKYILTV |
| | | ENSSGSKSAFVNVRVLDTPGPPQDLKVKEVTKTSVTLTWDPPLLDGGSKIKNYIVEKRES |
| | | TRKAYSTVATNCHKTSWKVDQLQEGCSYYFRVLAENEYGIGLPAETAESVKASERPLPPG |
| | | KITLMDVTRNSVSLSWEKPEHDGGSRILGYIVEMQTKGSDKWATCATVKVTEATITGLIQ |
| | | GEEYSFRVSAQNEKGISDPRQLSVPVIAKDLVIPPAFKLLFNTFTVLAGEDLKVDVPFIG |
| | | RPTPAVTWHKDNVPLKQTTRVNAESTENNSLLTIKDACREDVGHYVVKLTNSAGEAIETL |
| | | NVIVLDKPGPPTGPVKMDEVTADSITLSWGPPKYDGGSSINNYIVEKRDTSTTTWQIVSA |
| | | TVARTTIKACRLKTGCEYQFRIAAENRYGKSTYLNSEPTVAQYPFKVPGPPGTPVVTLSS |
| | | RDSMEVQWNEPISDGGSRVIGYHLERKERNSILWVKLNKTPIPQTKFKTTGLEEGVEYEF |
| | | RVSAENIVGIGKPSKVSECYVARDPCDPPGRPEAIIVTRNSVTLQWKKPTYDGGSKITGY |
| | | IVEKKELPEGRWMKASFTNIIDTHFEVTGLVEDHRYEFRVIARNAAGVFSEPSESTGAIT |
| | | ARDEVDPPRISMDPKYKDTIVVHAGESFKVDADIYGKPIPTIQWIKGDQELSNTARLEIK |
| | | STDFATSLSVKDAVRVDSGNYILKAKNVAGERSVTVNVKVLDRPGPPEGPVVISGVTAEK |
| | | CTLAWKPPLQDGGSDIINYIVERRETSRLVWTVVDANVQTLSCKVTKLLEGNEYTFRIMA |
| | | VNKYGVGEPLESEPVVAKNPFVVPDAPKAPEVTTVTKDSMIVVWERPASDGGSEILGYVL |
| | | EKRDKEGIRWTRCHKRLIGELRLRVTGLIENHDYEFRVSAENAAGLSEPSPPSAYQKACD |
| | | PIYKPGPPNNPKVIDITRSSVFLSWSKPIYDGGCEIQGYIVEKCDVSVGEWTMCTPPTGI |
| | | NKTNIEVEKLLEKHEYNFRICAINKAGVGEHADVPGPIIVEEKLEAPDIDLDLELRKIIN |
| | | IRAGGSLRLFVPIKGRPTPEVKWGKVDGEIRDAAIIDVTSSFTSLVLDNVNRYDSGKYTL |
| | | TLENSSGTKSAFVTVRVLDTPSPPVNLKVTEITKDSVSITWEPPLLDGGSKIKNYIVEKR |
| | | EATRKSYAAVVTNCHKNSWKIDQLQEGCSYYFRVTAENEYGIGLPAQTADPIKVAEVPQP |
| | | PGKITVDDVTRNSVSLSWTKPEHDGGSKIIQYIVEMQAKHSEKWSECARVKSLQAVITNL |
| | | TQGEEYLFRVVAVNEKGRSDPRSLAVPIVAKDLVIEPDVKPAFSSYSVQVGQDLKIEVPI |
| | | SGRPKPTITWTKDGLPLKQTTRINVTDSLDLTTLSIKETHKDDGGQYGITVANVVGQKTA |
| | | SIEIVTLDKPDPPKGPVKFDDVSAESITLSWNPPLYTGGCQITNYIVQKRDTTTTVWDVV |
| | | SATVARTTLKVTKLKTGTEYQFRIFAENRYGQSFALESDPIVAQYPYKEPGPPGTPFATA |
| | | ISKDSMVIQWHEPVNNGGSPVIGYHLERKERNSILWTKVNKTIIHDTQFKAQNLEEGIEY |
| | | EFRVYAENIVGVGKASKNSECYVARDPCDPPGTPEPIMVKRNEITLQWTKPVYDGGSMIT |
| | | GYIVEKRDLPDGRWMKASFTNVIETQFTVSGLTEDQRYEFRVIAKNAAGAISKPSDSTGP |
| | | ITAKDEVELPRISMDPKFRDTIVVNAGETFRLEADVHGKPLPTIEWLRGDKEIEESARCE |
| | | IKNTDFKALLIVKDAIRIDGGQYILRASNVAGSKSFPVNVKVLDRPGPPEGPVQVTGVTS |
| | | EKCSLTWSPPLQDGGSDISHYVVEKRETSRLAWTVVASEVVTNSLKVTKLLEGNEYVFRI |
| | | MAVNKYGVGEPLESAPVLMKNPFVLPGPPKSLENTTNIAKDSMTVCWNRPDSDGGSEHGY |
| | | IVEKRDRSGIRWIKCNKRRITDLRLRVTGLTEDHEYEFRVSAENAAGVGEPSPATVYYKA |
| | | CDPVFKPGPPTNAHIVDTIKNSITLAWGKPIYDGGSEILGYVVEICKADEEEWQIVTPQT |
| | | GLRVTRFEISKLTEHQEYKIRVCALNKVGLGEATSVPGTVKPEDKLEAPELDLDSELRKG |
| | | IVVRAGGSARIHIPFKGRPTPEITWSREEGEFTDKVQIEKGVNYTQLSIDNCDRNDAGKY |
| | | ILKLENSSGSKSAFVTVKVLDTPGPPQNLAVKEVRKDSAFLVWEPPIIDGGAKVKNYVID |
| | | KRESTRKAYANVSSKCSKTSFKVENLTCGAIYYFRVMAENEFGVGVPVETVDAVKAAEPP |
| | | SPPGKVTLTDVSQTSASLMWEKPEHDGGSRVLGYVVEMQPKGTEKWSIVAESKVCNAVVT |
| | | GLSSGQEYQFRVKAYNEKGKSDPRVLGVPVIAKDLTIQPSLKLPFNTYSIQAGEDLKIEI |
| | | PVIGRPRPNISWVKDGEPLKQTTRVNVEETATSTVLHIKEGNKDDFGKYTVTATNSAGTA |
| | | TENLSVIVLEKPGPPVGPVRFDEVSADFVVISWEPPAYTGGCQISNYIVEKRDTTTTIWH |
| | | MVSATVARTTIKITKLKTGTEYQFRIFAENRYGKSAPLDSKAVIVQYPFKEPGPPGTPFV |
| | | TSISKDQMLVQWHEPVNDGGTKIIGYHLEQKEKNSILWVKLNKTPIQDTKFKTTGLDEGL |
| | | EYEFKVSAENIVGIGKPSKVSECFVARDPCDPPGRPEAIVITRNNVTLKWKKPAYDGGSK |
| | | ITGYIVEKKDLPDGRWMKASFTNVLETEFTVSGLVEDQRYEFRVIARNAAGNFSEPSDSS |
| | | GAITARDEIDAPNASLDPKYKDVIVVHAGETFVLEADIRGKPIPDVVWSKDGKELEETAA |
| | | RMEIKSTIQKTTLVVKDCIRTDGGQYILKLSNVGGTKSIPITVKVLDRPGPPEGPLKVTG |
| | | VTAEKCYLAWNPPLQDGGANISHYIIEKRETSRLSWTQVSTEVQALNYKVTKLLPGNEYI |
| | | FRVMAVNKYGIGEPLESGPVTACNPYKPPGPPSTPEVSAITKDSMVVTWARPVDDGGTEI |
| | | EGYILEKRDKEGVRWTKCNKKTLTDLRLRVTGLTEGHSYEFRVAAENAAGVGEPSEPSVF |
| | | YRACDALYPPGPPSNPKVTDTSRSSVSLAWSKPIYDGGAPVKGYVVEVKEAAADEWTTCT |
| | | PPTGLQGKQITVTKLKENTEYNFRICAINSEGVGEPATLPGSVVAQERIEPPEIELDADL |
| | | RKVVVLRASATLRLFVTIKGRPEPEVKWEKAEGILTDRAQIEVTSSFTMLVIDNVTRFDS |
| | | GRYNLTLENNSGSKTAFVNVRVLDSPSAPVNLTIREVKKDSVTLSWEPPLIDGGAKITNY |
| | | IVEKRETTRKAYATITNNCTKTTFRIENLQEGCSYYFRVLASNEYGIGLPAETTTPVKVS |
| | | EPPLPPGRVTLVDVTRNTATIKWEKPESDGGSKITGYVVEMQTKGSEKWSTCTQVKTLEA |
| | | TISGLTAGEEYVFRVAAVNEKGRSDPRQLGVPVIARDIEIKPSVELPFHTFNVKAREQLK |
| | | IDVPFKGRPQATVNWRKDGQTLKETTRVNVSSSKTVTSLSIKEASKEDVGTYELCVSNSA |
| | | GSITVPITHVLDRPGPPGPIRIDEVSCDSITISVVNPPEYDGGCQISNYIVEKKETTSTT |
| | | WHIVSQAVARTSIKIVRLTTGSEYQFRVCAENRYGKSSYSESSAVVAEYPFSPPGPPGTP |
| | | KVVHATKSTMLVTWQVPVKDGGSRVIGYHLEYKERSSILWSKANKILIADTQMKVSGLDE |
| | | GLMYEYRVYAENIAGIGKCSKSCEPVPARDPCDPPGQPEVTNITRKSVSLKWSKPHYDGG |
| | | AKITGYIVERRELPDGRWLKCNYTNIQETYFEVTELTEDQRYEFRVFARNAADSVSEPSE |
| | | STGPIIVKDDVEPPRVMMDVKFRDVIVVKAGEVLKINADIAGRPLPVISWAKDGIEIEER |
| | | ARTEIISTDNHTLLTVKDCIRRDTGQYVLTLKNVAGTRSVAVNCKVLDKPGPPAGPLEIN |
| | | GLTAEKCSLSWGRPQEDGGADIDYYIVEKRETSHLAWTICEGELQMTSCKVTKLLKGNEY |
| | | IFRVTGVNKYGVGEPLESVAIKALDPFTVPSPPTSLEITSVTKESMTLCWSRPESDGGSE |
| | | ISGYIIERREKNSLRWVRVNKKPVYDLRVKSTGLREGCEYEYRVYAENAAGLSLPSETSP |
| | | LIRAEDPVFLPSPPSKPKIVDSGKTTITIAWVKPLFDGGAPTTGYTVEYKKSDDTDWKTS |
| | | IQSLRGTEYTISGLTTGAEYVFRVKSVNKVGASDPSDSSDPQIAKEREEEPLFDIDSEMR |
| | | KTLIVKAGASFTMTVPFRGRPVPNVLWSKPDTDLRTRAYVDTTDSRTSLTIENANRNDSG |
| | | KYTLTIQNVLSAASLTLVVKVLDTPGPPTNITVQDVTKESAVLSWDVPENDGGAPVKNYH |
| | | IEKREASKKAWVSVTNNCNRLSYKVTNLQEGAIYYFRVSGENEFGVGIPAETKEGVKITE |
| | | KPSPPEKLGVTSISKDSVSLTWLKPEHDGGSRIVHYVVEALEKGQKNWVKCAVAKSTHHV |
| | | VSGLRENSEYFFRVFAENQAGLSDPRELLLPVLIKEQLEPPEIDMKNFPSHTVYVRAGSN |
| | | LKVDIPISGKPLPKVTLSRDGVPLKATMRFNTEITAENLTINLKESVTADAGRYEITAAN |
| | | SSGTTKAFINIVVLDRPGPPTGPVVISDITEESVTLKWEPPKYDGGSQVTNYILLKRETS |
| | | TAVWTEVSATVARTMMKVMKLTTGEEYQFRIKAENRFGISDHIDSACVTVKLPYTTPGPP |
| | | STPWVTNVTRESITVGWHEPVSNGGSAVVGYHLEMKDRNSILWQKANKLVIRTTHFKVTT |
| | | ISAGLIYEFRVYAENAAGVGKPSHPSEPVLAIDACEPPRNVRITDISKNSVSLSWQQPAF |
| | | DGGSKITGYIVERRDLPDGRWTKASFTNVTETQFIISGLTQNSQYEFRVFARNAVGSISN |
| | | PSEVVGPITCIDSYGGPVIDLPLEYTEVVKYRAGTSVKLRAGISGKPAPTIEWYKDDKEL |
| | | QTNALVCVENTTDLASILIKDADRLNSGCYELKLRNAMGSASATIRVQILDKPGPPGGPI |
| | | EFKTVTAEKITLLWRPPADDGGAKITHYIVEKRETSRVVWSMVSEHLEECIITTTKIIKG |
| | | NEYIFRVRAVNKYGIGEPLESDSVVAKNAFVTPGPPGIPEVTKTTKNSMTVVWSRPIADG |
| | | GSDISGYFLEKRDKKSLGWFKVLKETIRDTRQKVTGLTENSDYQYRVCAVNAAGQGPFSE |
| | | PSEFYKAADPIDPPGPPAKIRIADSTKSSITLGWSKPVYDGGSAVTGYVVEIRQGEEEEW |
| | | TTVSTKGEVRTTEYVVSNLKPGVNYYFRVSAVNCAGQGEPIEMNEPVQAKDILEAPEIDL |
| | | DVALRTSVIAKAGEDVQVLIPFKGRPPPTVTWRKDEKNLGSDARYSIENTDSSSLLTIPQ |
| | | VTRNDTGKYILTIENGVGEPKSSTVSVKVLDTPAACQKLQVKHVSRGTVTLLWDPPLIDG |
| | | GSPIINYVIEKRDATKRTWSVVSHKCSSTSFKLIDLSEKTPFFFRVLAENEIGIGEPCET |
| | | TEPVKAAEVPAPIRDLSMKDSTKTSVILSWTKPDFDGGSVITEYVVERKGKGEQTWSHAG |
| | | ISKTCEIEVSQLKEQSVLEFRVFAKNEKGLSDPVTIGPITVKELIITPEVDLSDIPGAQV |
| | | TVRIGHNVHLELPYKGKPKPSISWLKDGLPLKESEFVRFSKTENKITLSIKNAKKEMGGK |
| | | YTVILDNAVCRIAVPITVITLGPPSKPKGPIRFDEIKADSVILSWDVPEDNGGGEITCYS |
| | | IEKRETSQTNWKMVCSSVARTTFKVPNLVKDAEYQFRVRAENRYGVSQPLVSSIIVAKHQ |
| | | FRIPGPPGKPVIYNVTSDGMSLTWDAPVYDGGSEVTGFHVEKKERNSILWQKVNTSPISG |
| | | REYRATGLVEGLDYQFRVYAENSAGLSSPSDPSKFTLAVSPVDPPGTPDYIDVTRETITL |
| | | KWNPPLRDGGSKIVGYSIEKRQGNERWVRCNFTDVSECQYTVTGLSPGDRYEFRIIARNA |
| | | VGTISPPSQSSGIIMTRDENVPPIVEFGPEYFDGLIIKSGESLRIKALVQGRPVPRVTWF |
| | | KDGVEIEKRMNMEITDVLGSTSLFVRDATRDHRGVYTVEAKNASGSAKAEIKVKVQDTPG |
| | | KVVGPIRFTNITGEKMTLWWDAPLNDGCAPITHYIIEKRETSRLAWALIEDKCEAQSYTA |
| | | IKLINGNEYQFRVSAVNKFGVGRPLDSDPVVAQIQYTVPDAPGIPEPSNITGNSITLTWA |
| | | RPESDGGSEIQQYILERREKKSTRWVKVISKRPISETRFKVTGLTEGNEYEFHVMAENAA |
| | | GVGPASGISRLIKCREPVNPPGPPTVVKVTDTSKTTVSLEWSKPVFDGGMEIIGYIIEMC |
| | | KADLGDWHKVNAEACVKTRYTVTDLQAGEEYKFRVSAINGAGKGDSCEVTGTIKAVDRLT |
| | | APELDIDANFKQTHVVRAGASIRLFIAYQGRPTPTAVWSKPDSNLSLRADIHTTDSFSTL |
| | | TVENCNRNDAGKYTLTVENNSGSKSITFTVKVLDTPGPPGPrTFKDVTRGSATLMWDAPL |
| | | LDGGARIHHYVVEKREASRRSWQVISEKCTRQIFKVNDLAEGVPYYFRVSAVNEYGVGEP |
| | | YEMPEPIVATEQPAPPRRLDVVDTSKSSAVLAWLKPDHDGGSRITGYLLEMRQKGSDFWV |
| | | EAGHTKQLTFTVERLVEKTEYEFRVKAKNDAGYSEPREAFSSVIIKEPQIEPTADLTGIT |
| | | NQLITCKAGSPFTIDVPISGRPAPKVTWKLEEMRLKETDRVSITTTKDRTTLTVKDSMRG |
| | | DSGRYFLTLENTAGVKTFSVTVVVIGRPGPVTGPIEVSSVSAESCVLSWGEPKDGGGTEI |
| | | TNYIVEKRESGTTAWQLVNSSVKRTQIKVTHLTKYMEYSFRVSSENRFGVSKPLESAPII |
| | | AEHPFVPPSAPTRPEVYHVSANAMSIRWEEPYHDGGSKIIGYWVEKKERNTILWVKENKV |
| | | PCLECNYKVTGLVEGLEYQFRTYALNAAGVSKASEASRPIMAQNPVDAPGRPEVTDVTRS |
| | | TVSLIWSAPAYDGGSKVVGYIIERKPVSEVGDGRWLKCNYTIVSDNFFTVTALSEGDTYE |
| | | FRVLAKNAAGVISKGSESTGPVTCRDEYAPPKAELDARLHGDLVTIRAGSDLVLDAAVGG |
| | | KPEPKIIWTKGDKELDLCEKVSLQYTGKRATAVIKFCDRSDSGKYTLTVKNASGTKAVSV |
| | | MVKVLDSPGPCGKLTVSRVTQEKCTLAWSLPQEDGGAEITHYIVERRETSRLNWVIVEGE |
| | | CPTLSYVVTRLIKNNEYIFRVRAVNKYGPGVPVESEPIVARNSFTIPSPPGIPEEVGTGK |
| | | EHIIIQWTKPESDGGNEISNYLVDKREKKSLRWTRVNKDYVVYDTRLKVTSLMEGCDYQF |
| | | RVTAVNAAGNSEPSEASNFISCREPSYTPGPPSAPRVVDTTKHSISLAWTKPMYDGGTDI |
| | | VGYVLEMQEKDTDQWYRVHTNATIRNTEFTVPDLKMGQKYSFRVAAVNVKGMSEYSESIA |
| | | EIEPVERIEIPDLELADDLKKTVTIRAGASLRLMVSVSGRPPPVITWSKQGIDLASRAII |
| | | DTTESYSLLIVDKVNRYDAGKYTIEAENQSGKKSATVLVKVYDTPGPCPSVKVKEVSRDS |
| | | VTITWEIPTIDGGAPVNNYIVEKREAAMRAFKTVTTKCSKTLYRISGLVEGTMYYFRVLP |
| | | ENIYGIGEPCETSDAVLVSEVPLVPAKLEVVDVTKSTVTLAWEKPLYDGGSRLTGYVLEA |
| | | CKAGTERWMKVVTLKPTVIEHTVTSLNEGEQYLFRIRAQNEKGVSEPRETVTAVTVQDLR |
| | | VLPTIDLSTMPQKTIHVPAGRPVELVIPIAGRPPPAASWFFAGSKLRESERVTVETHTKV |
| | | AKLTIRETTIRDTGEYTLELKNVTGTTSETIKVIILDKPGPPTGPIKIDEIDATSITISW |
| | | EPPELDGGAPLSGYVVEQRDAHRPGWLPVSESVTRSTFKFTRLTEGNEYVFRVAATNRFG |
| | | IGSYLQSEVIECRSSIRIPGPPETLQIFDVSRIIMTLTWYPPEDDGGSQVTGYIVERKEV |
| | | RADRWVRVNKVPVTMTRYRSTGLTEGLEYEHRVTAINARGSGKPSRPSKPIVAMDPIAPP |
| | | GKPQNPRVTDTTRTSVSLAWSVPEDEGGSKVTGYLIEMQKVDQHEWTKCNTTPTKIREYT |
| | | LTHLPQGAEYRFRVLACNAGGPGEPAEVPGTVKVTEMLEYPDYELDERYQEGIFVRQGGV |
| | | IRLTIPIKGKPFPICKWTKEGQDISKRAMIATSETHTELVIKEADRGDSGTYDLVLENKC |
| | | GKKAVYIKVRVIGSPNSPEGPLEYDDIQVRSVRVSWRPPADDGGADILGYILERREVPKA |
| | | AWYTIDSRVRGTSLVVKGLKENVEYHFRVSAENQFGISKPLKSEEPVTPKTPLNPPEPPS |
| | | NPPEVLDVTKSSVSLSWSRPKDDGGSRVTGYYIERKETSTDKWVRHNKTQITTTMYTVTG |
| | | LVPDAEYQFRIIAQNDVGLSETSPASEPVVCKDPFDKPSQPGELEILSISKDSVTLQWEK |
| | | PECDGGKEILGYWVEYRQSGDSAWKKSNKERIKDKQFTIGGLLEATEYEFRVFAENETGL |
| | | SRPRRTAMSIKTKLTSGEAPGIRKEMKDVTTKLGEAAQICQIVGRPLPDIKWYIIFGKEL |
| | | IQSRKYKMSSDGRTHTLTVMTEEQEDEGVYTCIATNEVGEVETSSKLLLQATPQFMPGYP |
| | | LKEKYYGAVGSTLRLHVMYIGRPVPAMTWFHGQKLLQNSENITIENTEMYTHLVMKNVQR |
| | | KTHAGKYKVQLSNVFGTVDAILDVEIQDKPDKPTGPIVIEALLKNSAVISWKPPADDGGS |
| | | WITNYVVEKCEAKEGAEWQLVSSAISVTTCRIVNLTENAGYYFRVSAQNTFGISDPLEVS |
| | | SVVIIKSPFEKPGAPGKPTITAVTKDSCVVAWKPPASDGGAKIRNYYLEKREKKQNKWIS |
| | | VTTEEIRETVFSVKNLIEGLEYEFRVKCENLGGESEWSEISEPITPKSDVPIQAPHFKEE |
| | | LRNLNVRYQSNATLVCKVTGHPKPIVKWYRQGKEIIADGLKYRIQEFKGGYHQLIIASVT |
| | | DDDATVYQVRATNQGGSVSGTASLEVEVPAKIHLPKTLEGMGAVHALRGEVVSIKIPFSG |
| | | KPDPVITWQKGQDLIDNNGHYQVIVTRSFTSLVFPNGVERKDAGFYVVCAKNRFGIDQKT |
| | | VELDVADVPDPPRGVKVSDVSRDSVNLTWTEPASDGGSKITNYIVEKCATTAERWLRVGQ |
| | | ARETRYTVINLFGKTSYQFRVIAENKFGLSKPSEPSEPTITKEDKTRAMNYDEEVDETRE |
| | | VSMTKASHSSTKELYEKYMIAEDLGRGEFGIVHRCVETSSKKTYMAKFVKVKGTDQVLVK |
| | | KEISILNIARHRNILHLHESFESMEELVMIFEFISGLDIFERINTSAFELNEREIVSYVH |
| | | QVCEALQFLHSHNIGHFDIRPENIIYQTRRSSTIKIIEFGQARQLKPGDNFRLLFTAPEY |
| | | YAPEVHQHDVVSTATDMWSLGTLVYVLLSGINPFLAETNQQIIENIMNAEYTFDEEAFKE |
| | | ISIEAMDFVDRLLVKERKSRMTASEALQHPWLKQKIERVSTKVIRTLKHRRYYHTLIKKD |
| | | LNMVVSAARISCGGAIRSQKGVSVAKVKVASIEIGPVSGQIMHAVGEEGGHVKYVCKIEN |
| | | YDQSTQVTWYFGVRQLENSEKYEITYEDGVAILYVKDITKLDDGTYRCKVVNDYGEDSSY |
| | | AELFVKGVREVYDYYCRRTMKKfKRRTDTMRLLERPPEFTLPLYNKTAYVGENVRFGVTI |
| | | TVHPEPHVTWYKSGQKIKPGDNDKKYTFESDKGLYQLTINSVTTDDDAEYTVVARNKYGE |
| | | DSCKAKLTVTLHPPPTDSTLRPMFKRLLANAECQEGQSVCFEIRVSGIPPPTLKWEKDGQ |
| | | PLSLGPNIEIIHEGLDYYALHIRDTLPEDTGYYRVTATNTAGSTSCQAHLQVERLRYKKQ |
| | | EFKSKEEHERHVQKQIDKTLRMAEILSGTESVPLTQVAKEALREAAVLYKPAVSTKTVKG |
| | | EFRLEIEEKKEERKLRMPYDVPEPRKYKQTTIEEDQRIKQFVPMSDMKWYKKIRDQYEMP |
| | | GKLDRVVQKRPKRIRLSRWEQFYVMPLPRITDQYRPKWRIPKLSQDDLEIVRPARRRTPS |
| | | PDYDFYYRPRRRSLGDISDEELLLPIDDYLAMKRTEEERLRLEEELELGFSASPPSRSPP |
| | | HFELSSLRYSSPQAHVKVEETRKDFRYSTYHIPTKAEASTSYAELRERHAQAAYRQPKQR |
| | | QRIMAEREDEELLRPVTTTQHLSEYKSELDFMSKEEKSRKKSRRQREVTEITEIEEEYEI |
| | | SKHAQRESSSSASRLLRRRRSLSPTYIELMRPVSELIRSRPQPAEEYEDDTERRSPTPER |
| | | TRPRSPSPVSSERSLSRFERSARFDIFSRYESMKAALKTQKTSERKYEVLSQQPFTLDHA |
| | | PRITLRMRSHRVPCGQNTRFILNVQSKPTAEVKWYHNGVELQESSKIHYTNTSGVLTLEI |
| | | LDCHTDDSGTYRAVCTNYKGEASDYATLDVTGGDYTTYASQRRDEEVPRSVFPELTRTEA |
| | | YAVSSFKKTSEMEASSSVREVKSQMTETRESLSSYEHSASAEMKSAALEEKSLEEKSTTR |
| | | KIKTTLAARILTKPRSMTVYEGESARFSCDTDGEPVPTVTWLRKGQVLSTSARHQVTTTK |
| | | YKSTFEISSVQASDEGNYSVVVENSEGKQEAEFTLTIQKARVTEKAVTSPPRVKSPEPRV |
| | | KSPEAVKSPKRVKSPEPSHPKAVSPTETKPTPTEKVQHLPVSAPPKITQFLKAEASKEIA |
| | | KLTCVVESSVLRAKEVTWYKDGKKLKENGHFQFHYSADGTYELKINNLTESDQGEYVCEI |
| | | SGEGGTSKTNLQFMGQAFKSIHEKVSKISETKKSDQKTTESTVTRKTEPKAPEPISSKPV |
| | | IVTGLQDTTVSSDSVAKFAVKATGEPRPTAIWTKDGKAITQGGKYKLSEDKGGFFLEIHK |
| | | TDTSDSGLYTCTVKNSAGSVSSSCKLTIKAIKDTEAQKVSTQKTSEITPQKKAVVQEEIS |
| | | QKALRSEEIKMSEAKSQEKLALKEEASKVLISEEVKKSAATSLEKSIVHEEITKTSQASE |
| | | EVRTHAEIKAFSTQMSINEGQRLVLKANIAGATDVKWVLNGVELTNSEEYRYGVSGSDQT |
| | | LTIKQASHRDEGILTCISKTKEGIVKCQYDLTLSKELSDAPAFISQPRSQNINEGQNVLF |
| | | TCEISGEPSPEIEWFKNNLPISISSNVSISRSRNVYSLEIRNASVSDSGKYTIKAKNFRG |
| | | QCSATASLMVLPLVEEPSREVVLRTSGDTSLQGSFSSQSVQMSASKQEASFSSFSSSSAS |
| | | SMTEMKFASMSAQSMSSMQESFVEMSSSSFMGISNMTQLESSTSKMLKAGIRGIPPKIEA |
| | | LPSDISIDEGKVLTVACAFTGEPTPEVTWSCGGRKIHSQEQGRFHIENTDDLTTLHMDV |
| | | QKQDGGLYTLSLGNEFGSDSATVNIHIRSI |
| 37 | CAP-Gly domain-containing | MSMLKPSGLKAPTKILKPGSTALKTPTAVVAPVEKTISSEKASSTPSSETQEEFVDDFRV |
| | linker protein 1 (CLIP1) | GERVWVNGNKPGFIQFLGETQFAPGQWAGIVLDEPIGKNDGSVAGVRYFQCEPLKGIFTR |
| | | PSKLTRKVQAEDEANGLQTTPASRATSPLCTSTASMVSSSPSTPSNIPQKPSQPAAKEPS |
| | | ATPPISNLTKTASESISNLSEAGSIKKGQRELKIGDRVLVGGTKAGVVRFLGETDFAKGE |
| | | WCGVELDEPLGKNDGAVAGTRYFQCQPKYGLFAPVHKVTKIGFPSTTPAKAKANAVRRVM |
| | | ATTSASLKRSPSASSLSSMSSVASSVSSRPSRTGLLTETSSRYARKISGTTALQEALKEK |
| | | QQHIEQLLAERDLERAEVAKATSHVGEIEQELALARDGHDQHVLELEAKMDQLRTMVEAA |
| | | DREKVELLNQLEEEKRKVEDLQFRVEEESITKGDLEQKSQISEDPENTQTKLEHARIKEL |
| | | EQSLLFEKTKADKLQRELEDTRVATVSEKSRIMELEKDLALRVQEVAELRRRLESNKPAG |
| | | DVDMSLSLLQEISSLQEKLEVTRTDHQREITSLKEHFGAREETHQKEIKALYTATEKLSK |
| | | ENESLKSKLEHANKENSDVIALWKSKLETAIASHQQAMEELKVSFSKGLGTETAEFAELK |
| | | TQIEKMRLDYQHEIENLQNQQDSERAAHAKEMEALRAKLMKVIKEKENSLEAIRSKLDKA |
| | | EDQHLVEMEDTLNKLQEAEIKVKELEVLQAKCNEQTKVIDNFTSQLKATEEKLLDLDALR |
| | | KASSEGKSEMKKLRQQLEAAEKQIKHLEIEKNAESSKASSITRELQGRELKLTNLQENLS |
| | | EVSQVKETLEKELQILKEKFAEASEEAVSVQRSMQETVNKLHQKEEQFNMLSSDLEKLRE |
| | | NLADMEAKFREKDEREEQLIKAKEKLENDIAEIMKMSGDNSSQLTKMNDELRLKERDVEE |
| | | LQLKLTKANENASFLQKSIEDMTVKAEQSQQEAAKKHEEEKKELERKLSDLEKKMETSUN |
| | | QCQELKARYERATSETKTKHEEILQNLQKTLLDTEDKLKGAREENSGLLQELEELRKQAD |
| | | KAKAAQTAEDAMQIMEQMTKEKTETLASLEDTKQTNAKLQNELDTLKENNLKNVEELNKS |
| | | KELLTVENQKMEEFRKEIETLKQAAAQKSQQLSALQEENVKLAEELGRSRDEVTSHQKLE |
| | | EERSVLKNQLLEMKKRESKFIKDADEEKASLQKSISITSALLTEKDAELEKLRNEVTVLR |
| | | GENASAKSLHSVVQTLESDKVKLELKVKNLELQLKENKRQLSSSSGNTDTQADEDERAQE |
| | | SQIDFLNSVIVDLQRKNQDLKMKVEMMSEAALNGNGDDLNNYDSDDQEKQSKKKPRLFCD |
| | | ICDCFDLHDTEDCPTQAQMSEDPPHSTHHGSRGEERPYCEICEMFGHWATNCNDDETF |
| 38 | Mitotic checkpoint | MAAVKKEGGALSEAMSLEGDEWELSKENVQPLRQGRIMSTLQGALAQESACNNTLQQQKR |
| | serine/threonine-protein | AFEYEIRFYTGNDPLDVWDRYISWTEQNYPQGGKESNMSTLLERAVEALQGEKRYYSDPR |
| | kinase BUB1 beta (BUB1B) | FLNLWLKLGRLCNEPLDMYSYLHNQGIGVSLAQFYISWAEEYEARENFRKADAIFQEGIQ |
| | | QKAEPLERLQSQHRQFQARVSRQTLLALEKEEEEEVFESSVPQRSTLAELKSKGKKTARA |
| | | PIIRVGGALKAPSQNRGLQNPFPQQMQNNSRITVFDENADEASTAELSKPTVQPWIAPPM |
| | | PRAKENELQAGPWNTGRSLEHRPRGNTASLIAVPAVLPSFTPYVEETARQPVMTPCKIEP |
| | | SINHILSTRKPGKEEGDPLQRVQSHQQASEEKKEKMMYCKEKIYAGVGEFSFEEIRAEVF |
| | | RKKLKEQREAELLTSAEKRAEMQKQIEEMEKKLKEIQTTQQERTGDQQEETMPTKETTKL |
| | | QIASESQKIPGMTLSSSVCQVNCCARETSLAENIWQEQPHSKGPSVPFSIFDEFLLSEKK |
| | | NKSPPADPPRVLAQRRPLAVLKTSESITSNEDVSPDVCDEFTGIEPLSEDAIITGFRNVT |
| | | ICPNPEDTCDFARAARFVSTPFHEIMSLKDLPSDPERLLPEEDLDVKTSEDQQTACGTIY |
| | | SQTLSIKKLSPIIEDSREATHSSGFSGSSASVASTSSIKCLQIPEKLELTNETSENPTQS |
| | | PWCSQYRRQLLKSLPELSASAELCIEDRPMPKLEIEKEIELGNEDYCIKREYLICEDYKL |
| | | FWVAPRNSAELTVIKVSSQPVPWDFYINLKLKERLNEDFDHFCSCYQYQDGCIVWHQYIN |
| | | CFTLQDLLQHSEYITHEITVLIIYNLLTIVEMLHKAEIVHGDLSPRCLILRNRIHDPYDC |
| | | NKNNQALKIVDFSYSVDLRVQLDVFTLSGFRTVQILEGQKILANCSSPYQVDLFGIADLA |
| | | HLLLFKEHLQVFWDGSFWKLSQNISELKDGELWNKFFVRILNANDEATVSVLGELAAEMN |
| | | GVFDTTFQSHLNKALWKVGKLTSPGALLFQ |
| 39 | Rho guanine nucleotide | MEDFARGAASPGPSRPGLVPVSIIGAEDEDFENELETNSEEQNSQFQSLEQVKRRPAHLM |
| | exchange factor 1 | ALLQHVALQFEPGPLLCCLHADMLGSLGPKEAKKAFLDFYHSFLEKTAVLRVPVPPNVAF |
| | (ARHGEF1) | ELDRTRADLISEDVQRRFVQEVVQSQQVAVGRQLEDFRSKRLMGMTPWEQELAQLEAWVG |
| | | RDRASYEARERHVAERLLMHLEEMQHTISTDEEKSAAVVNAIGLYMRHLGVRTKSGDKKS |
| | | GRNFFRKKVMGNRRSDEPAKTKKGLSSILDAARWNRGEPQVPDFRHLKAEVDAEKPGATD |
| | | RKGGVGMPSRDRNIGAPGQDTPGVSLHPLSLDSPDREPGADAPLELGDSSPQGPMSLESL |
| | | APPESTDEGAETESPEPGDEGEPGRSGLELEPEEPPGWRELVPPDTLHSLPKSQVKRQEV |
| | | ISELLVTEAAUVRMLRVLHDIFFQPMAECLFFFLEELQNIFPSLDELIEVHSLFLDRIMK |
| | | QEAESRPRCRRLQLKDMIPTEMQRLTKYPLLLQSIGQNTEEPTEREKVELAAECCREILH |
| | | HVNQAVRDMEDLLRLKDYQRRLDLSHLRQSSDPMLSEFKNLDITKKKLVHEGPLTWRVTK |
| | | DKAVEVHVLLLDDLLLLLQRQDERLLLKSHSRTLTPTPDGKTMLRPVLRLTSAMTREVAT |
| | | DHKAFYVLFTWDQEAQIYELVAQTVSERKNWCALITETAGSLKVPAPASRPKPRPSPSST |
| | | REPLLSSSENGNGGRETSPADARTERILSDLLPFCRPGPEGQLAATALRKVLSLKQLLFP |
| | | AEEDNGAGPPRDGDGVPGGGPLSPARTQEIQENLLSLEETMKQLEELEEEFCRLRPLLSQ |
| | | LGGNSVPQPGCT |
| 40 | Titin (TTN) | MTTQAPTFTQPLQSVVVLEGSTATFEAHISGFPVPEVSWFRDGQVISTSTLPGVQISFSD |
| | | GRAKLTIPAVTKANSGRYSLKATNGSGQATSTAELLVKAETAPPNFVQRLQSMTVRQGSQ |
| | | VRLQVRVTGIPTPVVKFYRDGAEIQSSLDFQISQEGDLYSLLIAEAYPEDSGTYSVNATN |
| | | SVGRATSTAELLVQGEEEVPAKKTKTIVSTAQISESRQTRIEKKIEAHFDARSIATVEMV |
| | | IDGAAGQQLPHKTPHRIPPKPKSRSPTPPSIAAKAQLARQQSPSPIRHSPSPVRHVRAPT |
| | | PSPVRSVSPAARISTSPIRSVRSPLLMRKTQASTVATGPEVPPPWKQEGYVASSSEAEMR |
| | | ETTLTTSTQIRTEERWEGRYGVQEQVTISGAAGAAASVSASASYAAEAVATGAKEVKQDA |
| | | DKSAAVATVVAAVDMARVREPVISAVEQTAQRTTTTAVMIQPAQEQVRKEAEKTAVTKVV |
| | | VAADKAKEQELKSRTKEVITTKQEQMHVnTHEQIRKETEKTFVPKWISAAKAKEQETRIS |
| | | EEETKKQKQVTQEAIRQETEITAASMVWATAKSTKLETVPGAQEETTTQQDQMHLSYEIC |
| | | IMKETRKTVVPKVIVATPKVKEQDLVSRGREGITTKREQVQITQEKMRKEAEKTALSTIA |
| | | VATAKAKEQETILRTRETMATRQEQIQVTHGKVDVGKKAEAVATVVAAVDQARVREPREP |
| | | CHLEESYAQQTTLEYGYKERISAAKVAEPPQRPASEPHVVPKAVKPRVIQAPSETHIKTT |
| | | DQKGMHISSQIKKTTDLTTERLVHVDKRPRTASPHFTVSKISVPKTEHGYEASIAGSAIA |
| | | TLQKELSATSSAQKITKSVKAPTVKPSETRVRAEPTPLPQFPFADTPDTYKSEAGVEVKK |
| | | EVGVSITGTTVREERFEVLHGREAKVTETARVPAPVEIPVTPPTLVSGLKNVTVIEGESV |
| | | TLECHISGYPSPTVTWYREDYQIESSIDFQITFQSGIARLMIREAFAEDSGRFTCSAVNE |
| | | AGTVSTSCYLAVQVSEEFEKETTAVTEKFTTEEKRFVESRDVVMTDTSLTEEOAGPGEPA |
| | | APYFITKPWQKLVEGGSVVFGCQVGGNPKPHVYVVKKSGVPLTTGYRYKVSYNKQTGECK |
| | | LVISMTFADDAGEYTIVVRNKHGETSASASLLEEADYELLMKSQQEMLYQTQVTAFVQEP |
| | | KVGETAPGFVYSEYEKEYEKEQALIRKKMAKDTVVVRTYVEDQEFHISSFEERIIKEIEY |
| | | RIIKTTLEELLEEDGEEKMAVDISESEAVESGFDLRIKNYRILEGMGVTFHCKMSGYPLP |
| | | KIAWYKDGKRIKHGERYQMDFLQDGRASLRIPVVLPEDEGIYTAFASNIKGNAICSGKLY |
| | | VEPAAPLCAPTYIPTLEPVSRIRSLSPRSVSRSPIRMSPARMSPARMSPARMSPARMSPG |
| | | RRLEETDESQLERLYKPVFVLKPVSFKCLEGQTARFDLKVVGRPMPETFWrHDGQQIVND |
| | | YTHKVVIKEDGTQSLIIVPATPSDSGEWTVVAQNRAGRSSISVILTVEAVEHQVKPMFVE |
| | | KLKNVNIKEGSQLEMKVUATGNPNPDIVWLKNSDIIVPHKYPKIRIEGTKGEAALKIDST |
| | | VSQDSAVVYTATAINKAGRDTTRCKVNVEVEFAEPEPERKLNPRGTYRAKEIAAPELEPL |
| | | HLRYGQEQWEEGDLYDKEKQQKPFFKKKLTSLRLKRFGPAHFECRLTPIGDPTMVVEWLH |
| | | DGKPLEAANRLRMINEFGYCSLDYGVAYSRDSGIITCRATNKYGTDHTSATLIVKDEKSL |
| | | VEESQLPEGRKGLQRTEELERMAHEGALTGVTTDQKEKQKPDIVLYPEPVRVLEGETARF |
| | | RCRVTGYPQPKVNWYLNGQLIRKSKRFRVRYDGIHYLDIVITKSYDTGEVKVTAENPEGV |
| | | IEHKVKLEIQQREDFRSVLRRAPFPRPEFHVHEPGKLQFEVQKVDRPVDTTETKEVVKLK |
| | | RAERITHEKVPEESEELRSKFKRRTEEGYYEAITAVELKSRKKDESYEELLRKTKDELLH |
| | | WTKELTEEEKXALAEEGKITIPTFKPDKIELSPSMEAPKIFERIQSQTVGQGSDAHFRVR |
| | | VVGKPDPECEWYKNGVKIERSDRIYWYWPEDNVCELVIRDVTAEDSASIMVKAINIAGET |
| | | SSHAFLLVQAKQLITFTQELQDVVAKEKDTMATFECETSEPFVKVKWYKDGMEVHEGDKY |
| | | RMHSDRKVHFLSILTIDTSDAEDYSCVLVEDENVKTTAKLIVEGAVVEFVKELQDIEVPE |
| | | SYSGELECIVSPENIEGKWYHNDVELKSNGKYTITSRRGRQNLTVKDVTKEDQGEYSFVI |
| | | DGKKTTCKLKMKPRPIAILQGLSDQKVCEGDIVQLEVKVSLESVEGVWMKDGQEVQPSDR |
| | | VHIVIDKQSHMLLIEDMTKEDAGNYSFTIPALGLSTSGRVSVYSVDVITPLKDVNVIEGT |
| | | KAVLECKVSVPDVTSVKWYLNDEQIKPDDRVQAIVKGTKQRLVINRTHASDEGPYKLIVG |
| | | RVETNCNLSVEKIKIIRGLRDLTCTETQNVVFEVELSHSGIDVLWNFKDKEIKPSSKYKI |
| | | EAHGKIYKLTVLNMMKDDEGKYTFYAGENITSGKLTVAGGAISKPLTDQTVAESQEAVFE |
| | | CEVANPDSKGEWLRDGKHLPLTNNIRSESDGHKRRLIIAATKLDDIGEYTYKVATSKTSA |
| | | KLKVEAVKIKKTLKNLTVTETQDAVFTVELTHPNVKGVQWIKNGVVLESNEKYAISVKGT |
| | | IYSLRIKNCAIVDESVYGFRLGRLGASARLHVETVKIIKKPKDVTALENATVAFEVSVSH |
| | | DTVPVKWFHKNVEIKPSDKHRLVSERKVUKLMLQNISPSDAGEYTAVVGQLECKAKLFVE |
| | | TLHITKTMKNIEVPETKTASFECEVSHFNVPSMVVLKNGVEIEMSEKFKIVVQGKLHQLI |
| | | MNTSTEDSAEYTFVCGNDQVSATLTVTPIMITSMLKDINAEEKDTITFEVTVNYEGISYK |
| | | WLKNGVEIKSTDKCQMRTKKLTHSLNIRNVHFGDAADYTFVAGKATSTATLYVEARHIEF |
| | | RKIHKDIKVLEKKRAMFECEVSEPDITVQWMKDDQELQITDRIKIQKEKYVHRLLIPSTR |
| | | MSOAGKYTVVAGGNVSTAKLFYEGRDVRIRSIKKEVQVIEKQRAVVEFEVNEDDVDAHWY |
| | | KDGIEINFQVQERHKYVVERRIHRMFISETRQSDAGEYTFVAGRNRSSVTLYVNAPEPPQ |
| | | VLQELQPVTVQSGKPARFCAVISGRPQPKISWYKEEQLLSTGFKCKFLHDGQEYTLLLIE |
| | | AFPEUAAVYTCEAKNDYGVATTSASLSVEVPEVVSPDQEMPVYPPAITTPLQDTVTSEGQ |
| | | PARFQCRVSGTDLKVSWYSKDKKIKPSRFFRMTQFEDTYQLEIAEAYPEDEGTYTFVASN |
| | | AVGQVSSTANLSLEAPESILHERIEQEIEMEMKEFSSSFLSAEEEGLHSAELQLSKINET |
| | | LELLSESPVYSTKFDSEKEGTGPIFIKEVSNADISMGDVATLSVTVIGIPKPKIQWFFNG |
| | | VLLTPSADYKFVFDGDDHSLIILFTKLEDEGEYTCMASNOYGKTICSAYLKINSKGEGKK |
| | | DTETESAVAKSLEKLGGPCPPHFLKELKPIRCAQGLPAIFEYTWGEPAPTVTVVFKENKQ |
| | | LCTSVYYTIIHNPNGSGTFIVNDPQREDSGLYICKAENMLGESTCAAELLVLLEDTDMTD |
| | | TPCKAKSTPEAPEDFPQTPLKGPAVEALDSEQEIATFVKDTILKAALITEENQQLSYEHI |
| | | AKANELSSQLPLGAQELQSILEQDKLTPESTREFLCINGSIHFQPLKEPSPNLQLQIVQS |
| | | QKTFSKEGILMPEEPETQAVLSDTEKIFPSAMSIEQINSLTVEPLKTLLAEPEGNYPQSS |
| | | IEPPMHSYLTSVAEEVLSPKEKTVSDTNREQRVTLQKQEAQSALILSQSLAEGHVESLQS |
| | | PDVMISQVNYEPLVPSEHSCTEGGKILIESANPLENAGQDSAVRIEEGKSLRFPLALEEK |
| | | QVLLKEEHSDNVVMPPDQIIESKREPVAIKKVQEVQGRDLLSKESLLSGIPEEQRLNLKI |
| | | QICRALQAAVASEQPGLFSEWLRNIEKVEVEAVNITQEPRHIMCMYLVTSAKSVTEEVTI |
| | | ILEDVDPQMANLKMELRDALCALIYEEIDILTAEGPRIQQGAKTSLOEEMDSFSGSQKVE |
| | | PITEPEVESKYLISTEEVSYFNVQSRVKYLDATPVTKGVASAVVSDEKQDESLKPSEEKE |
| | | ESSSESGTEEVATVKIQEAEGGLIKEDGPMIHTPLVDTVSEEGDIVHLTTSITNAKEVNW |
| | | YFENKLVPSDEKFKCLQDQNTYTLVIDKVNTEDHQGEYVCEALNDSGKTATSAKLTVVKR |
| | | AAPVIKRKIEPLEVALGHLAKFTCEIQSAPNVRFQWFKAGREIYESDKCSIRSSKYISSL |
| | | EILRTQVVDCGEYTCKASNEYGSVSCTATLTVTEAYPPTFLSRPKSLTTFVGKAAKFICT |
| | | VTGTPVIETIWQKDGAALSPSPNWKISDAENKHILELSNLTIQDRGVYSCKASNKFGADI |
| | | CQAELIIIDKPHFIKELTPVQSAINKKVHLECQVVEDRKVTVTWSKDGQKLPPGKDYKIC |
| | | FEDKIATLEIPLAKLKDSGTYVCTASNEAGSSSCSATVTVREPPSFVKKVDPSYLMLPGE |
| | | SARLHCKLKGSPVIQVTWFKNNKELSESNTVRMYFVNSEAILDITDVKVEDSGSYSCEAV |
| | | NDVGSDSCSTEIVIKEPPSFIKTLEPADIVRGTNALLQCEVSGTGPFEISWFKDKKQIRS |
| | | SKKYRLFSQKSLVCLEIFSFNSADVGEYECVVANEVGKCGCMATHLLKEPPTFVKKVDDL |
| | | IALGGQTVTLQAAVRGSEPISVTWMKGQEVIREDGKIKMSFSNGVAVLLIPDVQISFGGK |
| | | YTCLAENEAGSQTSVGELIVKEPAKIIERAELIQVTAGDPATLEYTVAGTPELKPKWYKD |
| | | GRPLVASKKYRISFKNNVAQLKFYSAEUIDSGQYTFEISNEVGSSSCETTFTVLDRDIAP |
| | | FFTKPLRNVDSVVNGTCRLDCKIAGSLPMRVSWFKDGKEIAASDRYRIAFVEGTASLEII |
| | | RVDMNDAGNFTCRATNSVGSKDSSGALIVQEPPSFVTKPGSKDVLPGSAVCLKSTFQGST |
| | | PLTIRWFKGNKELVSGGSCYITKEALESSLELYLVKTSDSGTYTCKVSNVAGGVECSANL |
| | | FVKEPATFVEKLEPSQLLKKGDATQLACKVTGTPPIKITWFANDREIKESSKHRMSFVES |
| | | TAVLRLTDVGIEDSGEYMCEAQNEAGSDHCSSIVIVKESPYFTKEFKPIEVLKEYDVMLL |
| | | AEVAGTPPFEITWFKDNTILRSGRKYKTFIQDHLVSLQILKFVAADAGEYQCRVTNEVGS |
| | | SICSARVTLREPPSFIKKIESTSSLRGGTAAFQATLKGSLPITVTWLKDSDEITEDDNIR |
| | | MTFENNVASLYLSGIEVKHDGKYVCQAKNDAGIQRCSALLSVKEPATITEEAVSIDVTQG |
| | | DPATLQVKFSGTKEITAKWFKDGQELTLGSKYKISVTDTVSILKIISTEKKDSGEYTFEV |
| | | QNDVGRSSCKARINVLDLIIPPSFTKKLKKMDSIKGSFIDLECIVAGSHPISIQWFKDDQ |
| | | EISASEKYKFSFHDNTAFLEISQLEGTDSGTYTCSATNKAGHNQCSGHLTVKEPPYFVEK |
| | | PQSQDVNPNTRVQLKALVGGTAPMTIKWFKDNKELHSGAARSVWKDDTSTSLELFAAKAT |
| | | VDSGTYICQLSNDVGTATSKATLFVKEPPQFIKKPSPVLVLRNGQSTTFECQITGPKIRV |
| | | SWYLDGNEITAIQKHGISFIDGLATFQISGARVENSGTYVCEARNDAGTASCSIELKVKE |
| | | PPTFIRELKPVEVVKYSDVELECEVTGTPPFEVVVLKNNREIRSSKKYTLTDRVSVFNLH |
| | | TTKCDPSDTGEYQCIVSNEGGSCSCSTRVALKEPPSFIKKIENTTTVLKSSATFQSTVAG |
| | | SPPISITWLKDDQILDEDDNVYISFVDSVATLQIRSVDNGHSGRYTCQAKNESGVERCYA |
| | | FLLVQEPAQIVEKAKSVDVTEKDPMTLECVVAGTPELKVKWLKDGKQIVPSRYFSMSFEN |
| | | NVASFRIQSVMKQDSGQYTFKVENDFGSSSCDAYLRVLDQNIPPSFTKKLTKMDKVLGSS |
| | | IHMECKVSGSLPISAQWFKDGKEISTSAKYRLVCHERSVSLEVNNLELEDTANYTCKVSN |
| | | VAGDDACSGILTVKEPPSFLVKPGRQQAIPDSTVEFKAILKGTPPFKIKWFKDDVELVSG |
| | | PKCFIGLEGSTSFLNLYSVDASKTGQYTCHVTNDVGSDSCTTMLLVTEPPKFVKKLEASK |
| | | IVKAGDSSRLECKIAGSPEIRVVWFRNEHELPASFKYRMTFIDSVAVIQMNNLSTEDSGD |
| | | FICEAQNPAGSTSCSTKVIVKEPPVFSSFPTIVETLKNAEVSLECELSGTPPFEVVWYKD |
| | | KRQLRSSKKYKIASKNFHTSIHILNVDTSDIGEYHCKAQNEVGSDTCVCTVKLKEPPRFV |
| | | SKLNSLTVVAGEPAELQASIEGAQPIFVQWLKEKEEVIRESENMRITFVENVATLQFAKA |
| | | EPANAGKYICQIKNDGGMEENMATLMVLEPAVIVEKAGPMTVTVGETCTLECKVAGTPEL |
| | | SVEWYKDGKLLTSSQKHKFSFYNKISSLRILSVERQDAGTYTFQVQNNVGKSSCTAVVDV |
| | | SDRAVPPSFTRRLKNTGGVLGASCILECKVAGSSPISVAWFHEKTKIVSGAKYQTTFSDN |
| | | VCTLQLNSLDSSDMGNYTCVAANVAGSDECRAVLTVQEPPSFVKEPEPLEVLPGKNVTFT |
| | | SVIRGTPPFKVNWFRGARELVKGDRCNIYFEDTVAELELFNIDISQSGEYTCVVSNNAGQ |
| | | ASCTTTILFVKEPAAFLKRLSDHSVEPGKSHLESTYTGTLPISVTWKKDGFNITTSEKCN |
| | | IVTTEKTCILEILNSTKRDAGQYSCEIENEAGRDVCGALVSTLEPPYFVTELEPLEAAVG |
| | | DSVSLQCQVAGTPEITVSWYKGDTKLRPTPEYRTYFTNNVATLVFNKVNINDSGEYTCKA |
| | | ENSIGTASSKTVFRIQERQLPPSFARQLKDIEQTVGLPVTLTCRLNGSAPIQVCWYRDGV |
| | | LLRDDENLQTSFVDNVATLKILQTDLSHSGQYSCSASNPLGTASSSARLTAREPKKSPFF |
| | | DIKPVSIDVTAGESADFECHVTGAQPMRITWSKDNKEIRPGGNYTITCVGNTPHLRILKV |
| | | GKGDSGQYTCQATNDVGKDMCSAQUSVKEPPKFVKKLEASKVAKQGESIQLECKISGSPE |
| | | IKVSWFRNDSELHESWKYNMSFINSVALLTrNEASAEDSGDYICEAHKGVGDASCSTALT |
| | | VKAPPVFTQKPSPVGALKGSDVILQCEISGTPPFEVVWVKDRKQVRNSKKFKITSKHFDT |
| | | SLHILNLEASDVGEYHCKATNEVGSDTCSCSVKFKEPPRFVKKLSDTSTLIGDAVELRAI |
| | | VEGFQPISVVWLKDRGEVIRESENTRISFIDNIATLQLGSPEASNSGKYICQIKNDAGMR |
| | | ECSAVLTVLEPARIIEKPEPMTVTTGNPFALECVVTGTPELSAKWFKDGRELSADSKHHI |
| | | TFINKVASLKIPCAEMSDKGLYSFEVKNSVGKSNCTVSVHVSDRIVPPSFIRKLKDVNAI |
| | | LGASVVLECRVSGSAPISVGWFQDGNEIVSGPKCQSSFSENVCTLNLSLLEPSDTGIYTC |
| | | VAANVAGSDECSAVLTVQEPPSFEQTPDSVEVLPGMSLTFTSVIRGTPPFKVKWFKGSRE |
| | | LVPGESCNISLEDFVTELELFEVQPLESGDYSCLVTNDAGSASCTTHLFVKEPATFVKRL |
| | | ADFSVETGSPIVLEATYTGTPPISVSWIKDEYLISQSERCSITMTEKSTILEILESTIED |
| | | YAQYSCLIENEAGQDICEALVSVLEPPYFIEPLEHVEAVIGEPATLQCKVDGTPEIRISW |
| | | YKEHTKLRSAPAYKMQFKNNVASLVINKVDHSDVGEYSCKADNSVGAVASSAVLVIKERK |
| | | LPPFFARKLKDVHETLGFPVAFECRINGSEPLQVSWYKDGVLLKDDANLQTSFVHNVATL |
| | | QILQTDQSHIGQYNCSASNPLGTASSSAKLILSEHEVPPFFDLKPVSVDLALGESGTFKC |
| | | HVTGTAPIKITWAKDNREIRPGGNYKMTLVENTATLTVLKVGKGDAGQYTCYASNIAGKD |
| | | SCSAHLGVQEPPRFIKKLEPSRIVKQDEFTRYECKIGGSPEIKVLWYKDETEIQESSKFR |
| | | MSFVDSVAVLEMHNLSVEDSGDYTCEAHNAAGSASSSTSLKVKEPPIFRKKPHPIETLKG |
| | | ADVHLECELQGTPPFHVSWYKDKRELRSGKKYKIMSENFLTSIHILNVDAADIGEYQCKA |
| | | TNDVGSDTCVGSIALKAPPRFVKKLSDISTVVGKEVQLQTTIEGAEPISVVWFKDKGEIV |
| | | RESDNIWISYSENIATLQFSRVEPANAGKYTCQIKNDAGMQECFATLSVLEPATIVEKPE |
| | | SIKVTTGDTCTLECTVAGTPELSTKWFKDGKELTSDNKYKISFFNKVSGLKIINVAPSDS |
| | | GVYSFEVQNPVGKDSCTASLQVSDRTVPPSFTRKLKETNGLSGSSVVMECKVYGSPPISV |
| | | SWFHEGNEISSGRKYQTTLTDNTCALTVNMLEESDSGDYTCIATNMAGSDECSAPLTVRE |
| | | PPSFVQKPDPMDVLTGTNVTFTSIVKGTPPFSVSWFKGSSELVPGDRCNVSLEDSVAELE |
| | | LFDVDTSQSGEYTCIVSNEAGKASCTTHLYJKAPAKFVKRLNDYSIEKGKPLILEGTFTG |
| | | TPPISVTWKKNGINVTPSQRCNITTTEKSAILEIPSSTVEDAGQYNCYIENASGKDSCSA |
| | | QILILEPPYFVKQLEPVKVSVGDSASLQCQLAGTPOIGVSWYKGDTKLRPTTTYKMHFRN |
| | | NVATLVFNQVDINDSGEYICKAENSVGEVSASTFLTVQEQKLPPSFSRQLRDVQETVGLP |
| | | VVFITAISGSEPISVSWYKDGKPLKDSPNVQTSFLDNTATLNIFKTDRSLAGQYSCTATN |
| | | PIGSASSSARLILTEGKNPPFFUIRLAPVDAVVGESADFECHVTGTQPIKVSWAKDSREI |
| | | RSGGKYGISYLENSAHLTVLKVDKGDSGQYTCYAVNEVGKDSCTAQLNIKERLIPPSFTK |
| | | RLSETVEETEGNSFKLEGRVAGSQPITVAWYKNNIEIQPTSNCEITFKNNTLVLQVRKAG |
| | | MNDAGLYTCKVSNDAGSALCTSSIVIKEPKKPPVFDQHLTPVTVSEGEYVQLSCHVQGSE |
| | | PIRIQWLKAGREIKPSDRCSFSFASGTAVLELRDVAKADSGDYVCKASNVAGSDTTKSKV |
| | | TIKDKPAVAPATKKAAVDGRLFFVSEPQSIRWEKTTATFIAKVGGDPTPNVKWTICGKWR |
| | | QLNQGGRVFIHQKGDEAKLEIRDTTKTDSGLYRCVAFNEHGEIESNVNLQVDERKKQEKI |
| | | EGDLRAMLKKTPILKKGAGEEEEIDIMELLKNVDPKEYEKYARMYGITDFRGLLQAFELL |
| | | KQSQEEETHRLEIEEIERSERDEKEFEELVSFIQQRLSQTEPVTLIKDIENQTVLKDNDA |
| | | VFEIDIKINYPEIKLSWYKGTEKLEPSDKFEISIDGDRHTLRVKNCQLKDQGNYRLVCGP |
| | | HIASAKLTVIEPAWERHLQDVTLKEGQTCTMTCQFSVPNVKSEWFRNGRILKPQGRHKTE |
| | | VEHKVHKLTIADVRAEDQGQYTCKYEDLETSAELRIEAEPIQFTKRIQNIVVSEHQSATF |
| | | ECEVSFDDAIVTWYKGPTELTESQKYNFRNDGRCHYMTIHNVTPDDEGVYSVIARLEPRG |
| | | EARSTAELYLTTKEIKLELKPPDIPDSRVPIPTMPIRAVPPEEIPPVVAPPIPLLLPTPE |
| | | EKKPPPKRIEVTKKAVKKDAKKVVAKPKEMTPREEIVKKPPPPTTLIPAKAPEIIDVSSK |
| | | AEEVKIMTITRKKEVQKEKEAVYEKKQAVHKEKRVFIESFEEPYDELEVEPYTEPFEQPY |
| | | YEEPDEDYEEIKVEAKKEVHEEWEEDFEEGQEYYEREEGYDEGEEEWEEAYQEREVIQVQ |
| | | KEVYEESHERKVPAKVPEKKAPPPPKVIKKPVIEKIEKTSRRMEEEKVQVTKVPEVSKKI |
| | | VPQKPSRTPVQEEVIEVKVPAVHTKKMVISEEKMFFASHTEEEVSVTVPEVQKEIVTEEK |
| | | IHVAVSKRVEPPPKVPELPEKPAPEEVAPVPIPKKVEPPAPKVPEVPKKPVPEEKKPVPV |
| | | PKKEPAAPPKVPEVPKKPVPEEKIPVPVAKKKEAPPAKVPEVQKRVVTEEKITIVTQREE |
| | | SPPPAVPEIPKKKVPEERKPVPRKEEEVPPPPKVPALPKKPVPEEKVAVPVPVAKKAPPP |
| | | RAEVSKKTWEEKRFVAEEKLSFAVPQRVEVTRHEVSAEEEVVSYSEEEEGVSISVYREEE |
| | | REEEEEAEVTEYEVMEEPEEYVVEEKLHIISKRVEAEPAEVTERQEKKIVLKPKIPAKIE |
| | | EPPPAKVPEAPKKIVPEKKVPAPVPKKEKVPPPKVPEEPKKPVPEKKVPPKVIKMEEPLP |
| | | AKVTERHMQITQEEKVLVAVTKKEAPPKARVPEEPKRAVPEEKVLKLKPKREEEPPAKVT |
| | | EFRKRVVKEEKVSIEAPKREPQPIKEVTtMEEKERAYTLEEEAVSVQREEEYEEYEEYDY |
| | | KEFEEYEPTEEYDQYEEYEEREYERYEEHEEYITEPEKPIPVKPVPEEPVPTKPKAPPAK |
| | | VLKKAVPEEKVPVPIPKKLKPPPPKVPEEPKKVFEEKIRISITKREKEQVTEPAAKVPMK |
| | | PKRVVAEEKVPVPRKEVAPPVRVPEVPKELEPEEVAFEEEVVTHVEEYLVEEEEEYIHEE |
| | | EEFITEEEVVPVTPVKVPEVPRKPVPEEKKPVPVPKKXEAPPAKVPEVPKKPEEKVPVLI |
| | | PKKEKPPPAKVPEVPKKPVPEEKVPVPVPKKVEAPPAKVPEVPKKPVPEKKVPVPAPKKV |
| | | EAPPAKVPEVPKKLIPEEKKPTPVPKKVEAPPPKVPKKREPVPVPVALPQEEEVLFEEEI |
| | | VPEEEVLPEEEEVLPEEEEVLPEEEEVLPEEEEIPPEEEEVPPEEEYVPEEEEFVPEEEV |
| | | LPEVKPKVPVPAPVPEIKKKVTEKKVVIPKKEEAPPAKVPEVPKKVEEKRIILPKEEEVL |
| | | PVEVTEEPEEEPISEEEIPEEPPSIEEVEEVAPPRVPEVIKKAVPEAPTPVPKKVEAPPA |
| | | KVSKKIPEEKVPVPVQKKEAPPAKVPEVPKKVPEKKVLVPKKEAVPPAKGRTVLEEKVSV |
| | | AFRQEVVVKERLELEVVEAEVEEIPEEEEFHEVEEYFEEGEFHEVEEFIKLEQHRVEEEH |
| | | RVEKVHRVIEVFEAEEVEVFEKPKAPPKGPEISEKIIPPKKPPTKVVPRKEPPAKVPEVP |
| | | KKIVVEEKVRVPEEPRVPPTKVPDVLPPKEVVPEKKVPVPPAKKPEAPPPKVPEAPKEVV |
| | | PEKKVPVPPPKKPEVPPTKVPEVPKAAVPEKKVPEAIPPKPESPPPEVPEAPKEVVPEKK |
| | | VPAAPPKKPEVTPVKVPEAPKEVVPEKKVPVPPPKKPEVPPTKVPEVPKVAVPEKKVPEA |
| | | IPPKPESPPPEVFEEPEEVALEEPPAEVVEEPEPAAPPQVTVPPKKPVPEKKAPAVVAKK |
| | | PELPPVKVPEVPKEVVPEKKVPLVVPKKPEAPPAKVPEVPKEVVPEKKVAVPKKPEVPPA |
| | | KVPEVPKKPVLEEKPAVPVPERAESPPPEVYEEPEETAPEEEIAPEEEKPVPVAEEEEPE |
| | | VPPPAVPEEPKKIIPEKKVPVIKKPEAPPPKEPEPEKVIEKPKLKPRPPPPPPAPPKEDV |
| | | KEKIFQLKAIPKKKVPEKPQVPEKVELTPLKVPGGEKKVRKLLPERKPEPKEEVVLKSVL |
| | | RKRPEEEEPKVEPKKLEKVKKPAVPEPPPPKPVEEVEVPTVTKRERKIPEPTKVPEIKPA |
| | | IPLPAPEPKPKPEAEVKTIKPPPVEPEPTPIAAPVTVPVVGKXAEAKAPKEEAAKPKGPI |
| | | KGVPKKTPSPIEAERRKLRPGSGGEKPPDEAPFTYQLKAVPLKFVKEIKDIILTESETVG |
| | | SSAIFECLVSPSTAITTWMKDGSNIRESPKHRFIADGKDRKLHIIDVQLSDAGEYTCVLR |
| | | LGNKEKTSTAKLVVEELPVRFVKTLEEEVTVVKGQPLYLSCELNKERDVVWRKDGKIVVE |
| | | KPGRIVPGVIGLMRALTINDADDTDAGTYTVTVENANNLECSSCVKVVEVIRDWLVKPIR |
| | | DQHVKPKGTAIFACDIAKDTPNIKWFKGYDEIPAEPNDKTEILRDGNHLYLKIKNAMPED |
| | | IAEYAVEIEGKRYPAKLTLGEREVELLKPIEDVTIYEKESASFDAEISEADIPGQWKLKG |
| | | ELLRPSPTCEIKAEGGKRFLTLRKVKLDQAGEVLYQALNAITTAILTVKEIELDFAVPLK |
| | | DVTVPERRQARFECVLTREANVIVVSKGPDUKSSDKFDIIADGKKHILVINDSQFDDEGV |
| | | YTAEVEGKKTSARLFVTGIRLKFMSPLEDQTVKEGETATFVCELSHFKMHVVWFKNDAKL |
| | | HTSRTVLISSEGKTHKLEMKEVTLDDISQIKAQVKELSSTAQLKVLEADPYFTVKLHDKT |
| | | AVEKDEITLKCEVSKDVPVKWFKDGEEIVPSPKYSIKADGLRRILKIKKADLKDKGEYVC |
| | | DCGTDKTXANVTVEARLIKVEKPLYGVEVFVGETAHFEIELSEPDVHGQWKLKGQPLTAS |
| | | PDCEIIEDGKKHILILHNCQLGMTGEVSFQAANAKSAANLKVKELPLIFTTPLSDVKVFE |
| | | KDEAKFECEVSREPKTFRWLKGTQEITGDDRFELIKDGTKHSMVIKSAAFEDEAKYMFEA |
| | | EDKHTSGKLUEGIRLKFLTPLKDVTAKEKESAVFTVELSHDNIRVKWFECNDQRLHTTRS |
| | | VSMQDEGKTHSITFKDLSIDDTSQIRVEAMGMSSEAKLTVLEGDPYFTGKLQDYTGVEKD |
| | | EVILQCEISKADAPVKWFKDGKEIKPSKNAVIKADGKKRMLILKKALKSDIGQYTCDCGT |
| | | DKTSGKLDIEDREIKLVRPLHSVEVMETETARFETEISEDDIHANWKLKGEALLQTPDCE |
| | | IKEEGKIHSLVLHNCRLDQTGGVDFQAANVKSSAHLRVKPRVIGLLRPLKDVTVTAGETA |
| | | TFDCELSYEDIPVEWYLKGKKLEPSDKVVPRSEGKVHTLTLRDVKLEDAGEVQLTAKDFK |
| | | THANLFVKEPPVEFTKPLEDQTVEEGATAVLECEVSRENAKVKWFKNGTEILKSKKYEIV |
| | | ADGRVRKLVIHDCTPEDIKTYTCDAKDFKTSCNLNVVPPHVEFLRPLTDLQVREKEMARF |
| | | ECELSRENAKVKWFKDQAEIKKGKKYDIISKGAVRILVINKCLLDDEAEYSCEVRTARTS |
| | | GMLTVLEEEAVFTKNLANIEVSETDTIKLVCEVSKPGAEVIVVYKGDEEHETGRYEILTE |
| | | GRKRILVIQNAHLEDAGNYNCRLPSSRTDGKVKVHELAAEFISKPQNLEILEGEKAEFVC |
| | | SISKESFPVQWKRDDKTLESGDKYDVIADGKKRVLVVKDATLQDMGTYVVMVGAARAAAH |
| | | LTVIEKLRIVVPLKDTRVKEQQEVVFNCEVNTEGAKAKWFRNEEAIFDSSKYIILQKDLV |
| | | YTLRIRDAHLDDQANYNVSLTNHRGENVKSAANLIVEEEDLRIVEPLKDIETMEKKSVTF |
| | | WCKVNRLNVTLKWTKNGEEVPFDNRVSYRVDKYKHMLTIKDCGFPDEGEYIVTAGQDKSV |
| | | AELLIIEAPTEFVEHLEDQTVTEFDDAVFSCQLSREKANVKWYRNGREIKEGKKYKFEKD |
| | | GSIHRLIIKDCRLDDECEYACGVEDRKSRARLFVEEIIPVEHRPPQDILEAPGADVVFLA |
| | | ELNKDKVEVQWLRNNMVVVQGDKHQMMSEGKIHRLQICDIKPRD0GEYRFIAKDKEARAK |
| | | LELAAAPKIKTADQDLVVDVGKPLTMVVPYDAYPKAEAEWFKENEPLSTKTIDTTAEQTS |
| | | FRILEAKKGDKGRYKIVLQNKHGKAEGFINLKVIDVPGPVRNLEVTETFDGEVSLAWEEP |
| | | LTDGGSKIIGYVVERRDIKRKTWVLATDRAESCEFTVTGLQKGGVEYLFRVSARNRVGTG |
| | | EPVETDNPVEARSKYDVPGPPLNVTITDVNRFGVSLTWEPPEYDGGAEITNYVIELRDKT |
| | | SIRWDTAMTVRAEDLSATVTDVVEGQEYSFRVRAQNRIGVGKPSAATPFVKVADPIERPS |
| | | PPVNLTSSDQTQSSVQLKWEPPLKDGGSPILGYIIERCEEGKDNWIRCNMKLVPELTYKV |
| | | TGLEKGNKYLYRVSAENKAGVS0PSEILGPLTADDAFVEPTMDLSAFKDGLEVIVPNPIT |
| | | ILWSTGYPRPTATVVCFGDKVLETGDRVKMKTLSAYAELVISPSERSDKGIYTLKLENRV |
| | | KTISGEIDVNVIARPSAPKELKFGDITKDSVHLTWEPPDDDGGSPLTGYVVEKREVSRKT |
| | | WTKVMDFVTDLEFTVPDLVOGKEYLFKVCARNKCGPGEPAYVDEPVNMSTPATVPDPPEN |
| | | VKWRDRTANSIFLTWDPPKNDGGSRIKGYIVERCPRGSDKWVACGEPVAETKMEVTGLEE |
| | | GKWYAYRVKALNRQGASKPSRPTEEIQAVDTQEAPEIFLDVKLLAGLTVKAGTKIELPAT |
| | | VTGKPEPKITWTKADMILKQDKRITIENVPKKSTVTIVDSKRSDTGTYIIEAVNVCGRAT |
| | | AVVEVNVLDKPGPPAAFDITDVTNESCLLTWNPPRDDGGSKITNYVVERRATDSEVWHKL |
| | | SSTVKDTNFKATKLIPNKEYIFRVAAENMYGVGEPVQASPITAKYQFDPPGPPTRLEPSD |
| | | ITKDAVTLTWCEPDDDGGSPITGYWVERLDPDTDKWVRCNKMPVKDTTYRVKGLTNKKKY |
| | | RFRVLAENLAGPGKPSKSTEPILIKDPIDPPWPPCKPTVKDVGKTSVRLNWTKPEHDGGA |
| | | KIESYVIEMLKTGTDEWVRVAEGVPTTQHLLPGLMEGOEYSFRVRAVNKAGESEPSEPSD |
| | | PVLCREKLYPPSPPRWLEVINITKNTADLKWTVPEKDGGSPITNYIVEKRDVRRKGWQTV |
| | | DTTVKDTKCIVTPLTEGSLYVFRVAAENAIGQSDYTEIEDSVLAKDTPTTPGPPYALAVV |
| | | DVTKRHVDLKWEPPKNDGGRPIQRYVIEKKERLGTRWVKAGKTAGPDCNFRVTDVIEGTE |
| | | VQFQVRAENEAGVGHPSEPTEILSIEDPTSPPSPPLDLHVTDAGRKHIAIAWKPPEKNGG |
| | | SPHGYHVEMCPVGTEKVVMRVNSRPIKDLKFKVEEGVVPDKEYVLRVRAVNAIGVSEPSE |
| | | ISENVVAKDPDCKPTIDLETHDHVIEGEKLSIPVPFRAVPVPTVSVVHKDGKEVKASDRL |
| | | TMKNDHISAHLEVPKSVRADAGIYTITLENKLGSATASINVKVIGLPGPCKDIKASDITK |
| | | SSCKLTWEPPEFDGGTPILHYVLERREAGRRTYIPVMSGENKLSWTVKDLIPNGEYFFRV |
| | | KAVNKVGGGEYIELKNPVIAQDPKQPPDPPVDVEVHNPTAEAMTITWKPPLYDGGSKIMG |
| | | YIIEKIAKGEERWKRCNEHLVPILTYTAKGLEEGKEYQFRVRAENAAGISEPSRATPPTK |
| | | AVDPIDAPKVILRTSLEVKRGDEJALDASISGSPYPTITWIKDENVIVPEEIKKRAAPLV |
| | | RRRKGEVQEEEPFVLPLTQRLSIDNSKKGESQLRVRDSLRPDHGLYMIKVENDHGIAKAP |
| | | CTVSVLDTPGPPINFVFEDIRKTSVLCKWEPPLDDGGSEIINYTLEKKDKTKPDSEWIVV |
| | | TSTLRHCKYSVTKLIEGKEYLFRVRAENRFGPGPPCVSKPLVAKDPFGPPDAPDKPIVED |
| | | VTSNSMLVKWNEPKDNGSPILGYWLEKREVNSTHWSRVNKSLLNALKANVDGLLEGLTYV |
| | | FRVCAENAAGPGKFSPPSDPKTAHDPISPPGPPIPRVTDTSSTTIELEWEPPAFNGGGEI |
| | | VGYFVDKQLVGTNEWSRCTEKMIKVRQYTVKEIREGADYKLRVSAVNAAGEGPPGETQPV |
| | | TVAEPQEPPAVELDVSVKGGIQIMAGKTLRIPAVVTGRPVPTKVWTKEEGELDKDRVVID |
| | | NVGTKSELIIKDALRKDHGRYVITATNSCGSKFAAARVEVFDVPGPVLDLKPVVTNRKMC |
| | | LLNWSDPEDDGGSEITGFIIERKDAKMHTWRQPIETERSKCDITGLLEGQEYKFRVIAKN |
| | | KFGCGPPVEIGPILAVDPLGPPTSPERLTYTERTKSTITLDWKEPRSNGGSPIQGYIIEK |
| | | RRHDKPDFERVNKRLCITTSFLVENLDEHQMYEFRVKAVNEIGESEPSLPLNVVIQDDEV |
| | | PPTIKLRLSVRGDTIKVKAGEPVHIPADVTGLPMPKIEWSKNETVIEKPTDALQITKEEV |
| | | SRSEAKTELSIPKAVREDKGTYTVTASNRLGSVFRNVHVEVYDRPSPPRNLAVTDIKAES |
| | | CYLTWDAPLDNGGSEITHYVIDKRDASRKKAEWEEVTNTAVEKRYGIWKLIPNGQYEFRV |
| | | RAVNKYGISDECKSDKVVIQDPYRLPGPPGKPKVLARTKGSMLVSWTPPLDNGGSPITGY |
| | | WLEKREEGSPYWSRVSRAPITKVGLKGVEFNVPRLLEGVKYQFRAMAINAAGIGPPSEPS |
| | | DPEVAGDPIFPPGPPSCPEVKDKTKSSISLGWKPPAKDGGSPIKGYIVEMQEEGTTDWKR |
| | | VNEPDKLITTCECVVPNLKELRKYRFRVKAVNEAGESEPSDTTGEIPATDIQEEPEVFID |
| | | IGAQDCLVCKAGSQIRIPAVIKGRPTPKSSWEFDGKAKKAMKDGVHDIPEDAQLETAENS |
| | | SVIIIPECKRSHTGKYSITAKNKAGQKTANCRVKVMDVPGPPKDLKVSDITRGSCRLSWK |
| | | MPDDDGGDRIKGYVIEKRTIDGKAWTKVNPDCGSTTFVVPDLLSEQQYFFRVRAENRFGI |
| | | GPPVETIQRTTARDPIYPPDPPIKLKIGLITKNTVHLSWKPPKNDGGSPVTHYIVECLAW |
| | | DPTGTKKEAWRQCNKROVEELQFTVEDLVEGGEYEFRVKAVNAAGVSKPSATVGPCDCQR |
| | | PDMPPSIDLKEFMEVEEGTNVNIVAKIKGVPFPTLTWFKAPPKKPDNKEPVLYDTHVNKL |
| | | VVDDTCTLVIPQSRRSDTGLYTITAVNNLGTASKEMRLNVLGRPGPPVGPIKFESVSADQ |
| | | MTLSWFPPKDDGGSKITNYVIEKREANRKTWVHVSSEPKECTTTIPKLLEGHEYVFRIMA |
| | | QNKYGIGEPLDSEPETARNLFSVPGAPDKPTVSSVTRNSMTVNWEEPEYDGGSPVTGYWL |
| | | EMKDTTSKRWKRVNRDPIKAMTLGVSYKVTGLIEGSDYQFRVYAINAAGVGPASLPSDPA |
| | | TARDPIAPPGPPFPKVTDWTKSSADLEWSPPLKDGGSKVTGYIVEYKEEGKEEWEKGKDK |
| | | EVRGTKLVVTGLKEGAFYKFRVSAVNIAGIGEPGEVTDVIEMKDRLVSPDLQLDASVRDR |
| | | IVVHAGGVIRIIAYVSGKPPPTVTWNMNERTLPQEATIETTAISSSMVIKKCQRSHQGVY |
| | | SLLAKNEAGERKKTHVDVLDVPGPVGTPFLAHNLTNESCKLTVVFSPEDDGGSPITNYVI |
| | | EKRESDRRAWTPVTYTVTRQNATVQGLIQGKAYFFRIAAENSIGMGPFVETSEALVIREP |
| | | ITVPERPEDLEVKEVTKNTVTLTWNPPKYDGGSEIINYVLESRLIGTEKFHKVTNDNLLS |
| | | RKYTVKGLKEGDTYEYRVSAVKIVGQGKPSFCTKPITCKDELAPPTLHLDFRDKLTIRVG |
| | | EAFALTGRYSGKPKPKVSWFKDEADVLEDDRTHIKTTPATLALEKIKAKRSDSGKYCVVV |
| | | ENSTGSRKGFCQVNVVDRPGPPVGPVSFDEVTKDYMVISWKPPLDDGGSKITNYIIEKKE |
| | | VGKDVWMPVTSASAKTTCKVSKLLEGKDYIFRIHAENLYGISDPLVSDSMKAKDRFRVPD |
| | | APDQPIVTEVTKDSALVTWNKPHDGGKPITNYILEKRETMSKRWARVTKDPIHPYTKFRV |
| | | PDLLEGCQYEFRVSAENEIGIGDPSPPSKPVFAKDPIAKPSPPVNPEAIDTTCNSVDLTW |
| | | QPPRHDGGSKILGYIVEYQKVGDEEWRRANHTPESCPETKYKVTGLRDGQTYKFRVLAVN |
| | | AAGESDPAHVPEPVLVKDRLEPPELILDANMAREQHIKVGDTLRLSAIIKGVPFPKVTWK |
| | | KEDRDAPTKARIDVTPVGSKLEIRNAAHEDGGIYSLTVENPAGSKTVSVKVLVLDKPGPP |
| | | RDLEVSEIRKDSCYLTWKEPLDDGGSVITNYVVERRDVASAQWSPLSATSKKKSHFAKHL |
| | | NEGNQYLFRVAAENQYGRGPFVETPKPIKALDPLHPPGPPKDLHHVDVDKTEVSLVWNKP |
| | | DRDGGSPITGYLVEYQEEGTQDWIKFKTVTNLECVVTGLQQGKTYRFRVKAENIVGLGLP |
| | | DTTIPIECQEKLVPPSVELDVKLIEGLVVKAGTTVRFPAIIRGVPVPTAKWTTDGSEIKT |
| | | DEHYTVETONFSSVLTIKNCLRRDTGEYQITVSNAAGSKIVAVHLTVLDVPGPPTGPINI |
| | | LDVTPEHMTISWQPPKDDGGSPVINYIVEKQDTRKDTWGVVSSGSSKTKLKIPHLQKGCE |
| | | YVFRVRAENKIGVGPPLDSTPTVAKHKFSPPSPPGKPVVTDITENAATVSWTLPKSDGGS |
| | | PITGYYMERREVTGKWVRVNKTPIADLKFRVTGLYEGNTYEFRVFAENLAGLSKPSPSSD |
| | | PIKACRPIKPPGPPINPKLKDKSRETADLVWTKPLSDGGSPILGYWECQKPGTAQVVNRI |
| | | NKDELIRQCAFRVPGLIEGNEYRFRIKAANIVGEGEPRELAESVIAKDILHPPEVELDVT |
| | | CRDVITVRVGQTIRILARVKGRPEPDITWTKEGKVLVREKRVDLIQDLPRVELQIKEAVR |
| | | ADHGKYIISAKNSSGHAQGSAIVNVLDRPGPCQNLKVTNVTKENCTISWENPLDNGGSEI |
| | | TNFIVEYRKPNQKGWSIVASDVTKRLIKANLLANNEYYFRVCAENKVGVGPTIETKTPIL |
| | | AINPIDRPGEPENLHIADKGKTFVYLKWRRPDYDGGSPNLSYHVERRLKGSDDWERVHKG |
| | | SIKETHYMVDRCVENQIYEFRVQTKNEGGESDWVKTEEVVVKEDLQKPVLDLKLSGVLTV |
| | | KAQDTIRLEAGVRGKPFPEVAWTKDKDATDLTRSPRVKIDTRADSSKFSLTKAKRSDGGK |
| | | YVVTATNTAGSFVAYATVNVLDKPGPVRNLKIVDVSSDRCTVCWDPPEDDGGCEIQNYIL |
| | | EKCETKRMVWSTYSATVLTPGTTVTRLIEGNEYIFRVRAENKIGTGPPTESKPVIAKTKY |
| | | DKPGRPDPPEVTKVSKEEMTVVWNPPEYDGGKSITGYFLEKKEKHSTRWVPVNKSAIPER |
| | | RMKVQNLLPDHEYQFRVKAENEIGIGEPSLPSRPVVAKDPIEPPGPPTNFRVVDITKHSI |
| | | TLGWGKPVYDGGAPHGYVVEMRPKIADASPDEGVVKRCNAAAQLVRKEFTVTSLDENQEY |
| | | EFRVCAQNQVGIGRPAELKEAIKPKEILEPPEIDLDASMRKLVIVRAGCPIRLFAIVRGR |
| | | PAPKVTWRKVGIDNVVRKGQVDLVDTMAFLVIPNSTRDDSGKYSLTLVNPAGEKAVFVNV |
| | | RVLDTPGPVSDLKVSDVTKTSGHVSWAPPENDGGSQVTHYIVEKREADRKTWSTVTPEVK |
| | | KTSFHVTNLVPGNEYYFRVTAVNEYGPGVPTDVPKPVLASDPLSEPDPPRKLEVTEMTKN |
| | | SATLAWLPPLRDGGAKIDGYITSYREEEQPAORWTEYSVVKDLSLVVTGLKEGKKYKFRV |
| | | AARNAVGVSLPREAEGVYEAKEQLLPPKILMPEQITIKAGKKLRIEAHVYGKPHPTCKWK |
| | | KGEDEVVTSSHLAVHKADSSSILIIKDVTRKDSGYYSLTAENSSGTDTQKIKVVVMDAPG |
| | | PPQPPFDISDIDADACSLSWHIPLEDGGSNITNYIVEKCDVSRGDWVTALASVTKTSCRV |
| | | GKLIPGQEYIFRVRAENRFGISEPLTSPKMVAQFPFGVPSEPKNARVTKVNKDCIFVAWD |
| | | RPDSDGGSPIIGYLIERKERNSLLWVKANDTLVRSTEYPCAGLVEGLEYSFRIYALNKAG |
| | | SSPPSKPTEYVTARMPVDPPGKPEVIDVTKSTVSLIWARPKHDGGSKIIGYFVEACKLPG |
| | | DKWVRCNTAPHQIPQEEYTATGLEEKAQYQFRAIARTAVNISPPSEPSDPVTILAENVPP |
| | | RIDLSVAMKSLLTVKAGTNVCLDATVFGKPMPTVSWKKDGTLLKPAEQIKMAMQRNLCTL |
| | | ELFSVNRKDSGDYTITAENSSGSKSATIKLKVLDKPGPPASVKINKMYSDRAMLSWEPPL |
| | | EDGGSEITNYIVDKRETSRPNWAQVSATVPITSCSVEKLIEGHEYQFRICAENKYGVGDP |
| | | VFTEPAIAKNPYDPPGRCDPPVISNITKDHMTVSWKPPADDGGSPITGYLLEKRETQAVN |
| | | WTKVNRKPIIERTLKATGLQEGTEYEFRVTAINKAGPGKPSDASKAAYARDPQYPPAPPA |
| | | FPKVYDTTRSSVSLSWGKPAYDGGSPIIGYLVEVKRADSDNWVRCNLPQNLQKTRFEVTG |
| | | LMEDTQYQFRVYAVNKIGYSDPSDVPDKHYPKDILIPPEGELDADLRKTLILRAGVTMRL |
| | | YVPVKGRPPPKITWSKPNVNLRDRIGLDIKSTDFDTFLRCENVNKYDAGKYILTLENSCG |
| | | KKEYTIVVKVLDTPGPPVNVTVKEISKDSAYVTWEPPIIDGGSPIINYVV0KRDAERKSW |
| | | STVTTECSKTSFRVANLEEGKSYFFRVFAENEYGIGDPGETRDAVKASQTPGPVVDLKVR |
| | | SVSKSSCSIOWKKPHSDGGSRIIGYVVDFLTEENKWQRVMKSLSLQYSAKDLTEGKEYTF |
| | | RVSAENENGEGTPSEITVVARDDVVAPDLDLKGLPDLCYLAKENSNFRLKIPIKGKPAPS |
| | | VSWKKGEDPLATDTRVSVESSAVNTTLIVYDCQKSDAGKYTITLKNVAGTKEGTISIKVV |
| | | GKPGIPTGPIKFDEVTAEAMTLKWAPPKDDGGSEITNYILEKRDSVNNKWVTCASAVQKT |
| | | TFRVTRLHEGMEYTFRVSAENKYGVGEGLKSEPIVARHPFDVPDAPPPPNIVDVRHDSVS |
| | | LTWTDPKKTGGSPITGYHLEFKERNSLLWKRANKTPIRMRDFKVTGLTEGLEYEFRVMAI |
| | | NLAGVGKPSLPSEPVVALDPIDPPGKPEVINITRNSVTLIWTEPKYDGGHKLTGYIVEKR |
| | | DLPSKSWMKANHVNVPHCAFTVTDLVEGGKYEFRIRAKNTAGAISAPSESTETIICKDEY |
| | | EAPTIVLDPTIKDGLTIKAGDTIVLNAISILGKPLPKSSWSKAGKDIRPSDITQITSTPT |
| | | SSMLTIKYATRKDAGEYTITATNPFGTKVEHVKVTVLDVPGPPGPVEISNVSAEKATLTW |
| | | TPPLEDGGSPIKSYILEKRETSRLLWTVVSEDIQSCRHVATKLIQGNEYIFRVSAVNHYG |
| | | KGEPVQSEPVKMVDRFGPPGPPEKPEVSNVTKNTATVSWKRPVDDGGSEITGYHVERREK |
| | | KSLRWVRAIKTPVSDLRCKVTGLQEGSTYEFRVSAENRAGIGPPSEASDSVLMKDAAYPP |
| | | GPPSNPHVTDTTKKSASLAWGKPHYDGGLEITGYVVEHQKVGDEAWIKDTTGTALRITQF |
| | | VVPDLQTKEKYNFRISAINDAGVGEPAVIPDVEIVEREMAPDFELDAELRRTLVVRAGLS |
| | | IRIFVPIKGRPAPEVTWTKDNINLKNRANIENTESFTLLIIPECNRYDTGKFVMTIENPA |
| | | GKKSGFVNVRVLDTPGPVLNLRPTDITKDSVTLHWDLPLIDGGSRITNYIVHKREATRKS |
| | | YSTATTKCHKCTYKVTGLSEGCEYFFRVMAENTYGIGEPTETTEPVKASEAPSPPDSLNI |
| | | MDITKSTVSLAWPKPKHDGGSKITGYVIEAQRKGSDQWTMITTVKGLECVVRNLTEGEEY |
| | | TFQVMAVNSAGRSAPRESRPVIVKEQTMLPELDLRGIYQKLVIAKAGDNIKVEIPVLGRP |
| | | KPTVTWKKGDQILKQTQRVNFETTATSTILNINECVRSDSGPYPLTARNIVGEVGDVITI |
| | | QWDIPGPPTGPIKFDEVSSDFVTFSVVDPPENDGGVPISNYVVEMRQTDSTTWVELATTV |
| | | IRTTYKATRLTTGLEYQFRVKAQNRYGVGPGITSACIVANYPFKVPGPPGTPQVTAVTKD |
| | | SMTISWHEPLSDGGSPILGYHVERKERNGILWQTVSKALVPGNTFKSSGLTDGIAYEFRV |
| | | IAENMAGKSKPSKPSEPMLALDPIDPPGKPVPLNITRHTVTLKWAKPEYTGGFKITSYIV |
| | | EKRDLPNGRWLKANFSNILENEFTVSGLTEDAAYEFRVIAKNAAGAISPPSEPSDAITCR |
| | | DDVEAPKIKVDVKFKDTVILKAGEAFRLEADVSGRPPPTMEWSKDGKELEGTAKLEIKIA |
| | | DFSTNLVNKDSTRRDSGAYTLTATNPGGFAKHIFNVKVLDRPGPPEGPLAVTEVTSEKCV |
| | | LSWFPPLDDGGAKIDHYIVQKRETSRLAWTNVASEVQVTKIXVTKLLKGNEYIFRVMAVN |
| | | KYGVGEPLESEPVLAVNPYGPPDPPKNPEVTTITKDSMVVCWGHPDSDGGSEIINYIVER |
| | | RDKAGQRWIKCNKKTLTDLRYKVSGLTEGHEYEFRIMAENAAGISAPSPTSPFYKACDTV |
| | | FKPGPPGNPRVLDTSRSSISIAWNKPIYDGGSEITGYMVEIALPEEDEWQIVTPPAGLKA |
| | | TSYTITGLTENQEYKIRIYAMNSEGLGEPALVPGTPKAEDRMLPPEIELDADLRKVVTIR |
| | | ACCTLRLFVPIKGRPAPEVKWARDHGESLDKASIESTSSYTLLIVGNVNRFDSGKYILTV |
| | | ENSSGSKSAFVNVRVLDTPGPPQDLKVKEVTKTSVTLTWDPPLLDGGSKIKNYIVEKRES |
| | | TRKAYSTVATNCHKTSWKVDQLQEGCSYYFRVLAENEYGIGLPAETAESVKASERPLPPG |
| | | KITLMDVTRNSVSLSWEKPEHDGGSRILGYIVEMQTKGSDKWATCATVKVTEATITGLIQ |
| | | GEEYSFRVSAQNEKGISDPRQLSVPVIAKDLVIPPAFKLLFNTFTVLAGEDLKVDVPFIG |
| | | RPTPAVTWHKDNVPLKQTTRVNAESTENNSLLTIKDACREDVGHYVVKLTNSAGEAICTL |
| | | NVIVLDKPGPPTGPVKMDEVTADSITLSWGPPKYDGGSSINNYIVEKRDTSTTTWQIVSA |
| | | TVARTTIKACRLKTGCEYQFRIAAENRYGKSTYLNSEPTVAQYPFKVPGPPGTPVVTLSS |
| | | RDSMEVQWNEPISDGGSRVIGYHLERKERNSILWVKLNKTPIPQTKFKTTGLEEGVEYEF |
| | | RVSAENIVGIGKPSKVSECYVARDPCDPPGRPEAIIVTRNSVTLQWKKPTYDGGSKITGY |
| | | IVEKKELPEGRVVMKASFTNUDTHFEVTGLVEDHRYEFRVIARNAAGVFSEPSESTGAIT |
| | | ARDEVDPPRISMDPKYKDTIVVHAGESFKVDADIYGKPIPTLQWIKGDQELSNTARLEIK |
| | | STDFATSLSVKDAVRVDSGNYILKAKNVAGERSVTVNVKVLDRPGPPEGPVVISGVTAEK |
| | | CTLAWKPPLQDGGSDIINYIVERRETSRLVWTVVDANVQTLSCKVTKLLEGNEYTFRIMA |
| | | VNKYGVGEPLESEPVVAKNPFVVPDAPKAPEVTTVTKDSMIVVWERPASDGGSEILGYVL |
| | | EKRDKEGIRWTRCHKRLIGELRLRVTGLIENHDYEFRVSAENAAGLSEPSPPSAYQKACD |
| | | PIYKPGPPNNPKVIDITRSSVFLSWSKPIYDGGCEIQGYIVEKCDVSVGEWTMCTPPTGI |
| | | NKTNIEVEKLLEKHCYNFRICAINKAGVGEHADVPGPLIVELKLEAPDIDLDLELRKIIN |
| | | IRAGGSLRLFVPIKGRPTPEVKVVGKVDGEIRDAAUDVTSSFTSLVLDNVNRYDSGKYTL |
| | | TLENSSGTKSAFVTVRVLDTPSPPVNLKWIEITKDSVSITWEPPLLDGGSKIKNYIVEKR |
| | | EATRKSYAAVVTNCHKNSWKIDQLQEGCSYYFRVTAENEYGIGLPAQTADPIKVAEVPQP |
| | | PGKITVDDVTRNSVSLSWTKPEHDGGSKHQYIVEMQAKHSEKVVSECARVKSLQAVITNL |
| | | TQGEEYLFRVVAVNEKGRSDPRSLAVPIVAKDLVIEPDVKPAFSSYSVQVGQDLKIEVPI |
| | | SGRPKPTITVVTKDGLPLKQTTRINVTDSLDLTTLSIKETHKDDGGQYGIVVAWVGQKTA |
| | | SIEIVTLDKPDPPKGPVKFDDVSAESITLSWNPPLYTGGCQITNYIVQKRDTTTTVWDVV |
| | | SATVARTTLKVTKLKTGTEYQFRIFAENRYGOSFALESDPIVAQYPYKEPGPPGTPFATA |
| | | ISKDSMVIQWHEPVNNGGSPVIGYHLERKERNSILWTKVNKTIIHDTQFKAQNLEEGIEY |
| | | EFRVYAENIVGVGKASKNSECYVARDPCDPPGTPEPIMVKRNEITLQWTKPVYDGGSMIT |
| | | GYIVEKRDLPDGRWMKASFTNVIETQFTVSGLTEDQRYEFRVIAKNAAGAISKPSDSTGP |
| | | ITAKDEVELPRISMDPKFRDTIVVNAGETFRLEADVHGKPLPTIEWLRGDKEIEESARCE |
| | | IKNTDFKALLIVKDAIRIDGGQYILRASNVAGSKSFPVNVKVLDRPGPPEGPVQVTGVTS |
| | | EKCSLTWSPPLQDGGSDISHYVVEKRETSRLAVTTVVASEWTNSLKVTTLLEGNEYVFRI |
| | | MAVNKYGVGEPLESAPVLMKNPFVLPGPPKSLEVTNIAKDSMTVCWNRPDSDGGSEIIGY |
| | | IVEKRDRSGIRWIKCNKRRITDLRLRVTGLTEDHEYEFRVSAENAAGVGEPSPATVYYKA |
| | | CDPVFKPGPPTNAHIVDTTKNSITLAWGKPIYDGGSEILGYVVEICKADEEEWQIVTPQT |
| | | GLRVTRFEISKLTEHQEYKIRVCALNKVGLGEATSVPGTVKPEDKLEAPELDLDSELRKG |
| | | IVVRAGGSARIHIPFKGRPTPEITWSREEGEFTDKVQIEKGVNYTQLSIDNCDRNDAGKY |
| | | ILKLENSSGSKSAFVTVKVLDTPGPPQNLAVKEVRKDSAFLVWEPPIIDGGAKVKNYVID |
| | | KRESTRKAYANVSSKCSKTSFKVENLTEGAIYYFRVMAENEFGVGVPVETVDAVKAAEPP |
| | | SPPGKVTLTDVSQTSASLMWEKPEHDGGSRVLGYVVEMQPKGTEKWSIVAESKVCNAVVT |
| | | GLSSGQEYQFRVKAYNEKGKSDPRVLGVPVlAKDLTIQPSLKLPFTTYSIQAGEDLKIEI |
| | | PVIGRPRPNISWVKDGEPLKQTTRVNVEETATSTVLHIKEGNKDDFGKYTVTATNSAGTA |
| | | TENLSVIVLEKPGPPVGPVRFDEVSADFVVISWEPPAYTGGCQISNYIVEKRDTTTTTVH |
| | | MVSATVARTTIKITKLKTGTEYQFRIFAENRYGKSAPLDSKAVIVQYPFKEPGPPGTPFV |
| | | TSISKDQMLVQWHEPVNDGGTKIIGYHLEQKKCNSILWVKLNKTPIQDTKFKTTGLDEGL |
| | | EYEFKVSAENIVGIGKPSKVSECFVARDPCDPPGRPEAIVITRNNVTLKWKKPAYDGGSK |
| | | ITGYIVEKKDLPDGRWMKASFTNVLETEFTVSGLVEDQRYEFRVIARNAAGNFSEPSDSS |
| | | GAITARDEIDAPNASLDPKYKDVIVVHAGETFVLEADIRGKPIPDVVWSKDGKELEETAA |
| | | RMEIKSTIQKTTLVVKDCIRTDGGQYILKLSNVGGTKSIPITVKVLDRPGPPEGPLKVTG |
| | | VTAEKCYLAWNPPLQDGGANISHYIIEKRETSRLSWTQVSTEVQALNYKVTKLLPGNEYI |
| | | FRVMAVNKYGIGEPLESGPVTACNPYKPPGPPSTPEVSAITKDSMVVTWARPVDDGGTEI |
| | | EGYILEKRDKEGVRWTKCNKKTLTDLRLRVTGLTEGHSYEFRVAAENAAGVGEPSEPSVF |
| | | YRACDALYPPGPPSNPKVTDTSRSSVSLAWSKPIYDGGAPVKGYVVEVKEAAADEWTTCT |
| | | PPTGLQGKQFTVTKLKENTEYNFRICAINSEGVGEPATLPGSVVAQERIEPPEIELDADL |
| | | RKVVVLRASATLRLFVTIKGRPEPEVKWEKAEGILTDRAQIEVTSSFTMLVIDNVTRFDS |
| | | GRYNLTLENNSGSKTAFVNVRVLDSPSAPVNLTIREVKKDSVTLSWEPPLIDGGAKITNY |
| | | IVEKRETTRKAYATITNNCTKTTFRIENLQEGCSYYFRVLASNEYGIGLPAETTEPVKVS |
| | | EPPLPPGRVTLVDVTRNTATIKWEKPESDGGSKITGYVVEMQTKGSEKWSTCTQVKTLEA |
| | | TISGLTAGEEYVFRVAAVNEKGRSDPRQLGVPVIARDIEIKPSVELPFHTFNVKAREQLK |
| | | IDVPFKGRPQATVNVVRKDGQTLKETTRWVSSSKTVTSLSIKEASKEDVGTYELCVSNSA |
| | | GSITVPITIIVLDRPGPPGPIRIDEVSCDSITISWNPPEYDGGCQISNYIVEKKETTSTT |
| | | WHIVSQAVARTSIKIVRLTTGSEYQFRVCAENRYGKSSYSESSAVVAEYPFSPPGPPGTP |
| | | KVVHATKSTMLVTWQVPVNDGGSRVIGYHLEYKORSSILWSKANKILIADTQMKVSGLDE |
| | | GLMYEYRVYAENIAGIGKCSKSCEPVPARDPCDPPGQPEVTNITRKSVSLKWSKPHYDGG |
| | | AKITGYIVERRELPDGRWLKCNYTNIQETYFEVTELTEDQRYEFRVFARNAADSVSEPSE |
| | | STGPHVKDOVEPPRVMMDVKFRDVIVVKAGEVLKINADIAGRPLPVISVVAKDGIEIEER |
| | | ARTEIISTDNHTLLTVKDCIRRDTGQYVLTLKNVAGTRSVAVNCKVLDKPGPPAGPLEIN |
| | | GLTAEKCSLSWGRPQEDGGADIDYYIVEKRETSHLAWTICEGELQMTSCKVTKLLKGNEY |
| | | IFRVTGVNKYGVGEPLESVAIKALDPFTVPSPPTSLEITSVTKESMTLCWSRPESDGGSE |
| | | ISGYIIERREKNSLRWVRVNKKPVYDLRVKSTGLREGCEYEYRVYAENAAGLSLPSETSP |
| | | LIRAEDPVFLPSPPSKPKIVDSGKTTITIAWVKPLFDGGAPITGYTVEYKKSDDTDWKTS |
| | | IQSLRGTEYTISGLTTGAEYVFRVKSVNKVGASDPSDSSDPQIAKEREEEPLFDIDSEMR |
| | | KTLIVKAGASFTMTVPFRGRPVPNVLWSKPDTDLRTRAYVDTTDSRTSLTIENANRNDSG |
| | | KYTLTIQNVLSAASLTLWKVLDTPGPPTNITVQDVTKESAVLSVVDVPENOGGAPVKNYH |
| | | IEKREASKKAWVSVTONCNRLSYKVTNLQEGAIYYFRVSGENEFGVGIPAETKEGVKITE |
| | | KPSPPEKLGVTSISKDSVSLTWLKPEHDGGSRIVHYVVEALEKGQKNWVKCAVAKSTHHV |
| | | VSGLRENSEYFFRVFAENQAGLSDPRELLLPVLIKEQLEPPEIDMKNFPSHTVYVRAGSN |
| | | LKVDIPISGKPLPKVTLSRDGVPLXATMRFNTEITAENLTINLKESVTADAGRYEITAAN |
| | | SSGTTKAFINIVVLDRPGPPTGPWISDITEESVTLKVVEPPKYDGGSQVTNYILLKRETS |
| | | TAVWTEVSATVARTMMKVMKLTTGEGYQFRIKAENRFGISDHIDSACVTVKLPYTTPGPP |
| | | STPWVTNVTRESITVGWHEPVSNGGSAVVGYHLEMKDRNSILWQKANKLVIRTTHFKVTT |
| | | ISAGLIYEFRVYAENAAGVGKPSHPSEPVLAIDACEPPRNVRITUISKNSVSLSWQQPAF |
| | | DGGSKITGYIVERRDLPDGRVVTKASFTNVTETQFKSGLTQNSQYEFRVFARNAVGSISN |
| | | PSEWGPITCIDSYGGPVIDLPLEYTEVVKYRAGTSVKLRAGISGKPAPTIEVVYKDDKEL |
| | | QTNALVCVENTTDLASILIKDADRLNSGCYELKLRNAMGSASATIRVQILDKPGPPGGPI |
| | | EFKTVTAEKITLLWRPPADDGGAKITHYIVEKRETSRVVWSMVSEKLEECIITTTKIIKG |
| | | NEYIFRVRAVNKYGIGEPLESDSVVAKNAFVTPGPPGIPEVTKITKNSMTVVWSRPIADG |
| | | GSDISGYFLEKRDKKSLGWFKVLKCTIRDTRQKVTGLTENSDYQYRVCAVNAAGQGPFSE |
| | | PSEFYKAADPIDPPGPPAKIRIADSTKSSITLGWSKPVYDGGSAVTGYVVEIRQGEEEEW |
| | | TTVSTKGEVRTTEYVVSNLKPGVNYYFRVSAVNCAGQGEPIEMNEPVQAKDILEAPEIDL |
| | | DVALRTSVIAKAGEDVQVUPFKGRPPPTVTXVHKDEKNLGSDARYSIENTDSSSLLTIPQ |
| | | VTRNDTGKYILTIENGVGEPKSSTVSVKVLDTPAACQKLQVKHVSRGIVTLLWDPPLIDG |
| | | GSPIINYVIEKRDATKRTVVSVVSHKCSSTSFKLIDLSEKTFFFRVLAENEIGIGEPCET |
| | | TQPVKAAEVPAPIRDLSMKDSTKTSVILSWTKPDFDGGSVITEYVVERKGKGEQTWSHAG |
| | | ISKTCEIEVSQLKEQSVLEFRVFAKNEKGLSDPVTIGPITVKELIITPEVDLSDIPGAQV |
| | | TVRIGHNVHLELPYKGKPKPSISWLKDGLPLKESEFVRFSKTENKITLSIKNAKKEHGGK |
| | | YTVILDNAVCRIAVPITVITLGPPSKPKGPIRFDEIKADSVILSWDVPEDNGGGEITCYS |
| | | IEKRETSQTNWKMVCSSVARTTFKVPNLVKDAEYQFRVRAENRYGVSQPLVSSIIVAKHQ |
| | | FRIPGPPGKPVIYNVVSDGMSLTWDAPVYDGGSEVTGFHVEKKERNSILWQKVNTSPISG |
| | | REYRATGLVEGLDYQFRVYAENSAGLSSPSDPSKFTLAVSPVDPPGTPDYIDVIKETITL |
| | | KWNPPLRDGGSKIVGYSIEKRQGNERVWRCNFTDVSECQYTVTGLSPGDRYEFRIIARNA |
| | | VGTISPPSQSSGHMTRDENVPPIVEFGPEYFDGLIIKSGESLRIKALVQGRPVPRVTVVF |
| | | KDGVEIEKRMNMEITDVLGSTSLFVRDATRDHRGVYTVEAKNASGSAKAEIKVKVQDTPG |
| | | KWGPIRFTNITGEKMTLWVVDAPLNDGCAPITHYTEKRETSRLAVVALIEDKCEAQSYTA |
| | | IKLIKGNEYQFRVSAVNKFGVGRPLDSDPVVAQIQYTVPDAPGIPEPSNITGNSITLTWA |
| | | RPESDGGSEIQQYILERREKKSTRWVKVISKRPISETRFKVTGLTEGNEYEFHVMAENAA |
| | | GVGPASGISRLIKCREPVNPPGPPTVVKVTDTSKTTVSLEWSKPVFDGGMEIIGYIIEMC |
| | | KADLGDWHKVNAEACVKTRYTVTDLQAGEEYKFRVSAINGAGKGDSCEVTGTIKAVDRLT |
| | | APELDIDANFKQTHVVRAGASIRLFIAYQORPTPTAVWSKPDSNLSLRADIHTTDSFSTL |
| | | TVENCNRNDAGKYTLTVENNSGSKSITFTVKVLDTPGPPGPITFKDVTRGSATLMWDAPL |
| | | LDGGARIHHYVVEKREASRRSWQVISEKCTRQIFKVNDLAEGVPYYFRVSAVNEYGVGEP |
| | | YEMPEPIVATEQPAPPRRLDVVDTSKSSAVLAWLKPDHDGGSRITGYLLEMRQKGSDFWV |
| | | EAGHTKQLTFTVERLVEKTEYEFRVKAKNDAGYSEPREAFSSVIIKEPQIEPTADLTGIT |
| | | NQLITCKAGSPFTIDVPISGRPAPKVTVVKLEEMRLKETDRVSnTTKDRTTLTVKDSMRG |
| | | DSGRYFLTLENTAGVKTFSVTVVVIGRPGPVTGPIEVSSVSAESCVLSWGEPKDGGGTEI |
| | | TNYIVEKRESGTTAWQLVNSSVKRTQIKVTHLTKYMEYSFRVSSENRFGVSKPLESAPII |
| | | AEHPFVPPSAPTRPEVYHVSANAMSIRWEEPYHDGGSKIIGYWVEKKERNTILWVKENKV |
| | | PCLECNYKVTGLVEGLEYQFRTYALNAAGVSKASEASRPIMAQNPVDAPGRPEVTDVTRS |
| | | TVSLIWSAPAYDGGSKVVGYIIERKPVSEVGDGRWLKCNYTIVSDNFFTVTALSEGDTYE |
| | | FRVLAKNAAGVISKGSESTGPVTCRDEYAPPKAELDARLHGDLVTIRAGSDLVLDAAVGG |
| | | KPEPKIITKGDKELDLCEKVSLQYTGKRATAVIKFCORSDSGKYTLTVKKASGTICAVSV |
| | | MVKVLDSPGPCGKLTVSRVTQEKCTLAWSLPQEDGGAEITHYIVERRETSRLNWVIVEGE |
| | | CPTLSYVVTRLIKNNEYIFRVRAVNKYGPGVPVESEPIVARNSFTIPSPPGIPEEVGTGK |
| | | EHIIIQWTKPESDGGNEISNYLVDKREKKSLRWTRVNKDYVVYDTRLKVTSLMEGCDYQF |
| | | RVTAVNAAGNSEPSEASNFISCREPSYTTOPPSAPRWDTTKHSISLAVVTKPMYDGGTDI |
| | | VGYVLEMQEKDTDQWYRVHTNATIRNTEFTVPDLKMGQKYSFRVAAVNVKGMSEYSESIA |
| | | EIEPVERIEFPDLELADDLKKTVTIRAGASLRLMVSVSGRPPPVITWSKQGIDLASRAII |
| | | DTTESYSLLIVDKVNRYDAGKYTIEAENQSGKKSATVLVKVYDTPGPGPSVKVKEVSRDS |
| | | VTITWEIPTIDGGAPVVVYIVEKREAAMRAFKTVTTKCSKTLYRISGLVEGTMYYFRVLP |
| | | ENIYGIGEPCETSDAVLVSEVPLVPAKLEVVDVAKSTVTLAWEKPLYDGGSRLTGYVLEA |
| | | CKAGTERWMKVVTLKPTVLEHTVTSLNEGEQYLFRIRAQNEKGVSEPRETVTAVTVQDLR |
| | | VLPTIDLSTMPQKTIHVPAGRPVELVIPIAGRPPPAASWFFAGSKLRESERVTVETHTKV |
| | | AKLTIRETTIRDTGEYTLELKNVTGTTSETIKVLILDKPGPPTGPIKIDEIDATSITISW |
| | | EPPELDGGAPLSGYWEQRDAHRPGVVLPVSESVTRSTFKFTRLTEGNEYVFRVAATNRFG |
| | | IGSYLQSEVIECRSSIRIPGPPETLQIFDVSRDGMTLTWYPPEDDGGSQVTGYIVERKEV |
| | | RADRWVRVNKVPVTMTRYRSTGLTEGLEYEHRVTAINARGSGKPSRPSKPIVAMDPIAPP |
| | | GKPQNPRVTDTTRTSVSLAWSVPEDEGGSKVTGYLIEMQKVITHEWTKCNTTPTKIREYT |
| | | LTHLPQGAEYRFRVLACNAGGPGEPAEVPGTVKVTEMLEYPDYELDERYQEGIFVRQGGV |
| | | IRLTIPIKGKPFPICKWTKEGQDISKRAMIATSETHTELVIKEADRGDSGTYDLVLENKC |
| | | GKKAVYIKVRVIGSPNSPEGPLEYDDIQVRSVRVSWRPPADDGGADILGYILERREVPKA |
| | | AWYTIDSRVRGTSLVVKGLKENVEYHFRVSAENQFGISKPLKSEEPVTPKTPLNPPEPPS |
| | | NPPEVLDVTKSSVSLSWSRPKDDGGSRVTGYYIERKETSTDKWVRHNKTQITTTMYTVTG |
| | | LVPDAEYQFRIIAQNDVGLSETSPASEPWCKDPFDKPSQPGELEILSISKDSVTLQVVEK |
| | | PECDGGKEILGYWVEYRQSGDSAWKKSNKERIKDKQFTIGGLLEATEYEFRVFAENETGL |
| | | SRPRRTAMSIKTKLTSGEAPGIRKEMKDVTTKLGnAAQLSCQIVGRPLPDIKWYRFGKEL |
| | | IQSRKYKMSSDGRTHTLTVMTEEQEDEGVYTCIATNEVGEVETSSKLLLQATPQFHPGYP |
| | | LKEKYYGAVGSTLRLHVMYIGRPVPAMTVVFHGQKLLQNSEMTIENTEHYTHLVMKNVQR |
| | | KTHAGKYKVQLSNVFGTVDAILDVEIQDKPDKPTGPPVIEALLKNSAVISWKPPADDGGS |
| | | WITNYVVEKCEAKEGAEWQLVSSAISVTTCRIVNITENAGYYFRVSAQNTFGISDPLEVS |
| | | SVVIIKSPFEKPGAPGKPTITAVTKDSCVVAWKPPASDGGAKIRNYYLEKREKKQNKWIS |
| | | VTTEEIRETVFSVKNLIEGLEYEFRVKCENLGGESEWSEISEPITPKSDVPIQAPHFKEE |
| | | LRNLNVRYQSNATLVCKVTGHPKPIVKWYRQGKEIIADGLKYRIQEFKGGYHQLIIASVT |
| | | DDDATVYQVRATNQGGSVSGTASLEVEVPAKIIILPKTLEGMGAVHALRGEWSIKIPFSG |
| | | KPDPVITWQKGQDLIDNNGHYQVIVTRSFTSLVFPNGVERKDAGFYVVCAKNRFGIDQKT |
| | | VELDVADVPDPPRGVKVSDVSRDSVNLTWTEPASDGGSKITNYIVEKCATTAERWLRVGQ |
| | | ARETRYTVINIFGKTSYQFRVIAENKFGLSKPSEPSEPTITKEDKTRAMNYDEEVDETRE |
| | | VSMTKASHSSTKELYEKYMIAEDLGRGEFGIVHRCVETSSKKTYMAKFVKVKGTDQVLVK |
| | | KEISILNIARHRNILHLHESFESMEELVMIFEFISGLDIFERINTSAFELNEREIVSYVH |
| | | QVCEALQFLHSHNIGHFDIRPENIIYQTRRSSTIKIIEFGQARQLKPGDNFRLLFTAPEY |
| | | YAPEVHQHDVVSTATDMWSLGTLVYVLLSGINPFLAETNQQIIENIMNAEYTFDEEAFKE |
| | | ISIEAMDFVDRLLVKERKSRMTASEALQHPWLKQKIERVSIXVIRTLKHRRYYHTLIKKD |
| | | LNMVVSAARISCGGAIRSQKGVSVAKVKVASIEIGPVSGQIMHAVGEEGGHVKYVCKIEN |
| | | YDQSTQVTWYFGVRQLENSEKYEITYEDGVAILYVKDITKLDDGTYRCKVVNDYGEDSSY |
| | | AELFVKGVREVYDYYCRRTMKKIKRRTDTMRLLERPPEFTLPLYNKTAYVGENVRFGVTI |
| | | TVHPEPHVTWYKSGQKIKPGDKDKKYTFESDKGLYQLTINSVTTDDDAEYTVVARNKYGE |
| | | DSCKAKLTVTLHPPPTDSTLRPMFKRLLANAECQEGQSVCFEIRVSGIPPPTLKWEKDGQ |
| | | PLSLGPNIEIIHEGLDYYALHIRDTLPEDTGYYRVTATNTAGSTSCQAHLQVERLRYKKQ |
| | | EFKSKEEHERHVQKQIDKTLRMAEILSGTESVPLTQVAKEALREAAVLYKPAVSTKTVKG |
| | | EFRLEIEEKKEERKLRMPYDVPEPRKYKQTTIEEDQRIKQFVPMSDMKWYKKIRDQYEMP |
| | | GKLDRWQKRPKRIRLSRVVEQFYVMPLPRITDQYRPKWRIPKLSQDDLEIVRPARRRTPS |
| | | PDYDFYYRPRRRSLGDISDEELLLPIDDYLAMKRTEEERLRLEEELELGFSASPPSRSPP |
| | | HFELSSLRYSSPQAHVKVEETRKDFRYSTYHIPTKAEASTSYAELRERHAQAAYRQPKQR |
| | | QRIMAEREDEELLRPVTTTQHLSEYKSELDFMSKEEKSRKKSRRQREVTEITEIEEEYEI |
| | | SKHAQRESSSSASRLLRRRRSLSPTYIELMRPVSELIRSRPQPAEEYEDDTERRSPTPER |
| | | TRPRSPSPVSSERSLSRFERSARFDIFSRYESMKAALKTQKTSERKYEVLSQQPFTLDHA |
| | | PRITLRMRSHRVPCGQHHRFILNVQSKPTAEVKWYHNGVELOESSKIHYTNTSGVLTLEI |
| | | LDCHTDDSGTYRAVCTNYKGEASDYATLDVTGGDYTTYASQRRDEEVPRSVFPELTRTEA |
| | | YAVSSFKKTSEMEASSSVREVKSQMTETRESLSSYEHSASAEMKSAALEEKSLEEKSTTR |
| | | KIKTTLAARILTKPRSMTVYEGESARFSCDTDGEPVPTVTWLRKGQVLSTSARHQVTTTK |
| | | YKSTFEISSVQASDEGNYSYVVENSEGKQEAEFTLTIQKARVTEKAVTPPPRVKSPEPRV |
| | | KSPEWVKSPKRVKSPEPSHPKAVSPTETKPTPTEKVQHLPVSAPPKITQFLKAEASKEIA |
| | | KLTCWESSVLRAKEVTVVYKDGKKLKENGHFQFHYSADGTYELKINNLTESDQGEYVCEI |
| | | SGEGGTSKTNLQFMGQAFKSIHEKVSKISETKKSDQKTTESTVTRKTEPKAPEPISSKPV |
| | | IVTGLQDTTVSSDSVAKFAVKATGEPRPTAIWTKDCKAITQGGKYKLSEDKGGFFLEIHK |
| | | TDI+DSGLYTCTVKNSAGSVSSSCKLTIKAIKDTEAQKVSTQKTSEITPQKKAVVQEEIS |
| | | QKALRSEEIKMSEAKSQEKLALKEEASKVLISEEVKKSAATSLEKSIVHEEITKTSQASE |
| | | EVRTHAEIKAFSTQMSINEGQRLVLKANIAGATDVKWVLKGVELTNSEEYRYGVSGSDQT |
| | | LTIKQASHRDEGILTCISKTKEGIVKCQYDLTLSKELSDAPAFISQPRSQNINEGQNVLF |
| | | TCEISGEPSPEIEWFKNNLPISISSNVSISRSRNVYSLEIRNASVSDSGKYTIKAKNFRG |
| | | QCSATASLMVLPLVEEPSREVVLRTSGDTSLQGSFSSQSVQMSASKQEASFSSFSSSSAS |
| | | SMTEMKFASMSAQSMSSMQESFVEMSSSSFMGISNMTQLESSTSKMLKAGIRGIPPKIEA |
| | | LPSDISIDEGKVLTVACAFTGEPTPEVTWSCGGRKIHSQEQGRFHIENTDDLTTLIIMDV |
| | | QKQDGGLYTLSLGNEFCSDSATVNIHIRSI |
| 41 | Serine-protein kinase ATM | MSLVLNDLLICCRQLEHDRATERKKEVEKFKRLIRDPETIKHLDRHSDSKQGKYLNWDAV |
| | (ATM) | FRFLQKYIQKETRCLRIAKPNVSASTQASRQKKMQEISSLVKYFIKCANRRAPRLKCQOL |
| | | LNYIMDTVKDSSNGAIYGADCSNILLKDILSVRKYWCEISQQQWLELFSVYFRLYLKPSQ |
| | | DVHRVLVARIIHAVTKGCCSQTDGLNSKFLDFFSKAIQCARQEKSSSGLNHILAALTIFL |
| | | KTLAVNFRIRVCELGDEILPTLLYIWTQHRLNDSLKEVIIELFQLQIYIHHPKGAKTQEK |
| | | GAYESTKWRSILYNLYDLLVNEISHIGSRGKYSSGFRNIAVKENLIELMADICHQVFNED |
| | | TRSLEISQSYTTTQRESSDYSVPCKRKKIELGWEVIKDHLQKSQNDFDLVPWLQIATQLI |
| | | SKYPASLPNCELSPLLMILSQLLPQQRHGERTPYVLRCLTEVALCQDKRSNLESSQKSDL |
| | | LKLWNKIWCITFRGISSEQIQAENFGLLGAIIQGSLVEVDREFWKLFTGSACRPSCPAVC |
| | | CLTLALTTSIIVPGTVKMGIEQNMCEVNRSFSLKESIMKWLLFYQLEGDLENSTEVPPLH |
| | | SNFPHLVLEKILVSLTMKNCKAAMNFFQSVPECEHHQKDKEELSFSEVEELFLQTTFDKM |
| | | DFLTIVRECGIEKHQSSIGFSVHQNLXESLDRCLLGLSEQLLNNYSSEITNSETLVRCSR |
| | | LLVGVLGCYCYMGVIAEEEAYKSELFQKAKSLMQCAGESITLFKNKTNEEFRIGSLRNMM |
| | | QLCTRCLSNCTKKSPNKIASGFFLRLLTSKLMNDIADICKSLASFIKKPFDRGEVESMED |
| | | DTNGNLMEVEDQSSMNLFNDYPDSSVSDANEPGESQSTIGAINPLAEEYLSKQDLLFLDM |
| | | LKFLCLCVTTAQTNTVSFRAADIRRKLLMLIDSSTLEPTKSLHLHMYLMLLKELPGEEYP |
| | | LPMEDVLELLKPLSNVCSLYRRDQDVCKTILNHVLHVVKNLGQSNMDSENTRDAQGQFLT |
| | | VIGAFWHLTKERKYIFSVRMALWCLKTLLEADPYSKVVAILNVMGKDFPVNEVFTQFLAD |
| | | NHHQVRMLAAESINRLFQDTKGDSSRLLKALPLKLQQTAFENAYLKAQEGMREMSHSAEN |
| | | PETLDEIYNRKSVLLTLIAVVLSCSPICEKQALFALCKSVKENGLEPHLVKKVLEKVSET |
| | | FGYRRLEDFMASHLDYLVLEWLNLQDTEYNLSSFPFTLLNYTNIEDFYRSCYKVLIPHLV |
| | | IRSMFDEVKSIANQIQEDWKSLLTDCFPKILVNILPYFAYEGTRDSGMAQQRETATKVYD |
| | | MLKSENLLGKQIDHLFISNLPEIVVELLMTLHEPANSSASQSTDLCDFSGDLDPAPNPPH |
| | | FPSHVIKATFAYISNCHKTKLKSILEILSKSPDSYQKILLAICEQAAETNNVYKKHRILK |
| | | IYHLFVSLLLKDIKSGLGDAWAFVLRDVIYTLIHYINQRPSCIMDVSLRSFSLCCDLLSQ |
| | | VCQTAVTYCKDALENHLHVIVGTLIPLVYEQVEVQKQVLDLLKYLVIDNKDNENLYITIK |
| | | LLDPFPDHVVFKDLRITQQKIKYSRGPFSLLEEINHFLSVSVYDALPLTRLEGLKDLRRQ |
| | | LELHKDQMVDIMRASQDNPQDGIMVKLVVNLLQLSKMAINHTGEKEVLEAVGSCLGEVGP |
| | | IDFSTIAIQHSKDASYTKALKLFEDKELQWTFIMLTYLNNTLVEDCVKVRSAAVTCLKNI |
| | | LATKTGHSFWEIYKMTTDPMLAYLQPFRTSRKKFLEVPRFDKENPFEGLDDINLWIPLSE |
| | | NHDIWIKTLTCAFLDSGGTKCEILQLLKPMCEVKTDFCQTVLPYLIHDILLQDTNESWRN |
| | | LLSTHVQGFFTSCLRHFSQTSRSTTPANLDSESEHFFRCCLDKKSQRTMLAVVDYMRRQK |
| | | RPSSGTIFNDAFWLDLNYLEVAKVAQSCAAHFTALLYAEIYADKKSMDDQEKRSLAFEEG |
| | | SQNTTISSLSEKSKEETGISLQDLLLEIYRSIGEPDSLYGCGGGKMLQPITRLRTYEHEA |
| | | MWGKALVTYDLETAIPSSTRQAGIIQALQNLGLCHILSVYLKGLDYENKDWCPELEELHY |
| | | QAAWRNMQWDKCTSVSKEVEGTSYHESLYNALQSLRDREFSTFYESLKYARVKEVEEMCK |
| | | RSLESVYSLYPTLSRLQAIGELESIGELFSRSVTHRQLSEVYIKWQKHSQLLKDSDFSFQ |
| | | EPIMALRTVILEILMEKEMDNSQRECIKDILTKHLVELSILARTFKNTQLPERAIFQIKQ |
| | | YNSVSCGVSEWQLEEAQVFWAKKEQSLALSILKQMIKKLDASCAANNPSLKLTYTECLRV |
| | | CGNWLAETCLENPAVIMQTYLEKAVEVAGNYDGESSDELRNGKMKAFLSLARFSDTQYQR |
| | | IENYMKSSEFENKQALLKRAKEEVGLLREHKIQTNRYTVKVQRELELDELALRALKEDRK |
| | | RFLCKAVENYINCLLSGEEHDMWVFRLCSLWLENSGVSEVNGMMKRDGMKIPTYKFLPLM |
| | | YQLAARMGTKMMGGLGFHEVLNNLISRISMDHPHHTLFIILALANANRDEFLTKPEVARR |
| | | SRITKNVPKQSSQLDEDRTEAANRIICTIRSRRPQMVRSVEALCDAYHLANLDATQVVKT |
| | | QRKGINIPADQPITKLKNLEDVVVPTMEIKVDHTGEYGNLVTIQSFKAEFRLAGGVNLPK |
| | | IIDCVGSDGKERRQLVKGRDDLRQDAVMQQVFQMCNTLLQRNTETRKRKLTICTTKVVPL |
| | | SQRSGVLEWCTGTVPIGEFLVNNEDOAHKRYRPNDFSAFQCQKKMMEVQKKSFEEKYEVF |
| | | MDVCQNFQPVFRYFCMEKFLDPAIWFEKRLAYTRSVATSSIVGYILGLGDRHVQNILINE |
| | | QSAELVHIDLGVAFEQGKILPTPETVPFRLTRDIVDGMGITGVEGVFRRCCEKTMEVMRN |
| | | SQETLLTIVEVLLYDPLFDWTMNPLKALYLQQRPEDETELHPTLNADDQECKRNLSDIDQ |
| | | SFNKVAERVLMRLQEKLKGVEEGTVLSVGGQVNLLIQQAIDPKNLSRLFPGWKAWV |
| 42 | Myeloperoxidase (MPO) | MGVPFFSSLRCMVDLGPCWAGGLTAEMKLIXALAGLLAILATPQPSEGAAPAVLGEVDTS |
| | | LVLSSMEEAKQLVDKAYKERRESIKQRLRSGSASPMELLSYFKQPVAATRTAVRAADYLH |
| | | VALDLLERKLRSLWRRPFNVTDVLTPAQLNVLSKSSGCAYQDVGVTCPEQDKYRTITGMC |
| | | NNRRSPTLGASNRAFVRWLPAEYEDGFSLPYGWTPGVKRNGFPVALARAVSNEIVRFPTD |
| | | QLTPDQERSLMFMQWGQLLDHDLDFTPEPAARASFVTGVNCETSCVQQPPCFPLKIPPND |
| | | PRIKNQADCIPFFRSCPACPGSNITIRNQINALTSFVDASMVYGSEEPLARNLRNMSNQL |
| | | GLLAVNQRFQDNGRALLPFDNLHDDPCLLTNRSARIPCFLAGDTRSSEMPELTSMHTLLL |
| | | REHNRLATELKSLNPRWDGERLYQEARKIVGAMVQIITYRDYLPLVLGPTAMRKYLPTYR |
| | | SYNDSVDPRIANVFTNAFRYGHTLIQPFMFRLDNRYQPMEPNPRVPLSRVFFASWRVVLE |
| | | GGIDPILRGLMATPAKLNRQNQIAVDEIRERLFEQVMRIGLDLPALNMQRSRDHGLPGYN |
| | | AWRRFCGLPQPETVGQLGTVLRNLKLARKLMEQYGTPNNIDIWMGGVSEPLKRKGRVGPL |
| | | LACIIGTQFRKLRDGDRFWWENEGVFSMQQRQALAQISLPRIICDKTGITTVSKNNIFMS |
| | | NSYPRDFVNCSTLPALNLASWREAS |
| 43 | Xanthine | MTADKLVFFVNGRKVVEKNADPETTLLAYLRRKLGLSGTKLGCGEGGCGACTVMLSKYDR |
| | dehydrogenase/oxidase | LQNKIVHFSANACLAPICSLHHVAVTTVEGIGSTKTRLHPVQERIAKSHGSQCGFCTPGI |
| | (XDH) | VMSMYTLLRNQPEPTMEEIENAFQGNLCRCTGYRPILQGFRTFARDGGCrGGDGNNPNCC |
| | | MNQKKDHSVSLSPSLFKPEEFTPLDPTQEPIFPPELLRLKDTPRKQLRFEGERVTWIQAS |
| | | TLKELLDLKAQHPDAKLWGNTEIGIEMKFKNMLFPMIVCPAVVIPELNSVEHGPDGISFG |
| | | AACPLSIVEKTLVDAVAKLPAQKTEVFRGVLEQLRWFAGKQVKSVASVGGNIITASPISD |
| | | LNPVFMASGAKLTLVSRGTRRTVQMDHTFFPGYRKTLLSPEEILLSIEIPYSREGEYFSA |
| | | FKQASRREDDIAKVTSGMRVLFKPGTTEVQELALCYGGMANRTISALKTTORQLSKLWKE |
| | | ELLQDVCAGLAEELHLPPDAPGGMVDFRCTLTLSFFFKFYLTVLQKLGQENLEDKCGKLD |
| | | PTFASATLLFQKDPPADVQLFQEVPKGQSEEDMVGRPLPHLAADMQASGEAVYCDDIPRY |
| | | ENELSLRLVTSTRAHAKIKSIDTSEAKKVPGFVCFISADDVPGSNITGICNDETVFAKDK |
| | | VTCVGHIIGAVVADTPEHTQRAAQGVKITYEELPAIITTEDAIKNNSFYGPELKIEKGDL |
| | | KKGFSEADNVVSGEIYIGGQEHFYLETHCTIAVPKGEAGEMELFVSTQNTMKTQSFVAKM |
| | | LGVPANRIVVRVKRMGGGFGGKETRSTVVSTAVALAAYKTGRPVRCMLDRDEDMLITGGR |
| | | HPFLARYKVGFMKTGTVVALEVDHFSNVGNTQDLSQSIMERALFHMDNCYKIPNIRGTGR |
| | | LCKTNLPSNTAFRGFGGPQGMLIAECWMSEVAVTCGMPAEEVRRKNLYKEGDLTHFNQKL |
| | | EGFTLPRCWEECLASSQYHARKSEVDKFNKENCWKICRGLCHPTKFGISFTVPFLNQAGA |
| | | LLHVYTDGSVLLTHGGTEMGQGLHTKMVQVASRALKIPTSKIYISETSTNTVPNTSPTAA |
| | | SVSADLNGQAVYAACQTILKRLEPYKKKNPSGSWEDWVTAAYMDTVSLSATGFYRTPNLG |
| | | YSFETNSGNPFHYFSYGVACSEVEIDCLTGDHKNLRTDIVMDVGSSLNPAIDIGQVEGAF |
| | | VQGLGLFTLEELHYSPEGSLHTRGPSTYKIPAFGSIPIEFRVSLLRDCPNKKAIYASKAV |
| | | GEPPLFLAASIFFAIKDAIRAARAQHTGNNVKELFRLDSPATPEKIRNACVDKFTTLCVT |
| | | GVPENCKPWSVRV |
| 44 | DNA-dependent protein | MAGSGAGVRCSLLRLQETLSAADRCGAALAGHQLIRGLGQBCVLSSSPAVLALGTSLVFS |
| | kinase catalytic subunit | RDFGLLVFVRKSLNSIEFRECREEILKFLCIFLEKMGQKIAPYSVEIKNTCTSVYTKDRA |
| | (PRKDC) | AKCKIPALDLLIKLLQTFRSSRLMDEFKIGELFSKFYGGLALKKKIPDTVLEKVYELLGL |
| | | LGEVHPSEMINNAENLFRAFLGELKTQMTSAVREPKLPVLAGCLKGLSSLLCNFTKSMEE |
| | | DPQTSREIFNFVLKAIRPQIDLKRYAVPSAGLRLFALHASQFSTCLLDNYVSLFEVLLKW |
| | | CAHTNVELKKAAUSALESFLKQVSNMVAKNAEMHKNKLDYFMEQFYGIIRNVDSNNKELS |
| | | IAIRGYGLFAGPCKVINAKDVDFMYVELIQRCKQMFLTQTDTGDDRVYQMPSFLQSVASV |
| | | LLYLDTVPEVYTPVLEHLVVMQIDSFPQYSPKMQLVCCRAIVKVFLALAAKGPVLRNCIS |
| | | TVVHQGLIRICSKPVVLPKGPESESEDHRASGEVRTGKWKVPTYKDYVDLFRHLLSSDQM |
| | | MDSILAOEAFFSVNSSSESLNHLLYDEFVKSVLKIVEKLDLTLEIQTVGEQENGDEAPGV |
| | | WMIPTSDPAANLHPAKPKDFSAFINLVEFCREILPDKQAEFFEPWVYSFSYELILQSTRL |
| | | PLISGFYKLLSITVRNAKKIKYFEGVSPKSLKHSPEDPEKYSCFALFVKFGKEVAVKMKQ |
| | | YKDELLASCLTFLLSLPHNIIELDVRAYVPALQMAFKLGLSYTPLAEVGLNALEEWSMEE |
| | | DRHVMQPYYKDILCLDGYLKTSALSDETKNNWEVSALSRAAQKGFNIKVVLKHLKKTKNL |
| | | SSNEAISLEEWIRVVQMLGSLGGQINKNLLTVTSSDEMMKSYVAVVDREKRLSFAVPFKE |
| | | MKPVIFLDVFLPRVTELALTASDRQTKVAACELLHSMVMFMLGKATQMPEGGQGAPPMYQ |
| | | LYKRTFPVLLRLACDVDQVTRQLYEPLVMQLIHWFTNNKKFESQDTVALLEAILDGIVDP |
| | | VDSURDFCGRCIREFLKVVSIKQTTPQQQEKSPVNTKSLFKRLYSLALHPNAFKRLGASL |
| | | AFNNIYREFREEESLVEQFVFEALVIYMESLALAIIADEKSLGTIQGCCDAIDHLCIIEK |
| | | KHVSLNKAKKRRLPRGFPPSASLCLLDLVKWLLAHCGRPQTECRHKSIELFYKFVPLLPG |
| | | NRSPNLWLKDVLKEEGVSFLINTFEGGGCGQPSGILAQPTLLYLRGPFSLQATLCWLDLL |
| | | LAALECYNTFIGERIVGALQVLGTEAQSSLLKAVAFFLESIAMHDLIAAEKCFGTGAAGN |
| | | RTSPQEGERYNYSKCTVVVRIMEFTTTLLNTSPEGWKLLKKDLCNTHLMRVLVQTLCEPA |
| | | SIGFNIGDVQVMAHLPDVCVNLMKALKMSPYKDILETHLREKITAQSIEELCAVNLYGPD |
| | | AQVTRSRLAAVVSACKQLHRAGLLHNILPSQSTDLHHSVGTELLSLVYKGTAPGDERQCL |
| | | PSLDLSCKQLASGIXELAFAFGGLCERLVSLLLNPAVLSTASLGSSQGSVIHFSHGEYFY |
| | | SLFSETINTELLKNLDLAVLELMQSSVDNTKMVSAVLNGMLDQSFRERANQKHQGLKLAT |
| | | TILQHWKKCDSWWAKDSPLETKMAVLALLAKILQIDSSVSFNTSHGSFPEVFTTYISLLA |
| | | DTKLOLHLKGQAVTLLPFFTSLTGGSLEEIRRVLEQLIVAHFPMQSREFPPGTPRFNNYV |
| | | DCMKTFLDALELSQSPMLLELMTEVLCREQQHVMEELFQSSFRRIARRGSCVTQVGLLES |
| | | VYEMFRKDDPRLSFTRQSFVDRSLLTLLWHCSLDALREFFSTIVVDAIDVLKSRFTKLNE |
| | | STFDTQITKKMGYYKILDVMYSRLPKDDVHAKESKINQVFHGSCITEGNELTKTLIKLCY |
| | | DAFTENMAGENQLLERRRLYIICAAYNCAISVKCVFNELKFYQGFLFSEKPEKNLLIFEN |
| | | LIDLKRRYNFPVEVEVPMERKKKYIEIRKEAREAANGDSDGPSYMSSLSYLADSTLSEEM |
| | | SQFDFSTGVQSYSYSSQDPRPATGRTRRREQRDPTVHDDVLELEMDELNRHECMAPLTAL |
| | | VKHMHRSLGPPQGEEDSVPRDLPSWMKFLHGKLGNPIVPLNIRLFLAKLVINTEEVFRPY |
| | | AKHWLSPLLQLAASENNGGEGIHYMVVEIVATILSWTGLATPTGVPKDEVLANRLLNFLM |
| | | KHVFHPKRAVFRHNLEIIKTLVECWKDCLSIPYRLIFEKFSGKDPNSKDNSVGIQLLGIV |
| | | MANDLPPYDPQCGIQSSEYFQALVNNMSFVRYKEVYAAAAEVLGLILRYVMERKNILEES |
| | | LCELVAKQLKQHQNTMEDKFIVCLNKVTKSFPPLADRFMNAVFFLLPKFHGVLKTLCLEV |
| | | VLCRVEGMTELYFQLKSKDFVQVMRHRDDERQKVCLDIIYKMMPKLKPVELRELLNPVVE |
| | | FVSHPSTTCREQMYNILMWIHDNYRDPESETDNDSQEIFKLAKDVLIQGLIDENPGLQLI |
| | | IRNFWSHETRLPSNTLDRLLALNSLYSPKIEVHFLSLATNFLLEMTSMSPDYPNPMFEHP |
| | | LSECEFQEYTIDSDWRFRSTVLTPMFVETQASQGTLQTRTQEGSLSARWPVAGQIRATQQ |
| | | QHDFTLTQTADGRSSFDWLTGSSTDPLVDHTSPSSDSLLFAHKRSERLQRAPLKSVGPDF |
| | | GKKRLGLPGDEVDNKVKGAAGRTDLLRLRRRFMRDQEKLSLMYARKGVAEQKREKEIKSE |
| | | LKMKQDAQVVLYRSYRIIGDLPDIQIKHSSLITPLQAVAQRDPHAKQLFSSLFSGILKEM |
| | | DKFKTLSEKNNITQKLLQDFNRFLNTTFSFFPPFVSCIQDISCQHAALLSLDPAAVSAGC |
| | | LASLQQPVGIRLLEEALLRLLPAELPAKRVRGKARLPPDVLRWVELAKLYRSIGEYDVLR |
| | | GIFTSEIGTKQITQSALLAEARSDYSEAAKQYDEALNKQDWVDGEPTEAEKDFWELASLD |
| | | CYNHLAEWKSLEYCSTASIDSENPPDLNKIWSEPFYQETYLPYMIRSKLKLLLQGEADQS |
| | | LLTFIDKAMHGELQKAILELHYSQELSLLYIXQDDVDRAKYYIQNGIQSFMONYSSIDVL |
| | | LHQSRLTKLQSVQALTGIQEFISHSKQGNLSSQVPLKRLLNTVVTNRYPDAKMDPMNIWD |
| | | DIITQRCFFLSKIEEKLTPLPEDNSMNVDQDGDPSDRMEVQEQEEDISSLIRSCKFSMKM |
| | | KMIDSARKQNNFSLAMKLLKELHKESKTRDDWLVSWVQSYCRLSHCRSRSQGCSEQVLTV |
| | | LKTVSLLDENNVSSYLSKNILAFRDQNILLGTTYRIIANALSSEPACLAEIEEQKARRIL |
| | | ELSGSSSEDSEKVIAGLYQRAFQHLSEAVQAAEEEAQPPSWSCGPAAGVIDAYMTLADFC |
| | | DQQLRKEEENASVIDSAELQAYPALVVEKMLKALKLNSNEARKKFPRLLQIIERYPEETL |
| | | SLMTKEISSVPCWQFISWISHMVALLDKDQAVAVQHSVEEITDNYPQAIVYPFIISSESY |
| | | SFKDTSTGHKNKEFVARIKSKLDQGGVIQDFINALDQLSNPELLFKDWSNDVRAELAKTP |
| | | VNKKNIEKMYERMYAALGDPKArGLGAFRRKFIQTFGKEFDKHFGKGGSKLLRMKLSDFN |
| | | DITNMLLLKMNKDSKPPGNLKTCSPWMSDFKVnFLRNELEIPGQYDGRGKPLPEYHVRIA |
| | | GFDERVTVMASLRRPKRILIRGHDEREHPFLVKGGEDLRQDQRVEQLFQVMNGILAQDSA |
| | | CSQRALQLIITYSVWMTSRLGLIEWLENTVTLKDLLLNTMSQEEKAAYLSUPRAPPCEYK |
| | | DWLTKMSGKHDVGAYMLMYKGANRTETVTSFRKRESKVPADLLKRAFVRMSTSPEAFLAL |
| | | RSHFASSHALICISHWILGIGDRHLNNFMVAMETGGVIGIDFGHAFGSATQFLPVPELMP |
| | | FRLTRQFINLMLPMKETGLMYSIMVHALRAFRSDPGLLTNTMDVFVKEPSFDWKNFEQKM |
| | | LKKGGSWIQEINVAEKNWYPRQKICYAKRKLAGANPAVITCDELLLGHEKAPAFRDYVAV |
| | | ARGSKDHNIRAQEPESGLSEETQVKCLMDQATDPNILGRTWEGWEPWM |
| 45 | Spectrin alpha chain, brain | MDPSGVKVLETAEDIQERRQQVLDRYHRFKELSTLRRQKLEDSYRTQFFQRDAEELNKWI |
| | (SPTAN1) | QEKLQIASDENYKNPTNLQGKLQKHQAFEAEVQANSQAIVKLDETGNLMISEGHFASETI |
| | | RTRLMELHRQWELLLEKMREKGIKLLQAQKLVQYLRECEDVMDWINDKEAIVTSEELGQD |
| | | LEHVEVLQKKFEEFQTDMAAHEERVNEVNQFAAKLIQEQHPEEELIKTKQDEVNAAWQRL |
| | | KGLALQRQGKLFGAAEVQRFNRDVDETISWIKEKEQLMASQDFGRDLASVQALLRKHEGL |
| | | ERDLAALEDKVKALCAEADRLQQSHPLSATQIQVKREELITNWEQIRTLAAERHARLNDS |
| | | YRLQRFLADFRDLTSWVTEMKALINADELASDVAGAEALLDRHQEHKGEIDAHEDSFKSA |
| | | DESGQALLAAGHYASDEVREKLTVLSEERAALLELWELRRQQYEQCMDLQLFYRDTEQVD |
| | | NWMSKQEAFLLNEDLGDSLDSVEALLKKHEDFEKSUSAQEEKIIALDEFATKLIQNNHYA |
| | | MEDVATRRDALLSRRNALHERAMRRRAQLADSFHLQQFFRDSDELKSWVNEKMKTATDEA |
| | | YKDPSNLQGKVQKHQAFEAELSANQSRIDALEKAGQKLIDVNHYAKDEVAARMNEVISLW |
| | | KKLLEATELKGIKLREANQQQQFNRNVEDIELWLYEVEGHLASDDYGKDTTNVQNLQKKH |
| | | ALLEADVAAHQDRIDGITIQARQFQDAGHFDAENIKKKQEALVARYEALKEIMVARKQKL |
| | | ADSLRLQQLFRDVEDEETWIREKEPIAASTNRGKDLIGVQNLLKKHQALQAEIAGHEPRI |
| | | KAVTQKGNAMVEEGHFAAEDVKAKLHELNQKWEALKAKASQRRQDLEDSLQAQQYFADAN |
| | | EAESWMREKEPIVGSTDYGKDCDSAEALLKKHEALMSDLSAYGSSIQALREQAQSCRQQV |
| | | APTDDETGKELVLALYDYQEKSPREVTMKKGDILTLLNSTNKDWWKVEVNDRQGFVPAAY |
| | | VKKLDPAQSASRENLLEEQGSIALRQEQIDNQTRITKEAGSVSLRMKQVEELYHSLLELG |
| | | EKRKGMLEKSCKKFMLFREANELQQWINEKEAALTSEEVGADLEQVEVLQKKFDDFQKDL |
| | | KANESRLKDINKVAEDLESEGLMAEEVQAVQQQEVYGMMPRDETDSKTASPWKSARLMVH |
| | | TVATFNSIKELNERWRSLQQLAEERSQLLGSAHEVQRFHRDADETKEWCEEKNQALNTDN |
| | | YGHDLASVQALQRKHEGFERDLAALGDKVNSLGETAERLIQSHPESAEDLQEKCTELNQA |
| | | WSSLGKRADQRKAKLGDSHDLQRFLSDFRDLMSWINGIRGLVSSDELAKDVTGAEALLER |
| | | HQEHRTEIDARAGTFQAFEQFGQQLLAHGHYASPEIKQKLDILDQERADLEKAWVQRRMM |
| | | LDQCLELQLFHRDCEQAENWMAAREAFLNTEDKGDSLDSVEALIKKHEDFDKAINVQEEK |
| | | IAALQAFADQLIAADHYAKGDISSRRNEVLDRWRRLKAQMIEKRSKLGESQTLQQFSRDV |
| | | DEIEAWISEKLQTASDESYKDPTNIQSKHQKHQAFEAELHANADRIRGVIDMGNSLIERG |
| | | ACAGSEDAVKARLAALADQWQFLVQKSAEKSQKLKEANKQQNFNTGIKDFDFWLSEVEAL |
| | | LASEDYGKDLASVNNLLKKHQLLEADISAHEDRLKDLNSQADSLMTSSAFDTSQVKDKRD |
| | | TINGRFQKIKSMAASRRAKLNESHRLHQFFRDMDDEESWIKEKKLLVGSEDYGRDLTGVQ |
| | | NLRKKHKRLEAELAAHEPAIQGVLDTGKKLSDDNTIGKEEIQQRLAQFVEHWKELKQLAA |
| | | ARGQRLEESLEYQQFVANVEEEEAWINEKMTLVASEDYGDTKVAIQGLLKKHEAFETDFT |
| | | VHKDRVNDVCTNGQDLIKKNNHHEENISSKMKGLNGKVSDLEKAAAQRKAKLDENSAFLQ |
| | | FNWKADVVESWIGEKENSLKTDDYGRDLSSVQTLLTKQETFDAGLQAFQQEGIANITALK |
| | | DQLLAAKHVQSKAIEARHASLMKRWSQLLANSAARKKKLLEAQSHFRKVEDLFLTFAKKA |
| | | SAFNSWFENAEEDLTDPVRCNSLEEIKALREAHDAFRSSLSSAQADFNQLAELDRQIKSF |
| | | RVASNPYTWFTMEALEETWRNLQKIIKERELELQKEQRRQEENDKLRQEFAQHANAFHQW |
| | | IQETRTYLLDGSCMVEESGTLESQLEATKRKHQEIRAMRSQLKKIEDLGAAMEEALILDN |
| | | KYTHESTVGLAQQWDQLDQLGMRMQHNLEQQIQARNTTGVTEEALKEFSMMFKHFDKDKS |
| | | GRLNHQEFKSCLRSLGYDLPMVEEGEPDPEFEAILQTVDPNRDGHVSLQEYMAFMISRET |
| | | ENVKSSEEIESAFRALSSEGKPYVTKEELYQNLTREQADYCVSHMKPYVDGKGRELPTAF |
| | | DYVEFTRSLFVN |
| 46 | Eukaryotic initiation factor | MSGGSADYNREHGGPEGMDPDGVIESNWNEIVDNFDDMNLKESLLRGIYAYGFEKPSAIQ |
| | 4A-II (EIF4A2) | QRAIIPCIKGYDVIAQAQSGTGKTATFAISILQQLEIEFKETQALVLAPTRELAQQIQKV |
| | | ILALGDYMGATCHACIGGTNVRNEMQKLQAEAPHIVVGTPGRVFDMLNRRYLSPKWIKMF |
| | | VLDEADEMLSRGFKDQIYEIFQKLNTSIQVVLLSATMPTDVLEVTKKFMRDPIRILVKKE |
| | | ELTLEGIKQFYINVEREEWKLDTLCDLYETLTITQAVIFLNTRRKVDWLTEKMHARDFTV |
| | | SALHGDMDQKERDVIMREFRSGSSRVLITTDLLARGIDVQQVSLVINYDLPTNRENYIHR |
| | | IGRGGRFGRKGVAINFVTEEDKRILRDIETFYNTTVEEMPMNVADLI |

TABLE 3
Predicted binding affinities of wild-type versus mutant polypeptides of IF4A2

| [symbol] | | | | | [symbol] | | | | |
|---|---|---|---|---|---|---|---|---|---|
| allele | position | length | subseq | IC50 | allele | position | length | subseq | IC50 |
| HLA | 24:3-11 | 9 | FYLDEAUEM | [symbol] | HLA | 24:3-11 | 9 | [symbol] | 17.3 |
| A*0201 | | | | | A*0201 | | | | |
| HLA | 24:3-12 | 10 | FLYDEADEML | 58.9 | HLA | 24:3-12 | 10 | [symbol] | [symbol] |
| A*0201 | | | | | A*0201 | | | | |
| HLA | 24:4-12 | [symbol] | VLDEAUEML | 31.8 | HLA | 24:4-12 | 9 | [symbol] | [symbol] |
| A*0201 | | | | | [symbol] | | | | |
| HLA | 24:3-11 | 9 | FYLEADEM | 131.3 | HLA | 24:3-11 | 9 | [symbol] | [symbol] |
| A*0202 | | | | | A*0202 | | | | |
| HLA | 24:3-12 | 10 | [symbol] | 29.5 | HLA | 24:3-12 | 10 | [symbol] | 7.6 |
| A*0202 | | | | | A*0202 | | | | |
| HLA | 24:3-11 | 9 | [symbol] | 33.2 | HLA | 24:4-12 | 9 | [symbol] | [symbol] |
| A*0202 | | | | | A*0202 | | | | |
| HLA | 24:3-12 | 9 | [symbol] | >500 | HLA | 24:3-11 | 9 | [symbol] | [symbol] |
| A*0203 | | | | | A*0203 | | | | |
| HLA | 24:2-13 | 10 | [symbol] | >500 | HLA | 24:3-12 | 10 | [symbol] | 78.9 |
| A*0203 | | | | | A*0206 | | | | |
| HLA | 24:3-12 | 9 | [symbol] | 18.3 | HLA | 24:3-11 | 9 | [symbol] | [symbol] |
| A*0204 | | | | | A*0206 | | | | |
| HLA | 24:4-12 | 10 | [symbol] | 43.8 | HLA | 24:3-12 | 10 | [symbol] | [symbol] |
| [symbol] | | | | | A*0200 | | | | |
| HLA | 24:3-11 | [symbol] | [symbol] | 117.1 | HLA | 24:4-12 | [symbol] | [symbol] | 77.5 |
| [symbol] | | | | | A*0206 | | | | |
| HLA | 24:4-12 | 5 | [symbol] | 213.4 | HLA | 24:3-11 | [symbol] | [symbol] | 4.7 |
| A*0211 | | | | | [symbol] | | | | |
| HLA | 24:3-11 | 9 | [symbol] | 13.7 | HLA | 24:4-12 | [symbol] | [symbol] | 6 |
| A*0211 | | | | | [symbol] | | | | |
| HLA | 24:4-12 | [symbol] | [symbol] | 463.3 | HLA | 24:3-11 | [symbol] | [symbol] | 6.2 |
| A*0212 | | | | | [symbol] | | | | |
| HLA | 24:3-11 | [symbol] | [symbol] | 19.5 | HLA | 24:4-12 | [symbol] | [symbol] | [symbol] |
| A*0212 | | | | | [symbol] | | | | |
| HLA | 24:4-12 | [symbol] | [symbol] | >500 | HLA | 24:3-11 | [symbol] | [symbol] | 10.7 |
| A*0216 | | | | | [symbol] | | | | |
| HLA | 24:3-11 | [symbol] | [symbol] | 8.5 | HLA | 24:4-12 | [symbol] | [symbol] | 9.5 |
| A*0216 | | | | | [symbol] | | | | |
| HLA | 24:4-12 | [symbol] | [symbol] | 301 | HLA | 24:3-11 | [symbol] | [symbol] | 6.8 |
| A*0219 | | | | | [symbol] | | | | |
| HLA | 24:3-11 | [symbol] | [symbol] | 15.8 | HLA | 24:4-12 | [symbol] | [symbol] | [symbol] |
| A*0219 | | | | | [symbol] | | | | |
| HLA | 24:4-12 | [symbol] | [symbol] | >500 | HLA | 24:3-11 | [symbol] | [symbol] | [symbol] |
| A*0520 | | | | | [symbol] | | | | |
| HLA | 24:3-11 | [symbol] | [symbol] | 12.6 | HLA | 24:4-12 | [symbol] | [symbol] | [symbol] |
| A*0250 | | | | | [symbol] | | | | |
| HLA | 24:3-11 | [symbol] | [symbol] | 248.5 | HLA | 24:2-11 | [symbol] | [symbol] | >500 |
| A*2602 | | | | | [symbol] | | | | |
| HLA | 24:3-11 | [symbol] | [symbol] | >500 | HLA | 24:1-9 | [symbol] | [symbol] | [symbol] |
| A*3201 | | | | | [symbol] | | | | |
| HLA | 24:3-11 | [symbol] | [symbol] | 36.4 | HLA | 24:3-11 | [symbol] | [symbol] | >500 |
| [symbol] | | | | | [symbol] | | | | |
| HLA | 24:3-11 | [symbol] | [symbol] | 56.4 | HLA | 24:3-11 | [symbol] | | [symbol] |
| [symbol] | | | | | [symbol] | | | | |

| | |
|---|---|
| SEQ ID NO: 47 | SEQ ID NO: 48 |
| SEQ ID NO: 49 | SEQ ID NO: 50 |
| SEQ ID NO: 51 | SEQ ID NO: 52 |
| SEQ ID NO: 53 | SEQ ID NO: 54 |
| SEQ ID NO: 49 | SEQ ID NO: [symbol] |
| SEQ ID NO: 51 | SEQ ID NO: 52 |
| SEQ ID NO: 53 | SEQ ID NO: 54 |
| SEQ ID NO: 49 | SEQ ID NO: 50 |
| SEQ ID NO: [symbol] | SEQ ID NO: 52 |
| SEQ ID NO: 49 | SEQ ID NO: 50 |
| SEQ ID NO: [symbol] | SEQ ID NO: 52 |
| SEQ ID NO: 53 | SEQ ID NO: 54 |
| SEQ ID NO: 49 | SEQ ID NO: 50 |
| SEQ ID NO: [symbol] | SEQ ID NO: [symbol] |
| SEQ ID NO: 49 | SEQ ID NO: 50 |
| SEQ ID NO: 53 | SEQ ID NO: 54 |
| SEQ ID NO: 49 | SEQ ID NO: 50 |
| SEQ ID NO: 53 | SEQ ID NO: [symbol] |
| SEQ ID NO: 49 | SEQ ID NO: 50 |
| SEQ ID NO: [symbol] | SEQ ID NO: 54 |
| SEQ ID NO: 40 | SEQ ID NO: 50 |
| SEQ ID NO: 53 | SEQ ID NO: 54 |
| SEQ ID NO: 49 | SEQ ID NO: [symbol] |
| SEQ ID NO: [symbol] | SEQ ID NO: [symbol] |
| SEQ ID NO: 49 | SEQ ID NO: 50 |
| SEQ ID NO: 49 | SEQ ID NO: 50 |

[symbol]

indicates data missing or illegible when filed

What is claimed is:

1. A method for increasing a patient's immune response to tumor cells, comprising administering to a patient with a tumor an amount effective of one or more of the polypeptides comprising the amino acid sequence SEQ ID Nos. 1-23, in an amount effective to increase the patient's immune response to cells of the tumor.

2. The method of claim 2, further comprising treating the patient with traditional cancer immunotherapy, wherein the one or more polypeptides are administered prior to the traditional cancer immunotherapy to enhance efficacy of the traditional cancer immunotherapy.
